SEQUENCE LISTING (1 sequence)

SEQ ID NO: 1 - protein, 386 aa, Artificial Sequence
  Synthetic Polypeptide
MSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYMGFDSQDGKRVNEIEFLTENQKIFVCGNSISVEVLEAIIDKIGG

(19) United States
(12) Patent Application Publication
(10) Pub. No.: US 2017/0369855 A1
(43) Pub. Date: Dec. 28, 2017

(54) SYSTEMS AND METHODS FOR GENOME MODIFICATION AND REGULATION

(71) Applicants: DANA-FARBER CANCER INSTITUTE, INC., Boston, MA (US); The Johns Hopkins University, Baltimore, MD (US)

(72) Inventors: Carl NOVINA, Newton, MA (US); Glenna MEISTER, Boston, MA (US); Marc OSTERMEIER, Baltimore, MD (US); Tina XIONG, Baltimore, MD (US)

(21) Appl. No.: 15/539,256

(22) PCT Filed: Dec. 24, 2015
(86) PCT No.: PCT/IB2015/059984

§ 371 Date: Jun. 23, 2017

Related U.S. Application Data
(60) Provisional application No. 62/096,766, filed on Dec. 24, 2014.
(60) Provisional application No. 62/143,080, filed on Apr. 4, 2015.
(60) Provisional application No. 62/186,862, filed on Jun. 30, 2015.

(51) Int. Cl. C12N 9/10; C12Q 1/68; C12N 15/11; C12N 9/22; C12N 2310/20

(52) U.S. Cl. CPC C12N 9/1007; C12Y 301/00; C12Y 201/01037; C12N 15/11; C12N 9/22; C12Q 1/6897; C07K 2319/09; C07K 2319/81; C12N 2310/20; C07K 2319/80; C12Q 2600/154

(57) ABSTRACT

The present invention provides methods of systems and methods of site specific methylation.

### RELATED APPLICATIONS

This application claims priority to, and the benefit of U.S. Provisional Application No. 62/096,766 filed on Dec. 24, 2015, U.S. Provisional Application No. 62/143,080 filed on Apr. 4, 2015, and U.S. Provisional Application No. 62/186,862 tiled on Jun. 30, 2015 the contents of each of which are incorporated herein by reference in their entirety.

### GOVERNMENT INTEREST

This invention was made with government support under 1DP1 DK105602-01 awarded by the National Institutes of Health. The government has certain rights in the invention.

### FIELD OF THE INVENTION

The present invention relates generally to compositions and methods of gene modification.

### BACKGROUND OF THE INVENTION

The DNA methylation of eukaryotic promoters is a heritable epigenetic modification that causes transcriptional repression. Methylation is implicated in numerous cellular processes such as DNA imprinting and cellular differentiation. Abnormal methylation patterns have also been associated with cancer and diseases caused by deregulation of imprinted genes. In general, hypermethylated promoters are repressed and hypomethylated promoters are not.

There are a variety of mechanisms by which methylation can result in downregulation of gene expression. Methyl CpG-binding domain proteins bind to hypermethylated regions of DNA recruiting histone deacetylases and other corepressors that alter chromatin and inhibit transcription. In addition, methylation within a transcription factor binding site can attenuate transcription by directly preventing the binding of transcription factors or indirectly by recruiting methyl CpG-binding domain proteins that block the transcription factor binding site. There is a growing body of work indicating that downregulation of expression greatly depends on the location of methylation in the promoter. Although there is some evidence that methylation of single CpG sites may downregulate expression, promoters of silenced genes are usually methylated at many sites. Thus a need exists for the ability to site-specifically alter many CpG sites in a promoter.

### SUMMARY OF THE INVENTION

In various aspects the invention provides a system containing a bifurcated enzyme having a first fragment and a second fragment. The first, second or both fragment each further have a DNA binding domain that bind elements flanking a target region. The system has been optimized for expression in mammalian cells. The first fragment comprises the N-terminal portion of the enzyme and the second portion comprises yje C-terminal portion of the enzyme. In preferred embodiments the second fragment comprises the DNA binding domain. The DNA binding domain of the binds elements upstream or downstream of the target region. Optionally there is a linker between the enzyme fragment and the DNA binding domain. In some aspects the system comprises a nuclear localization signal. In some aspects the enzyme is a DNA methyltransferase or DNA demethylase. The target region contains a CpG methylation site. The target region is within a promoter region.

In preferred embodiments, the enzyme is a DNA methyltransferase. The first fragment comprises a portion of the catalytic domain of the DNA methyltransferase. The DNA methyltransferase is M.SssI. The first fragment comprises amino acids 1-272 of the M.SssI. The second fragment comprises amino acids 273-386 of the M.SssI.

The DNA binding domain is for example, a zinc finger, a TAL effector DNA-binding domain or a RNA-guided endonuclease and a guide RNA. The guide RNA is complementary to the region flanking the target region. The RNA-guided endonuclease is for example a CAS9 protein. The CAS9 protein has inactivated nuclease activity.

Also included in the invention is a plurality of systems according to the invention wherein the DNA binding domain of each system binds a different site in genomic DNA.

The invention further includes a fusion protein having an RNA guided nuclease such as a CAS9 protein and a first portion of a bifurcated methyltransferase. The fusion protein is expressed in a mammalian cell.

In another aspect the invention provides an expression cassette having a nucleic acid encoding a bifurcated methyltransferase, a DNA binding domain and a mammalian promoter and mammalian cells expressing the cassette.

In yet a further aspect the invention provide a reporter plasmid having a backbone free of any methylation sites having a target promoter sequence inserted upstream of a nucleic acid encoding a first fluorescent protein and a control promoter sequences inserted upstream of a nucleic acid encoding a second fluorescent protein. The first fluorescent protein is mCherry and the second fluorescent protein is mTAGBFP2. The target promoter is methylation sensitive. The control promoter is not methylation sensitive. For example, the control promoter is CpG free EF1. Alternatively, both the target promoter and the control promoter is methylation sensitive. Cells containing the plasmid of the invention are also provided. In some aspects the cell further includes an expression plasmid comprising a DNA demethylase or DNA methyltransferase fused to a DNA binding domain.

In various aspects the invention further provides a method of identifying a functionally repressive CpG site in a target promoter by a cell according to the invention with a plurality of guide RNAs and measuring the fluorescent intensity of the first and second fluorescent protein.

The invention also includes a method of epigenetic reprogramming a cell by contacting the cell with the system according to the invention.

In another aspect the invention provides a method of epigenetic therapy by administering to a subject in need thereof a composition comprising the system according to the invention.

The subject has cancer, a hematologic disorder, a neurodenerative disorder, heart disease, diabetes, or mental illness. The hematologic disorder is for example sickle cell or thalessemia. The cancer is for example lymphoma.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 is a series of schematics that depict strategies for targeted methylation. (A) A natural DNA (methyltransferase) MTase methylates frequently in DNA since the recognition site is short (typically 2-4 bases) (B) End-to-end fusions of a MTase with a DNA-binding domains designed to bind near the target site for methylation¹⁻⁸ shows bias for the target site but suffers from significant off-target methylation since binding of the DNA-binding domain is not required for enzyme activity. (C) Our strategy provides a mechanism for engineering specificity. An artificially split DNA methyltransferase is incapable of assembling into an active enzyme on its own, but binding to the target DNA facilitates templated assembly of an active MTase at the target site.

FIG. 2 is a series of schematics and a gel that depict the restriction enzyme protection assay for targeted methylation. (A) A single plasmid encodes genes for both MTase fragment proteins, as well as two sites for assessing the degree of targeted methyltransferase activity. Expression of both protein fragments is induced and plasmid DNA is isolated from an overnight cell culture. (B) Plasmid DNA is linearized by SacI digestion and incubated with FspI, an endonuclease whose activity is blocked by methylation. (C) Mock electrophoretic gel showing pattern for 1) inactive methyltransferase, 2) enzyme methylating site 1 only, 3) enzyme methylating site 2 only, 4) enzyme methylating both sites.

FIG. 3 is a schematic that depicts the S. pyogenes Cas9-gRNA complex. Target recognition requires protospacer sequence complementary to the spacer and presence of the NGG PAM sequence at the 3′ of the protospacer. Figure adapted from Mali et al.

FIG. 4 is a series of graphs that depict bisulfite analysis of methylation (A) at and near the target site and (B) far away from the target site for ZF-M.SssI MTase on a plasmid in E. coli9. Percent methylation observed at individual CpG sites was determined by bisulfite sequencing of n clones (n indicated at right). CpG sites are numbered sequentially from 1-48 or 1-60 based on their order in the sequencing read and thus, the figure does not indicate the distance between sites. Black, ‘WT’ heterodimeric enzyme (KFNSE); orange, PFCSY variant; blue, CFESY variant. Variants are named for the protein sequence in the site that was mutated. The arrow indicates the target site

FIG. 5 is a schematic and gels that depict biased methylation using split M.SssI fused to dCas9. (A) schematic of the split MTase bound at a target site, (B) Restriction enzyme protection assay showing periodicity on methylation activity based on the spacing between the PAM site and target site for methylation. The split MTase was coexpressed with gRNA targeting site 1. (C) Demonstration of modularity. The same fusion protein is expressed in both halves of the gel, the only difference is whether gRNA targeting site 1 or site 2 is expressed, For the gels of (B) and (C) the bands indicating methylation at the indicated sites are identified (see FIG. 2 for background on the assay). Expression refers to expression of the split MTase. gRNA was constitutively expressed.

FIG. 6 is a general schematic of dCas9-M.SssI split MTase. Orthogonal dCas9s will be used. The PAM sites for S. pyrogenes are shown as an example.

FIG. 7 is a schematic that depicts in vitro selection for targeted MTases9. The schematic illustrates the fates of plasmids encoding inactive MTase (which is digested by FspI, left), a nonspecific MTase methylating multiple M.SssI sites (which is digested by McrBC, right) and a desired targeted MTase which specifically methylates the on-target site (which is digested by neither, middle). The 3- to 5′ exonuclease activity of ExoIII degrades the DNA encoding undesired library member. Although it is not explicitly shown in this figure, this selection strategy can be implemented in a two-plasmid system as long as the mutagenesis and target site for methylation are located on the same plasmid.

FIG. 8 are a series of gels that depict additional evidence of targeted methylation at different gap lengths. Results of a restriction enzyme protection assay are shown for the split MTase S.pyog dCas9-(GGGGS)₃-M.SssI[273-386] and M.SssI [1-272]. (A) Demonstration of how induction levels of both fragments effect targeted methylation. S.pyog dCas9-(GGGGS)₃-M.SssI[273-386] is induced by arabinose while M.SssI [1-272] is induced by IPTG. Induction of both fragments results in the greatest methylation at the target sites (site 1), but also has higher levels of off-target methylation. The result points to the synergistic effect on methylation from the assembly of both fragments. The fact that both promoters are leaky in the absence of inducer can explain the low level of methylation when only the expression of one of the two fragments is induced. (B) Additional evidence of how the gap length's effect on targeted methylation has a periodicity. All lanes used plasmid isolated from cells grown in the presence of both IPTG and arabinose. The sgRNA used in this experiment also targeted site 1 for methylation.

FIG. 9 is a gel that depicts targeted methylation requires the sgRNA. Results of a restriction enzyme protection assay are shown. The split MTase used in this figure is S.pyog dCas9-(GGGGS)₃-M.SssI[273-386] and M.SssI [1-272]. Both parts of the MTase were induced. The only difference between the two lanes is whether the sgRNA1 was present on the plasmid or was absent.

FIG. 10 is a series of schematics that depict modified S.pyog dCas9 and M.SssI fusions for expression in mammalian cells. (A) The S.pyog dCas9-(GGGGS)₃-M.SssI[273-386] and M.SssI [1-272] fragments codon optimized for mammalian cells. In addition nuclear localization signals (NLS) and tags were added the N-termini of both constructs. Modified constructs were then moved into mammalian expression vectors with the S.pyog dCas9-(GGGGS)₃-M.SssI[273-386] and M.SssI [1-272] fragments under control of a CMV promoter with an IRES (internal ribosome entry site) between the dCas9 fusion and M.SssI [1-272] fragment (B) or only the S.pyog dCas9-(GGGGS)₃-M.SssI[273-386] expressed under CMV with the IRES removed (C). Both vectors also contain a sgRNA expressed under a U6 promoter and GFP expressed by the SFFV promoter.

FIG. 11 is a series of schematics and a graph that depict targeted methylation at the HBG1 promoter. (A) Schematic of the testing of the split MTase fragments in HEK293T cells. Plasmids containing either the S.pyog dCas9-(GGGGS)₃-M.SssI[273-386] and M.SssI [1-272] or a plasmid containing only the S.pyog dCas9-(GGGGS)₃-M.SssI[273-386] were transfected into HEK293T cells. Cells were then recovered after 48 hrs and underwent fluorescence activated Cell Sorting (FACS) to isolate GFP positive cells. Genomic DNA from positive cells is then bisulfite converted and sequenced. (B) S.pyog dCas9 is targeted by a sgRNA target sequence (red) upstream of the −53 and −50 CpG sites. Sites are 8 and 11 bp away from the PAM site (blue). (C) Methylated cytosines were determined by bisulfite sequencing and % of sites methylated calculated from cells expressing S.pyog dCas9-(GGGGS)₃-M.SssI[273-386] and M.SssI[1-272] (blue), S.pyog dCas9-(GGGGS)₃-M.SssI[273-386] only (red), and untreated cells containing no vector plasmid (green).

FIG. 12 are a series of schematics and graphs that depict testing of dCas9-M.SssI[273-386] variants with different linkers and NLS configurations. Schematics of the different variants tested (A). Variants are tested by localizing the dCas9 fusions to site upstream of the −53 and −50 CpG sites in the human HBG I promoter using the F2 sgRNA (B). Schematic showing the expression plasmid and experimental design (C). M.SssI fragments are expressed off a single plasmid and transfected into HEK293T cells. Cells are allowed to grow for 48 hours before FACS sorting to isolate GFP positive cells. These cells are then analyzed by bisulfite conversion and pyrosequencing. Schematics of dCas9-M.SssI[273-386] (C) and M.SssI[1-272] (N) fragments for coexpressed samples and negative controls and expected methylation outcomes are also shown (D). Pyrosequencing primers designed and CpG methylation sights analyzed on the HBG1 promoter (E), Targeted −53 and −50 sites are analyzed on both the top and bottom strands while downstream sites +6 and +17 are only analyzed on the top strand. Data for the top and bottom strands were averaged for the target sites while data is reported for only the top strand for +6 and +17 (F).

FIG. 13 is a schematic that depicts cotransfection of M.SssI expression plasmids for evaluating the methylation activity of constructs on genomic DNA.

FIG. 14 is a series of schematics and graphs that depict the evaluation of methylation activity by different M.SssI[1-272] human optimized variants coexpressed with dCas9-Glink-M.SssI[273-386] v1 1×NLS off separate plasmids. dCas9-M.SssI[273-386] plasmids also express the HBG F2 sgRNA targeting the HBG1 promoter −50/−53 sites. This directs the M.SssI C-terminal fusion protein dCas9-M.SssI[273-386] fragment to the promoter allowing for a free N-terminal M.SssI[1-272] to bind and methylate at the target site (A). Plasmids expressing the dCas9-Glink-M.SssI[273-386] v1 1×NLS were cotransfected in separate wells with plasmids containing one of the four variations of the M.SssI[1-272] varying in the tags, codon optimization and placement and number of NLS sequences (B). Results of DNA methylation at 4 CpG sites on the HBG promoters analyzed by pyrosequencing (C). Top and bottom strand % methylation were averaged for the −50 and −53 sites while +6 and +17 sites were only measured on the top strand.

FIG. 15 is a series of schematic and graphs that depict the Evaluation of methylation activity by different M.SssI[1-272] human optimized variants coexpressed with dCas9-Glink-M.SssI[273-386] v1 1×NLS off separate plasmids. dCas9-M.SssI[273-386] plasmids also express the HBG F2 sgRNA targeting the HBG1 promoter −50/−53 sites. This directs the M.SssI C-terminal fusion protein dCas9-M.SssI[273-386] fragment to the promoter allowing for a free N-terminal M.SssI[1-272] to bind and methylate at the target site (A). Plasmids expressing the dCas9-Glink-M.SssI[273-386] v1 2×NLS or dCas9-Glink-M.SssI[273-386] v2 2×NLS were cotransfected in separate wells with plasmids containing one of 3 variations of the M.SssI[1-2721 (B). Results of DNA methylation at the target CpG sites on the HBG promoters analyzed by pyrosequencing (C). Top and bottom strand % methylation were averaged for the −50 and −53 CpG sites.

FIG. 16 is a series of schematics and graphs that depict the Evaluation of methylation activity of dCas9 and M.SssI[273-386] with different fusion sites. Because the N- and C-termini of dSPCas9 are on opposite sides of the protein (with the C-termini closer to the PAM binding site domain and the N-termini on the opposite side of the protein closer to DNA by the 5′ end of the sgRNA), different sgRNA sequences were designed upsteam of the HBG −53 and −50 sites. The F2 sgRNA is on the top strand while the R2 sgRNA is on the bottom (A). Localizing dCas9 fusions to these sites produce different orientations of the M.SssI[273-386] (C) fragment either towards the target sites or away from the target site (B). dCas9 fusion variants were created using dCas9-Glink-M.SssI[273-386] v1 2×NLS, dCas9-Glink-M.SssI[273-386] v1 2×NLS and a different fusion point with M.SssIP-LFL-dCas9 v2 1×NLS. Each was co expressed with v2 M.SssI[1-272] fragments that were not fused to any dna binding domain proteins (C). Results of DNA methylation at the target CpG sites on the HBG promoters analyzed by pyrosequencing (D). Top and bottom strand % methylation were averaged for the −50 and −53 CpG sites.

FIG. 17 is a series of schematics and graphs that depict the methylation of the human SALL2 P2 promoter. The SALL2 P2 promoter contains a total of 27 CpG sites in the 550 base pairs up stream of the SALL2 E1a translation start site. Within this promoter is a large density of CpG sites qualifying as a CpG island between the CpG 4-27 sites (A). Guide strands were designed to target the CpG sites closest to the translation start site marked by the black box. The SALL2 F1 and SALL2 R1 sgRNA sequences (PAM sites also in bold) are highlighted on the promoter sequence(B). CpG methylation sites are also shown in bold. Methylation levels were evaluated by pyrosequencing in a region on the bottom strand only between CpG sites 18-27. Results are shown for the dCas9-neg-LFL-M.SssI[273-386] coexpressed with the HA-M.SssI[1-272] v2 1×NLS targeted to either the SALL2 F1 sgRNA site or the SALL2 R2 site (C) and results from the same experiment with samples coexpressing the M.SssI-P-LFL-dSPCas9 v2 1NLS and. HA-M.SssI[1-272] v2 1×NLS plotted separately for clarity (D). The relative orientation of the dCas9-M.SssI fusion proteins are shown along with the approximate binding site above the graphs. Each CpG site also lists the relative distance from either the sgRNA PAM site (C) or the last bp of the sgRNA target site (D) depending on which M.SssI fusion site is used. We also evaluated several negative controls in this experiment: Mock (optifect only) and HA-M.SssI[1-272] v2 1×NLS only samples are shown in each graph for reference. In the data set shown in (C) there is an additional negative control of dCas9-neg-LFL-M.SssI[273-386] v2 1×NLS SALL2 F 1 sgRNA only and in the data shown in (D) the coexpression of M.SssI[273-386]-P-LFL-dSPCas9 and HA-M.SssI[1-272] v2 1×NLS but with a sgRNA targeted towards a different site on the genome: the HBG F2 site (D).

### DETAILED DESCRIPTION OF THE INVENTION

The invention provides compositions, systems and methods for targeted methylation that allows the identification and exploitation of site specific methylation effects on promoter activity. In particular embodiments, the systems have been optimized for expression in a mammalian cell. By optimized for expression in a mammalian cell is meant for example, that the modifications have been incorporated in the nucleic acid and or amino acid sequence of the enzyme such the at enzyme can be expressed in a mammalian cell. Additional modifications include promoter modifications, modification in the nuclear localization signal; and mammalian post-translational modifications.

Specifically, the invention provides a system for targeting methylation, based upon a fusion of a bifurcated methyltransferase and a DNA binding domain. The methyltransferase is derived for bacteria and has been optimized for expression in a mammalian cell. Alternatively, the methyltransferase is mammalian. The DNA binding domain is for example, a Helix-turn-helix, a Zinc finger, a Leucine zipper, a Winged helix, a Helix-loop-helix, a HMG-box, a Wor3 domain, an Immunoglobulin fold, a B3 domain, a TAL effector DNA-binding domain or a RNA-guided DNA-binding domain.

Specifically, the invention provides a modular system for targeting methylation, based on RNA-guided DNA-binding domains such as Cas9 protein. The Cas9 protein is an endonuclease that is part of the Clustered Regularly Interspaced Short Palindromic Repeats (CRISPRs) system, an RNA-based adaptive immune system for bacteria in which guide RNA (gRNA) are used to target Cas9 nuclease activity to specific sequences in foreign DNA. The modular nature of Cas9 recognition of DNA, as recognition of DNA is programmed by changes to the gRNA using the simple base-pairing rules of DNA. By knocking out the nuclease activity of Cas9 through mutation to create endonuclease deficient Cas9 (dCas9) proteins, Cas9 is converted into a modular DNA binding protein, which can be use to target epigenetic modifying enzymes to DNA dCas9 is the optimal protein to facilitate epigenetic reprogramming by site-specific DNA methylation. A single dCas9-MTase fusion protein can be directed to multiple different sites within a promoter or to multiple different promoters simply by transducing cells with different gRNAs (i.e. new DNA binding modules are not required to recruit a particular enzyme to a unique sequence). Instead, a common dCas9-MTase fusion protein is recruited to multiple different CpGs within a promoter, which vastly improves gene silencing efficiency.

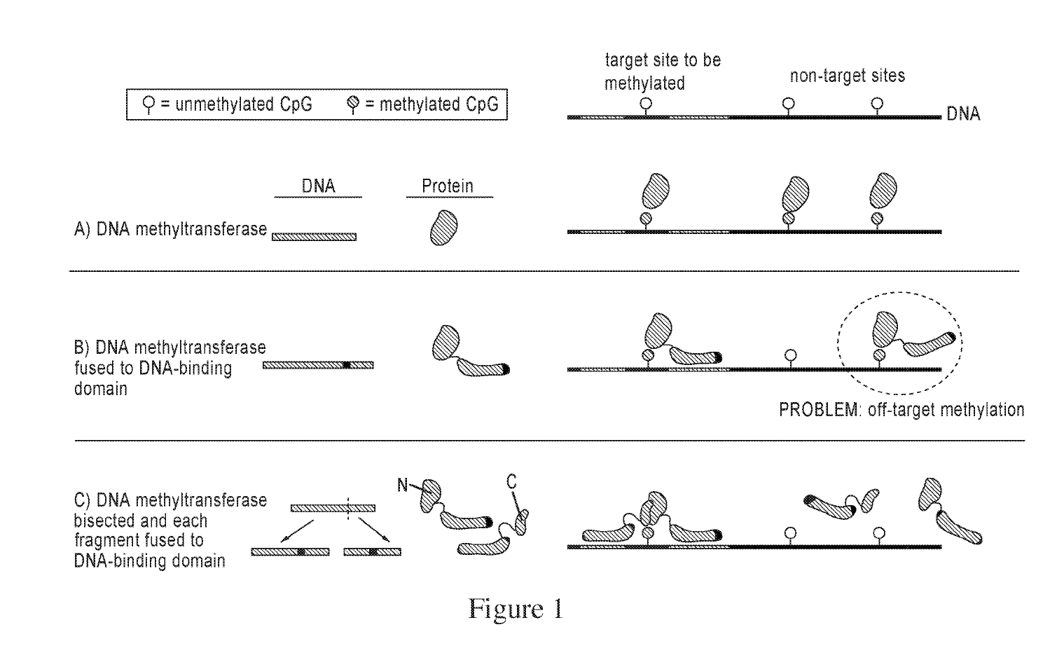

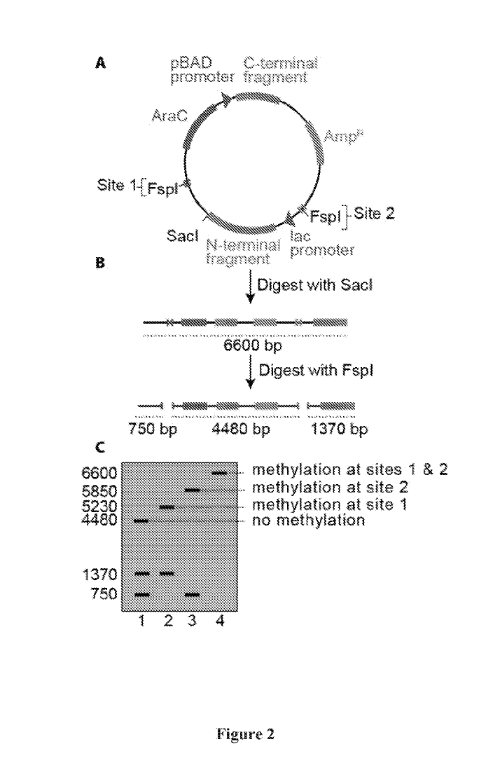

In order to target CpG methylation using dCas9 methyltransferase (MTase) activity must require the association of the fused DNA binding domain with its recognition site. To achieve this, the present invention employs splitting the naturally monomeric MTase into two fragments and fusing one or both of the fragments to different DNA binding domains that bind elements flanking the target CpG site for methylation. (FIG. 1C). Association of the DNA binding domain with its recognition site facilitates the proper assembly of the fragmented MTase only at the desired CpG site. For example, when both fragments are bound to proximal sites on the DNA, their local, effective concentration increases above the K_{d} and an active MTase is formed only at the target site.

The ability to target site-specific DNA methylation in vivo allows testing of previously untestable hypotheses. As a research tool, the relationships between DNA methylation initiation, spreading, inheritance and the generation of higher-order chromatin structures can be established. Additionally, the compositions and systems of the invention can be used in screening approaches for discovery of gene function in a high-throughput manner or in silencing genes of interest in model organisms. As an epigenetic therapeutic agent compositions and systems of the invention can stably represses a disease-causing target genes.

Gene silencing by targeted methylation has three key advantages over approaches such as antisense-RNA, small interfering RNAs (siRNAs), ribozymes and similar strategies. First, methylation recruits other factors to establish local chromatin structures that further repress expression. Second, methylation patterns and chromatin structures are heritable during cell division. Thus, transient expression of an epigenetic modifying enzyme may lead to stable repression phenotypes. Third, transcription factors are global regulators of gene expression and cell fates. In theory, a targeted MTase need only act on the targeted promoter to inhibit entire transcriptional programs.

Current strategies for targeted methylation have a fundamental design flaw. The strategy consists of genetically fusing MTases to DNA binding domains (usually zinc finger domains, although other localizing agents such as triple helix forming oligonucleotides have been used) to localize the MTase to the targeted site (FIG. 1B). Because the MTase domain is active in the absence of the DNA binding to its target site, the MTase is free to methylate off-target sites (FIG. 1B). Accordingly, analyses of the methylation patterns created using these engineered MTases reveal significant methylation at both on-target and off-target sites. These engineered MTases achieve biased methylation but not specific methylation. This off-target activity substantially limits the use of these fusion proteins as research or therapeutic tool. These biased MTases are far from achieving the targeted methylation necessary to realize the promise of targeted MTases as research tools and therapeutics. In addition, these MTase are not modular, as a new protein must be designed for each new target site. Existing approaches lack a strategy to achieve the desired specificity and modularity. The present invention provides a solution to both of these problems.

In addition, most of the previous studies above lack a rigorous, quantitative assessment of the bias the engineered MTases have for their target site. This deficiency prevents a direct comparison and limits the design and optimization of these MTases. Studies on purified engineered MTases assayed under the non-biological conditions of a large molar excess of target site DNA over enzyme do not appropriately address specificity, because they artificially keep the MTases sequestered at the target site (and thus unavailable to methylate off-target sites).

The present disclosure provides RNA-guided DNA-binding fusion proteins. The fusion proteins comprise CRISPR/Cas-like proteins or fragments thereof and an effector domain, e.g., an epigenetic modification domain. Each fusion protein is guided to a specific chromosomal sequence by a specific guiding RNA, wherein the effector domain mediates targeted genome modification or gene regulation. In a specific embodiment, the effector domain is split into a two fragments. The effector domain is spit in such a way that when the two fragment re-associate they form a functional (i.e., active) enzyme. In some aspects one of the two fragments comprises the entire catalytic domain of the effector domain. In other aspects one of the two fragments comprises the majority of the catalytic domain. Each of the two fragments comprises a DNA binding domain (e.g., Cas 9). Alternatively, only one of the fragments comprises a DNA binding domain. For example the N-terminal fragment of the effector domain comprises a DNA binding domain. Alternatively, the C-terminal fragment of the effector domain comprises a DNA binding domain. Preferably, only the C-terminal fragment of the effector domain comprises a DNA binding domain.

One aspect of the present disclosure provides a fusion protein comprising a CRISPR/Cas-like protein or fragment thereof and an effector domain. The CRISPR/Cas-like protein is derived from a clustered regularly interspersed short palindromic repeats (CRISPR)/CRISPR-associated (Cas) system protein. The effector domain is an epigenetic modification domain. More specifically, the effector domain is a bifurcated epigenetic modification domain. For example, the bifurcated epigenetic domain is a split methyltransferase. Preferably, the methyltransferase is spit such that one portion contains the catalytic domain. In preferred embodiments the methyltransferase is M.SssI. In some embodiments the first fragment comprises amino acids 1-272 of the M.SssI and the second fragment comprises amino acids 273-386 of the M.SssI.

An exemplary M.SssI. amino acid sequence useful in the compositions and methods of the invention shown is SEQ ID NO:1.

| (SEQ ID NO: 1) | | |
|---|---|---|
| 1 | MSKVENKTKKLRVFEAFAGI | 20 |
| 21 | GAQRKALEKVRKDEYEIVGL | 40 |
| 41 | AEWYVPAIVMYQAIHNNFHT | 60 |
| 61 | KLEYKSVSREEMIDYLENKT | 80 |
| 81 | LSWNSKNPVSNGYWKRKKDD | 100 |
| 101 | ELKIIYNAIKLSEKEGNIFD | 120 |
| 121 | IRDLYKRTLKNIDLLTYSFP | 140 |
| 141 | CQDLSQQGIQKGMKRGSGTR | 160 |
| 161 | SGLLWEIERALDSTEKNDLP | 180 |
| 181 | KYLLMENVGALLNKKNEEEL | 200 |
| 201 | NQWKQKLESLGYQNSIEVLN | 220 |
| 221 | AADFGSSQARRRVFMISTLN | 240 |
| 241 | EFVELPKGDKKPKSIKKVLN | 260 |
| 261 | KIVSEKDILNNLLKYNLTEF | 280 |
| 281 | KKTKSNINKASLIGYSKFNS | 300 |
| 301 | EGYVYDPEFTGPTLTASGAN | 320 |
| 321 | SRIKIKDGSNIRKMNSDETF | 340 |
| 341 | LYMGFDSQDGKRVNEIEFLT | 360 |
| 361 | ENQKIFVCGNSISVEVLEAI | 380 |
| 381 | IDKIGG | 386 |

Another M.SssI, useful in for the present invention includes an enzyme having the amino acid sequence of SEQ ID NO:1 wherein the amino acid at position 343 is isoleucine.

The fusion protein comprises a CRISPR/Cas-like protein or a fragment thereof. The CRISPR/Cas-like protein can be derived from a CRISPR1Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Cse2, Csa5, Csn2, Csm2, Csm3, Csm4, Csm5, Csm6, Cmr1 , Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In one embodiment, the CRISPR/Cas-like protein of the fusion protein is derived from a type II CRISPR/Cas system. In exemplary embodiments, the CRISPR/Cas-like protein of the fusion protein is derived from a Cas9 protein. The Cas9 protein can be from Streptococcus pyogenes, Streptococcus thermophiles, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaroinonas naphthalenivorans, Polaromonas sp., Crocosphaera watsonii, Cyanothece sp., Microcystis aeruginosa, Synechococeus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophiles, Pelotomaculum the rmopropionicum, Acidithiobacillus caldus, Acidithiobacillus ferrooxidans, Allochromatium vinosum, Marinobacter sp., Nitrosococcus halophiles, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In general, CRISPR/Cas proteins comprise at least one RNA recognition and/or RNA binding domain. RNA recognition and/or RNA binding domains interact with the guiding RNA. CRISPR/Cas proteins can also comprise nuclease domains (i.e., DNase or RNase domains), DNA binding domains, helicase domains, RNAse domains, protein-protein interaction domains, dimerization domains, as well as other domains.

The CRISPR/Cas-like protein of the fusion protein can be a wild type CRISPR/Cas protein, a modified CRISPR/Cas protein, or a fragment of a wild type or modified CRISPR/Cas protein. The CRISPR/Cas protein can be modified to increase nucleic acid binding affinity and/or specificity, alter an enzymatic activity, and/or change another property of the protein. For example, nuclease (i.e., DNase, RNase) domains of the CRISPR/Cas protein can be modified, deleted, or inactivated. Alternatively, the CRISPR/Cas protein can be truncated to remove domains that are not essential for the function of the fusion protein. The CRISPR/Cas protein can also be truncated or modified to optimize the activity of the effector domain of the fusion protein.

In some embodiments, the CRISPR/Cas-like protein of the fusion protein can be derived from a wild type Cas9 protein or fragment thereof. In other embodiments, the CRISPR/Cas-like protein of the fusion protein can be derived from modified Cas9 protein. For example, the amino acid sequence of the Cas9 protein can be modified to alter one or more properties (e,g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In general, a Cas9 protein comprises at least two nuclease (i.e., DNase) domains. For example, a Cas9 protein can comprise a RuvC-like nuclease domain and a HNH-like nuclease domain. The RuvC and HIGH domains work together to cut single strands to make a double-stranded break in DNA. (Jinek et al., Science, 337: 816-821). In some embodiments, the Cas9-derived protein can be modified to contain only one functional nuclease domain (either a RuvC-like or a HNH-like nuclease domain).

In other embodiments, both of the RuvC-like nuclease domain and the HNH-like nuclease domain can be modified or eliminated such that the Cas9-derived protein is unable to nick or cleave double stranded nucleic acid. In still other embodiments, all nuclease domains of the Cas9-derived protein can be modified or eliminated such that the Cas9-derived protein lacks all nuclease activity.

In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art. In an exemplary embodiment, the CRISPR/Cas-like protein of the fusion protein is derived from a Cas9 protein in which all the nuclease domains have been inactivated or deleted.

The effector domain of the fusion protein can be an epigenetic modification domain. Preferably the epigenic modification domain is a split. In general, epigenetic modification domains alter gene expression by modifying the histone structure and/or chromosomal structure. Suitable epigenetic modification domains include, without limit, histone acetyltransferase domains, histone deacetylase domains, histone methyltransferase domains, histone demethylase domains, DNA methyltransferase domains, and DNA demethylase domains. As used herein, “DNA methyltransferase” is a protein which is capable of methylating a particular DNA sequence, which particular DNA sequence may be -CpG-. This protein may be a mutated DNA methyltransferase, a wild type DNA methyltransferase, a naturally occurring DNA methyltransferase, a variant of a naturally occurring DNA methyltransferase, a truncated DNA methyltransferase, or a segment of a DNA methyltransferase which is capable of methylating DNA. The DNA methyltransferase may include mammalian DNA methyltransferase, bacterial DNA methyltransferase, M.SssI DNA methyltransferase and other proteins or polypeptides that have the capability of methylating DNA.

In some embodiments the fusion proteins comprise a linker between the first or second fragment of the bifurcated enzyme and a DNA binding domain. The linker is for example is positively charged, negatively charged or polar. The linker is comprised of amino acids and can vary in length from about 5 amino acids to 100 amino acids in length. Preferably, the linker is between about 5 amino acids to 75 amino acids in length. More preferably the about 5 amino acids to 50 amino acids in length. Exemplary linkers include the amino acid sequence (GGGGS)₃, TGGGSGHA or TGGGTSDGGSSETGGSSDTGGSSETGGPGHA.

In some embodiments, the fusion protein further comprises at least one additional domain. Non-limiting examples of suitable additional domains include nuclear localization signals (NLSs), cell-penetrating or translocation domains, and marker domains.

In certain embodiments, the fusion protein can comprise at least one nuclear localization signal. In general, an NLS comprises a stretch of basic amino acids. Nuclear localization signals are known in the art (see, e.g., Lange et al., J. Biol. Chem., 2007, 282:5101-5105). For example, the NLS is from the nucleoplasim protein, SV40, or c-Myc.

In some embodiments the NLS is also the linker.

In some embodiments, the fusion protein can comprise at least one cell-penetrating domain. In one embodiment, the cell-penetrating domain can be a cell-penetrating peptide sequence derived from the HIV-1. TAT protein. a cell-penetrating peptide sequence derived from the human hepatitis B virus. I, Pep-1, VP22, a cell-penetrating peptide from Herpes simplex virus, or a polyarginine peptide sequence. The cell-penetrating domain can be located at the N-terminus, the C-terminal, or in an internal location of the fusion protein.

In still other embodiments, the fusion protein can comprise at least one marker domain. Non-limiting examples of marker domains include fluorescent proteins, purification tags, and epitope tags. In some embodiments, the marker domain can be a fluorescent protein. Non limiting examples of suitable fluorescent proteins include green fluorescent proteins GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1,), blue fluorescent proteins (e.g. EBFP, EBFP2, Azurite, mKalama1, GFPuv, Sapphire, T-sapphire,), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express. DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain can be a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6.times.His, biotin carboxyl carrier protein (BCCP), and calmodulin.

The present disclosure also provides systems comprising at least two fusion proteins according to the invention. In these embodiments, each fusion protein would recognize a different target site (i.e., specified by the protospacer and/or PAM sequence) For example, the guiding RNAs could position the heterodimer to different but closely adjacent sites such that their nuclease domains results in an effective double stranded break in the target DNA. Additionally, each fusion protein would have a split epigenetic modification domain where when associated would form a functional (i.e., active) epigenetic modification domain.

Another aspect of the present disclosure provides nucleic acids encoding any of the fusion proteins or protein dimers described above in sections (I) and (II). The nucleic acid encoding the fusion protein can be RNA or DNA. In one embodiment, the nucleic acid encoding the fusion protein is mRNA. In another embodiment, the nucleic acid encoding the fusion protein is DNA. The DNA encoding the fusion protein can be present in a vector.

The nucleic acid encoding the fusion protein can be codon optimized for efficient translation into protein in the eukaryotic cell or animal of interest. For example, codons can be optimized for expression in humans, mice, rats, hamsters, cows, pigs, cats, dogs, fish, amphibians, plants, yeast, insects, and so forth (see Codon Usage Database at www.kazusa.or.jp/codon/). Programs for codon optimization are available as freeware (e.g., OPTIMIZER or OptimumGene.™.). Commercial codon optimization programs are also available.

In some embodiments, DNA encoding the fusion protein can be operably linked to at least one promoter control sequence. In some iteration, the DNA coding sequence can be operably linked to a promoter control sequence for expression in the eukaryotic cell or animal of interest. The promoter control sequence can be constitutive or regulated. The promoter control sequence can be tissue-specific. Suitable constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED 1)-alpha promoter, ubiquitin promoters, actin promoters, tubulin promoters, immunoglobulin promoters, fragments thereof, or combinations of any of the foregoing. Examples of suitable regulated promoter control sequences include without limit those regulated by heat shock, metals, steroids, antibiotics, or alcohol. Non-limiting examples of tissue specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-.beta. promoter, Mb promoter, NphsI promoter, OG-2 promoter, SP-B promoter, SYN1 promoter, and WASP promoter. The promoter sequence can be wild type or it can be modified for more efficient or efficacious expression. In one exemplary embodiment, the DNA encoding the fusion is operably linked to a CMV promoter for constitutive expression in mammalian cells.

In other embodiments, the sequence encoding the fusion protein can be operably linked to a promoter sequence that is recognized by a phage RNA polymerase for in vitro mRNA synthesis. For example, the promoter sequence can be a T7, T3, or SP6 promoter sequence or a variation of a T7, T3, or SP6 promoter sequence. In an exemplary embodiment, the DNA encoding the fusion protein is operably linked to a T7 promoter for in vitro mRNA synthesis using T7 RNA polymerase.

In alternate embodiments, the sequence encoding the fusion protein can be operably linked to a promoter sequence for in vitro expression of the fusion protein in bacterial or eukaryotic cells. In such embodiments, the expression fusion protein can be purified for use in the methods detailed below in section (IV). Suitable bacterial promoters include, without limit, T7 promoters, lac operon promoters, trp promoters, variations thereof, and combinations thereof. An exemplary bacterial promoter is tac which is a hybrid of trp and lac promoters. Non-limiting examples of suitable eukaryotic promoters are listed above.

In various embodiments, the DNA encoding the fusion protein can be present in a vector. Suitable vectors include plasmid vectors, phagemids, cosmids, artificial/mini-chromosomes, transposons, and viral vectors. In one embodiment, the DNA encoding the fusion protein is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3.sup.rd edition, 2001.

Another aspect of the present disclosure encompasses a method for modifying a chromosomal sequence or regulating expression of a chromosomal sequence in a cell, embryo, or animal. The method comprises introducing into the cell or embryo (a) at least two fusion protein or a nucleic acid encoding the fusion protein, the fusion protein comprising a CRISPR/Cas-like protein or a fragment thereof and an bifurcated effector domain, and (b) at least two guiding RNA or DNA encoding the guiding RNA, wherein the guiding RNA guides the CRISPR/Cas-like protein of the fusion protein to a targeted site in the chromosomal sequence and the effector domain of the fusion protein modifies the chromosomal sequence or regulates expression of the chromosomal sequence.

The fusion protein in conjunction with the guiding RNA is directed to a target site in the chromosomal sequence. The target site has no sequence limitation except that the sequence is immediately followed (downstream) by a consensus sequence. This consensus sequence is also known as a protospacer adjacent motif (PAM). Examples of PAM include, but are not limited to, NGG, NGGNG, and NNAGAAW (wherein N is defined as any nucleotide and W is defined as either A or T). The target site can be in the coding region of a gene, in an intron of a gene, in a control region between genes, etc. The gene can be a protein coding gene or an RNA coding gene.

In some embodiments, the fusion protein or proteins can be introduced into the cell or embryo as an isolated protein. In one embodiment, the fusion protein can comprise at least one cell-penetrating domain, which facilitates cellular uptake of the protein. In other embodiments, an mRNA molecule or molecules encoding the fusion protein or proteins can be introduced into the cell or embryo. In still other embodiments, a DNA molecule or molecules encoding the fusion protein or proteins can be introduced into the cell or embryo. In general, DNA sequence encoding the fusion protein is operably linked to a promoter sequence that will function in the cell or embryo of interest. The DNA sequence can be linear, or the DNA sequence can be part of a vector. In still other embodiments, the fusion protein can be introduced into the cell or embryo as an RNA-protein complex comprising the fusion protein and the guiding RNA.

In alternate embodiments, DNA encoding the fusion protein can further comprise sequence encoding the guiding RNA. In general, the DNA sequence encoding the fusion protein and the guiding RNA is operably linked to appropriate promoter control sequences (such as the promoter control sequences discussed herein for fusion protein and guiding RNA expression) that allow the expression of the fusion protein and the guiding RNA, respectively, in the cell or embryo. The DNA sequence encoding the fusion protein and the guiding RNA can further comprise additional expression control, regulatory, and/or processing sequence(s). The DNA sequence encoding the fusion protein and the guiding RNA can be linear or can be part of a vector.

A guiding RNA interacts with the CRISPR/Cas-like protein of the fusion protein to guide the fusion protein to a specific target site, wherein the effector domain of the fusion protein modifies the chromosomal sequence or regulates expression of the chromosomal sequence.

Each guiding RNA comprises three regions: a first region at the 5′ end that is complementary to the target site in the chromosomal sequence, a second internal region that forms a stem loop structure, and a third 3′ region that remains essentially single-stranded. The first region of each guiding RNA is different such that each guiding RNA guides a fusion protein to a specific target site. The second and third regions of each guiding RNA can be the same in all guiding RNAs.

The first region of the guiding RNA is complementary to the target site in the chromosomal sequence such that the first region of the guiding RNA can base pair with the target site. In various embodiments, the first region of the guiding RNA can comprise from about 10 nucleotides to more than about 25 nucleotides. For example, the region of base pairing between the first region of the guiding RNA and the target site in the chromosomal sequence can be about 4, 5, 6, 7 8, 9, 10, 11, 12, 13, 14, 15, 16, 17, 18, 19, 20, 22, 23, 24, 25, or more than 25 nucleotides in length. In an exemplary embodiment, the first region of the guiding RNA is about 8 or less nucleotides in length.

The guiding RNA also comprises a third region at the 3′ end that remains essentially single-stranded. Thus, the third region has no complementarity to any chromosomal sequence in the cell of interest and has no complementarity to the rest of the guiding RNA. The length of the third region can vary. In general, the third region is more than about 4 nucleotides in length. For example, the length of the third region can range from about 5 to about 30 nucleotides in length.

In another embodiment, the guiding RNA can comprise two separate molecules. The first RNA molecule can comprise the first region of the guiding RNA and one half of the “stem” of the second region of the guiding RNA. The second RNA molecule can comprise the other half of the “stem” of the second region of the guiding RNA and the third region of the guiding RNA. Thus, in this embodiment, the first and second RNA molecules each contain a sequence of nucleotides that are complementary to one another. For example, in one embodiment, the first and second RNA molecules each comprise a sequence (of about 6 to about 20 nucleotides) that base pairs to the other sequence.

In embodiments in which the guiding RNA is introduced into the cell as a DNA molecule, the guiding RNA coding sequence can be operably linked to promoter control sequence for expression of the guiding RNA in the eukaryotic cell. For example, the RNA coding sequence can be operably linked to a promoter sequence that is recognized by RNA polymerase III (Pot III). Examples of suitable Pol III promoters include, but are not limited to, mammalian U6 or H1 promoters. In exemplary embodiments, the RNA coding sequence is linked to a mouse or human U6 promoter. In other exemplary embodiments, the RNA coding sequence is linked to a mouse or human H1 promoter.

The DNA molecule encoding the guiding RNA can be linear or circular. In some embodiments, the DNA sequence encoding the guiding RNA can be part of a vector. Suitable vectors include plasmid vectors, phagemids, cosmids, artificial/mini-chromosomes, transposons, and viral vectors. In an exemplary embodiment, the DNA encoding the RNA-guided endonuclease is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like.

The fusion protein(s) (or nucleic acid(s) encoding the fusion protein(s), the guiding RNA(s) or DNAs encoding the guiding RNAs, can be introduced into a cell or embryo by a variety of means. Typically, the embryo is a fertilized one-cell stage embryo of the species of interest. In sonic embodiments, the cell or embryo is transfected. Suitable transfection methods include calcium phosphate-mediated transfection, nucleofection (or electroporation), cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3.sup.rd edition, 2001). In other embodiments, the molecules are introduced into the cell or embryo by microinjection. For example, the molecules can be injected into the pronuclei of one cell embryos.

The fusion protein(s) (or nucleic acid(s) encoding the fusion protein(s)), the guiding RNA(s) or DNAs encoding the guiding RNAs, can be introduced into the cell or embryo simultaneously or sequentially. The ratio of the fusion protein (or its encoding nucleic acid) to the guiding RNA(s) (or DNAs encoding the guiding RNA), generally will be approximately stoichiometric such that they can form an RNA-protein complex. In one embodiment, the fusion protein and the guiding RNA(s) (or the DNA sequence encoding the fusion protein and the guiding RNA(s)) are delivered together within the same nucleic acid or vector.

The method further comprises maintaining the cell or embryo under appropriate conditions such that the guiding RNA guides the fusion protein to the targeted site in the chromosomal sequence, and the effector domain of the fusion protein modifies the chromosomal sequence or regulates expression of the chromosomal sequence.

In general, the cell is maintained under conditions appropriate for cell growth and/or maintenance. Suitable cell culture conditions are well known in the art and are described, for example, in. Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

An embryo can be cultured in vitro (e.g., in cell culture). Typically, the embryo is cultured at an appropriate temperature and in appropriate media with the necessary O₂/CO₂ ratio to allow the expression of the RNA endonuclease and guiding RNA, if necessary. Suitable non-limiting examples of media include M2, M16, KSOM, BMOC, and HIT media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

A variety of eukaryotic cells are suitable for use in the method. In various embodiments, the cell can be a human cell, a non-human mammalian cell, a non-mammalian vertebrate cell, an invertebrate cell, an insect cell, a plant cell, a yeast cell, or a single cell eukaryotic organism. A variety of embryos are suitable for use in the method. For example, the embryo can be a one cell non-human mammalian embryo. Exemplary mammalian embryos, including one cell embryos, include without limit mouse, rat, hamster, rodent, rabbit, feline, canine, ovine, porcine, bovine, equine, and primate embryos. In still other embodiments, the cell can be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, unipotent stem cells and others. In exemplary embodiments, the cell is a mammalian cell or the embryo is a mammalian embryo.

Non-limiting examples of suitable mammalian cells include Chinese hamster ovary (CHO) cells, baby hamster kidney (BHK) cells; mouse myeloma. NS0 cells, mouse embryonic fibroblast 3T3 cells (NIH3T3), mouse B lymphoma A20 cells; mouse melanoma B16 cells; mouse myoblast C2C12 cells; mouse myeloma SP2/0 cells; mouse embryonic mesenchymal C3H-10T1/2 cells; mouse carcinoma CT26 cells, mouse prostate DuCuP cells; mouse breast EMT6 cells; mouse hepatoma Nepalc1c7 cells; mouse myeloma J5582 cells; mouse epithelial MTD-1A cells; mouse myocardial MyEnd cells; mouse renal RenCa cells; mouse pancreatic RIN-5F cells; mouse melanoma. X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; African green monkey kidney (VERO-76) cells; human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, and human K562 cells. An extensive list of mammalian cell lines may be found in the American Type Culture Collection catalog (ATCC, Manassas, Va.).

Another embodiment of this invention is a method for regulating the expression of a target gene which includes contacting a promoter sequence of the target gene with the chimeric protein described hereinabove, so as to specifically methylate or demethylate the promoter sequence of the target gene thus regulating expression of the target gene. In this embodiment, the target gene may be an endogenous target gene which is native to a cell or a foreign target gene. The foreign gene may be a retroviral target gene or a viral target gene.

The target gene in this embodiment may be associated with a cancer, a central nervous system disorder, a blood disorder, a metabolic disorder, a cardiovascular disorder, an autoimmune disorder, or an inflammatory disorder. The cancer may be acute lymphocytic leukemia, acute myelogenous leukemia, B-cell lymphoma, lung cancer, breast cancer, ovarian cancer, prostate cancer, lymphoma, Hodgkin's disease, malignant melanoma, neuroblastoma, renal cell carcinoma or squamous cell carcinoma. The central nervous system disorder may be Alzheimer's disease, Down's syndrome, Parkinson's disease, Huntington's disease, schizophrenia, or multiple sclerosis. The infectious disease may be cytomegalovirus, herpes simplex virus, human immunodeficiency virus, AIDS, papillomavirus, influenza, candida albicans, mycobacteria, septic shock, or associated with a gram negative bacteria. The blood disorder may be anemia, hemoglobinopathies, sickle cell anemia, or hemophilia. The cardiovascular disorder may be familial hypercholesterolemia, atherosclerosis, or renin/angiotensin control disorder.

The metabolic disorder may be ADA, deficient SCID, diabetes, cystic fibrosis, Gaucher's disease, galactosemia, growth hormone deficiency, inherited emphysema, Lesch-Nyhan disease, liver failure, muscular dystrophy, phenylketonuria, or Tay-Sachs disease. The autoimmune disorder may be arthritis, psoriasis, HIV, or atopic dermatitis. The inflammatory disorder may be acute pancreatitis, irritable bowel syndrome, Chrone's disease or an allergic disorder,

Genes that are overexpressed in cancer cells are also target genes of the subject invention. Inhibiting the expression of these target genes may reduce tumorigenesis and/or metastasis and invasion.

Viruses that establish chronic infections and which are involved in cancer or chronic diseases are also target genes of the subject invention. Virus that have possible target genes include hepatitis C, hepatitis B, varicella, herpes simplex types I and II, Epstein-Barr virus, cytomegalovirus, JC virus and BK virus.

The target gene in this embodiment may be associated with a genetic disorder. Exemplary genetic disorders suitable for treatment with the compositions and methods of the invention include those listed at http://en.wikipedia.org/wiki/List of genetic disorders. (the contents of which is hereby incorporated by reference in its entirety) and include for example 1p36 deletion syndrome, 18p deletion syndrome, 21-hydroxylase deficiency, 47, XXX, see triple X syndrome, 47, XXY, see Klinefelter syndrome, 5-ALA dehydratase-deficient porphyria, see ALA dehydratase deficiency, 5-aminolaevulinic dehydratase deficiency porphyria, see ALA dehydratase deficiency, 5p deletion syndrome, see Cri du chat, 5p-syndrome, see Cri du chat, A-T, see ataxia telangiectasia, AAT, see alpha 1-antitrypsin deficiency, aceruloplasminemia, ACG2, see achondrogenesis type II, ACH, see achondroplasia, Achondrogenesis type II, achondroplasia, Acid beta-glucosidase deficiency, see Gaucher disease type 1, acrocephalosyndactyly (Apert), see Apert syndrome, acrocephalosyndactyly, type V, see Pfeiffer syndrome, Acrocephaly, see Apert syndrome, Acute cerebral Gaucher's disease, see Gaucher disease type 2, acute intermittent porphyria, ACY2 deficiency, see Canavan disease, AD, see Alzheimer's disease Adelaide-type craniosynostosis, see Muenke syndrome, Adenomatous Polyposis Coli, see familial adenomatous polyposis, Adenomatous Polyposis of the Colon see familial adenomatous polyposis ADP, see ALA dehydratase deficiency, adenylosuccinate lyase deficiency, Adrenal gland disorders, see 21-hydroxylase deficiency, Adrenogenital syndrome, see 21-hydroxylase deficiency, Adrenoleukodystrophy, AIP, see acute intermittent porphyria, AIS, see androgen insensitivity syndrome, AKU, see alkaptonuria, ALA dehydratase porphyria, see ALA dehydratase deficiency, ALA-D porphyria, see ALA dehydratase deficiency dehydratase deficiency, Alagille syndrome, Albinism, Alcaptonuria, see alkaptonuria Alexander disease, alkaptonuria, Alkaptonuric ochronosis, see alkaptonuria, alpha 1-antitrypsin deficiency, alpha-1 proteinase inhibitor, see alpha 1-antitrypsin deficiency, alpha-1 related emphysema, see alpha 1-antitrypsin deficiency, Alpha-galactosidase A deficiencysee Fabry disease, ALS, see amyotrophic lateral sclerosis, Alström syndrome, ALX, see Alexander disease, Alzheimer's disease, Amelogenesis imperfecta, Amino levulinic acid dehydratase deficiency, see ALA dehydratase deficiency, Aminoacylase 2 deficiency, see Canavan disease, amyotrophic lateral sclerosis, Anderson-Fabry disease, see Fabry disease androgen insensitivity syndrome, Anemia, Anemia, hereditary sideroblastic, see X-linked sideroblastic anemia, Anemia, splenic, familial, see Gaucher disease, Angelman syndrome Angiokeratoma Corporis Diffusum, see Fabry disease, Angiokeratoma diffuse, see Fabry disease Angiomatosis retinae, see von Hippel-Lindau disease, APC resistance, Leiden type, see factor V Leiden thrombophilia, Apert syndrome, AR deficiency, see androgen insensitivity syndrome, AR-CMT2, see Charcot-Marie-Tooth disease, type 2, Arachnodactyly, see Marfan syndrome ARNSHL, see Nonsyndromic deafness#autosomal recessive, Arthro-ophthalmopathy, hereditary progressive, see Stickler syndrome#COL2A1, Arthrochalasis multiplex congenita, see Ehlers-Danlos syndrome#arthrochalasia type, AS, see Angelman syndrome, Asp deficiency, see Canavan disease, Aspa deficiency, see Canavan disease, Aspartoacylase deficiency see Canavan disease, ataxia telangiectasia, Autism-Dementia-Ataxia-Loss of Purposeful Hand Use syndrome, see Rett syndrome, autosomal dominant juvenile ALS, see amyotrophic lateral sclerosis, type 4, Autosomal dominant opitz G/BBB syndrome, see 22q11.2 deletion syndrome autosomal recessive form of juvenile ALS type 3, see Amyotrophic lateral sclerosis#type 2 Autosomal recessive nonsyndromic hearing loss, see Nonsyndromic deafness#autosomal recessive, Autosomal Recessive Sensorineural Hearing Impairment and Goiter, see Pendred syndrome, AxD, see Alexander disease, Ayerza syndrome, see primary pulmonary hypertension B variant of the Hexosaminidase GM2 gangliosidosis, see Sandhoff disease, BANF, see neurofibromatosis type II, Beare-Stevenson cutis gyrata syndrome, Benign paroxysmal peritonitis, see Mediterranean fever, familial, Benjamin syndrome, beta-thalassemia, BH4 Deficiency, see tetrahydrobiopterin deficiency, Bilateral Acoustic Neurofibromatosis, see neurofibromatosis type II, biotinidase deficiency, bladder cancer, Bleeding disorders see factor V Leiden thrombophilia, Bloch-Sulzberger syndrome, see incontinentia pigmenti, Bloom syndrome, Bone diseases, Bourneville disease, see tuberous sclerosis, Brain diseases, see prion disease, breast cancer, Birt-Hogg-Dubé syndrome, Brittle bone disease, see osteogenesis imperfecta, Broad Thumb-Hallux syndrome, see Rubinstein-Taybi syndrome Bronze Diabetes, see hemochromatosis, Bronzed cirrhosis, see hemochromatosis, Bulbospinal muscular atrophy, X-linked, see Spinal and bulbar muscular atrophy, Burger-Gratz syndrome, see lipoprotein lipase deficiency, familial, CADASIL syndrome, CGD Chronic, granulomatous disorder, Campomelic dysplasia, Canavan disease, Cancer, Cancer Family syndrome, see hereditary nonpolyposis colorectal cancer, Cancer of breast, see breast cancer, Cancer of the bladder, see bladder cancer, Carboxylase Deficiency, Multiple, Late-Onset, see biotinidase deficiency, Cat cry syndrome, see Cri du chat, Caylor cardiofacial syndrome, see 22q11.2 deletion syndrome, Ceramide trihexosidase deficiency, see Fabry disease, Cerebelloretinal Angiomatosis, familial, see von Hippel-Lindau disease, Cerebral arteriopathy, with subcortical infarcts and leukoencephalopathy, see CADASIL syndrome, Cerebral autosomal dominant ateriopathy, with subcortical infarcts and leukoencephalopathy, see CADASIL syndrome, Cerebroatrophic Hyperammonemia, see Rett syndrome, Cerebroside Lipidosis syndrome, see Gaucher disease, CF, see cystic fibrosis, Charcot disease, see amyotrophic lateral sclerosis, Charcot-Marie-Tooth disease, Chondrodystrophia, see achondroplasia, Chondrodystrophy syndrome, see achondroplasia, Chondrodystrophy with sensorineural deafness, see otospondylomegaepiphyseal dysplasia, Chondrogenesis imperfecta, see achondrogenesis, type II, Choreoathetosis self-mutilation hyperuricemia syndrome, see Lesch-Nyhan syndrome, Classic Galactosemia, see galactosemia, Classical Ehlers-Danlos syndrome, see Ehlers-Danlos syndrome#classical type, Classical Phenylketonuria, see phenylketonuria, Cleft lip and palate, see Stickler syndrome, Cloverleaf skull with thanatophoric dwarfism, see Thanatophoric dysplasia#type 2, CLS see Coffin-Lowry syndrome, CMT see Charcot-Marie-Tooth disease, Cockayne syndrome, Coffin-Lowry syndrome, collagenopathy, types II and XI, Colon Cancer, familial Nonpolyposis see hereditary, nonpolyposis colorectal cancer, Colon cancer, familial, see familial adenomatous polyposis Colorectal cancer, Complete HPRT deficiency, see Lesch-Nyhan syndrome, Complete hypoxanthine-guanine phosphoribosyltransferase deficiency, see Lesch-Nyhan syndrome Compression neuropathy, see hereditary neuropathy with liability to pressure palsies, Connective tissue disease, Conotruncal anomaly face syndrome, see 22q11.2 deletion syndrome, Cooley's Anemia, see beta-thalassemia, Copper storage disease, see Wilson's disease, Copper transport disease, see Menkes disease, Coproporphyria, hereditary, see hereditary coproporphyria, Coproporphyrinogen oxidase deficiency, see hereditary coproporphyria, Cowden syndrome CPO deficiency, see hereditary coproporphyria, CPRO deficiency, see hereditary coproporphyria CPX deficiency, see hereditary coproporphyria, Craniofacial dysarthrosis, see Crouzon syndrome, Craniofacial Dysostosis, see Crouzon syndrome, Cri du chat, Crohn's disease, fibrostenosing, Crouzon syndrome, Crouzon syndrome with acanthosis nigricans see Crouzonodermoskeletal syndrome, Crouzonodermoskeletal syndrome, CS see Cockayne syndrome, see Cowden syndrome, Curschmann-Batten-Steinert syndrome, see myotonic dystrophy, cutis gyrata syndrome of Beare-Stevenson, see Beare-Stevenson cutis gyrata syndrome, D-glycerate dehydrogenase deficiency, see hyperoxaluria, primary Dappled metaphysis syndrome, see spondyloepinietaphyseal dysplasia, Strudwick type DAT—Dementia Alzheimer's type, see Alzheimer's disease, Genetic hypercalciuria see Dent's disease, DBMD, see muscular dystrophy, Duchenne and Becker types Deafness with goiter, see Pendred syndrome, Deafness-retinitis pigmentosa syndrome see Usher syndrome, Deficiency disease, Phenylalanine Hydroxylase, see phenylketonuria, Degenerative nerve diseases, de Grouchy syndrome 1, see De Grouchy syndrome, Dejerine-Sottas syndrome, see Charcot-Marie-Tooth disease, Delta-aminolevulinate dehydratase deficiency porphyria, see ALA dehydratase deficiency, Dementia see CADASIL syndrome, demyelinogenic leukodystrophy, see Alexander disease, Dermatosparactic type of Ehlers-Danlos syndrome, see Ehlers-Danlos syndrome#dermatosparaxis type, Dermatosparaxis see Ehlers-Danlos syndrome#dermatosparaxis type, developmental disabilities dHMN, see distal hereditary, motor neuropathy, DHMN-V, see distal hereditary motor neuropathy, DHTR deficiency, see androgen insensitivity syndrome, Diffuse Globoid Body Sclerosis, see Krabbe disease, Di George's syndrome, Dihydrotestosterone receptor deficiency see androgen insensitivity syndrome, distal hereditary motor neuropathy, DM1, see Myotonic dystrophy#type 1, DM2, see Myotonic dystrophy#type 2, DSMAV, see distal spinal muscular atrophy, type V, DSN, see Charcot-Marie-Tooth disease#type 4, DSS, see Charcot-Marie-Tooth disease, type 4, Duchenne/Becker muscular dystrophy, see Muscular dystrophy, Duchenne and Becker type, Dwarf, achondroplastic, see achondroplasia, Dwarf, thanatophoric, see thanatophoric dysplasia, Dwarfism, Dwarfism-retinal atrophy-deafness syndrome, see Cockayne syndrome, dysmyelinogenic leukodystrophy, see Alexander disease, Dystrophia myotonica, see myotonic dystrophy, dystrophia retinae pigmentosa-dysostosis syndrome, see Usher syndrome, Early-Onset familial alzheimer disease (EOFAD), see Alzheimer disease#type 1, see Alzheimer disease#type 3, see Alzheimer disease#type 4, EDS, see Ehlers-Danlos syndrome, Ehlers-Danlos syndrome, Ekman-Lobstein disease, see osteogenesis, imperfecta, Entrapment neuropathy, see hereditary neuropathy with liability to pressure palsies, EPP, see erythropoietic protoporphyria, Erythroblastic anemia, see beta-thalassemia, Erythrohepatic protoporphyria, see erythropoietic protoporphyria, Erythroid 5-aminolevulinate synthetase deficiency, see X-linked sideroblastic anemia, erythropoietic protoporphyria, Eye cancer, see retinoblastoma FA—Friedreich ataxia, see Friedreich's ataxia, FA, see fanconi anemia, Fabry disease, Facial injuries and disorders, factor V Leiden thrombophilia, FALS, see amyotrophic lateral sclerosis, familial acoustic neuroma, see neurofibromatosis type II, familial adenomatous polyposis, familial Alzheimer disease (FAD), see Alzheimer's disease familial amyotrophic lateral sclerosis, see amyotrophic lateral sclerosis, familial dysautonomia, familial fat-induced hypertriglyceridemia, see lipoprotein lipase deficiency, familial, familial hemochromatosis, see hemochromatosis, familial LPL deficiency, see lipoprotein lipase deficiency, familial, familial nonpolyposis colon cancer, see hereditary nonpolyposis colorectal cancer, familial paroxysmal polyserositis, see Mediterranean fever, familial, familial PCT see porphyria cutanea tarda, familial pressure-sensitive neuropathy, see hereditary neuropathy with liability to pressure palsies, familial primary pulmonary hypertension (FPPH), see primary pulmonary hypertension, familial vascular leukoencephalopathy, see CADASIL syndrome FAP, see familial adenomatous polyposis, FD, see familial dysautonomia, Ferrochelatase deficiency, see erythropoietic protoporphyria, ferroportin disease, see Haemochromatosis#type 4 Fever, see Mediterranean fever, familial, FG syndrome, FGFR3-associated coronal synostosis see Muenke syndrome, Fibrinoid degeneration of astrocytes, see Alexander disease, Fibrocystic disease of the pancreas, see cystic fibrosis, FMF, see Mediterranean fever, familial Foiling disease, see phenylketonuria, fra(X) syndrome, see fragile X syndrome, fragile X syndrome, Fragilitas ossium, see osteogenesis imperfecta, FRAXA syndrome see fragile X syndrome, FRDA, see Friedreich's ataxia, Friedreich's ataxia, see Friedreich's ataxia Friedreich's ataxia, FXS, see fragile X syndrome, G6PD deficiency, Galactokinase deficiency disease, see galactosemia, Galactose-1-phosphate uridyl-transferase deficiency disease, see galactosemia, galactosemia, Galactosylceramidase deficiency disease, see Krabbe disease Galactosylceramide lipidosis, see Krabbe disease, galactosylcerebrosidase deficiency, see Krabbe disease, galactosylsphingosine lipidosis, see Krabbe disease, GALC deficiency see Krabbe disease, GALT deficiency, see galactosemia, Gaucher disease, Gaucher-like disease see pseudo-Gaucher disease, G13A deficiency, see Gaucher disease type 1, GD, see Gaucher's disease, Genetic brain disorders, genetic emphysema, see alpha 1-antitrypsin deficiency, genetic hemochromatosis, see hemochromatosis, Giant cell hepatitis, neonatal, see Neonatal emoehromatosis, GLA deficiency, see Fabry disease, Glioblastoma, retinal, see retinoblastoma, Glioma, retinal, see retinoblastoma, globoid cell leukodystrophy (GCL, GLD), see Krabbe disease, globoid cell leukoencephalopathy, see Krabbe disease, Glucocerebrosidase deficiency see Gaucher disease, Glucocerebrosidosis, see Gaucher disease, Glucosyl cerebroside lipidosis, see Gaucher disease, Glucosylceramidase deficiency, see Gaucher disease, Glucosylceramide beta-glucosidase deficiency, see Gaucher disease, Glucosylceramide lipidosis, see Gaucher disease, Glyceric aciduria, see hyperoxaluria, primary, Glycine encephalopathy, see Noriketotic hyperglycinemia, Glycolic aciduria, see hyperoxaluria, primary, GM2 gangliosidosis, type 1, see Tay-Sachs disease, Goiter-deafness syndrome, see Pendred syndrome, Graefe-Usher syndrome, see Usher syndrome, Gronblad-Strandberg syndrome, see pseudoxanthoma elasticum Haemochromatosis, see hemochromatosis, Hallgren syndrome, see Usher syndrome, Harlequin type ichthyosis, Hb S disease, see sickle cell anemia, HCH, see hypochondroplasia, HCP, see hereditary coproporphyria, Head and brain malformations, Hearing disorders and deafness, Hearing problems in children, HEF2A, see hemochromatosis#type 2, HEF2B, see hemochromatosis#type 2, Hematoporphyria, see porphyria, Heme synthetase deficiency see erythropoietic protoporphyria, Hemochromatoses, see hemochromatosis, hemochromatosis hemoglobin M disease, see methemoglobinemia#beta-globin type, Hemoglobin S disease see sickle cell anemia, hemophilia, HEP, see hepatoerythropoietic porphyria, hepatic AGT, deficiency, see hyperoxaluria, primary, hepatoerythropoietic porphyria, Hepatolenticular degeneration syndrome, see Wilson disease, Hereditary arthro-ophthalmopathy, see Stickler syndrome, Hereditary coproporphyria, Hereditary dystopic lipidosis, see Fabry disease, Hereditary hemochromatosis (HHC), see hemochromatosis, Hereditary hemorrhagic telangiectasia (HHT), Hereditary Inclusion Body Myopathy, see skeletal muscle regeneration Hereditary iron-loading anemia, see X-linked sideroblastic anemia, Hereditary motor and sensory neuropathy, see Charcot-Marie-Tooth disease, Hereditary motor neuronopathy, type V, see distal hereditary motor neuropathy, Hereditary multiple exostoses, Hereditary nonpolyposis colorectal cancer, Hereditary periodic fever syndrome, see Mediterranean fever, familial, Hereditary Polyposis Coli, see familial adenomatous polyposis, Hereditary pulmonary emphysema, see alpha 1-antitrypsin deficiency, Hereditary resistance to activated protein C see factor V Leiden thrombophilia, Hereditary sensory and autonomic neuropathy type III see familial dysautonomia, Hereditary spastic paraplegia, see infantile-onset ascending hereditary spastic paralysis, Hereditary spinal ataxia, see Friedreich's ataxia, Hereditary spinal sclerosis, see Friedreich's ataxia, Herrick's anemia, see sickle cell anemia, Heterozygous OSMED, see Weissenbacher-Zweymüller syndrome, Heterozygous otospondylomegaepiphyseal dysplasia, see Weissenbacher-Zweymüller syndrome, HexA deficiency, see Tay-Sachs disease Hexosaminidase A deficiency, see Tay-Sachs disease, Hexosaminidase alpha-subunit deficiency (variant B), see Tay-Sachs disease, HFE-associated hemochromatosis, see hemochromatosis HGPS, see Progeria, Hippel-Lindau disease, see von Hippel-Lindau disease, HLAH see hemochromatosis, HMN V, see distal hereditary motor neuropathy, HMSN, see Charcot-Marie-Tooth disease, HNPCC, see hereditary nonpolyposis colorectal cancer, HNPP see hereditary neuropathy with liability to pressure palsies, homocystinuria, Homogentisic acid oxidase deficiency, see alkaptonuria, Homogentisic acidura, see alkaptonuria, Homozygous porphyria cutanea tarda, see hepatoerythropoietic porphyria, HP1, see hyperoxaluria, primary HP2, see hyperoxaluria, primary, HPA, see hyperphenylalaninemia, HPRT—Hypoxanthine-guanine phosphoribosyltransferase deficiency, see Lesch-Nyhan syndrome, HSAN type III see familial dysautonomia, HSAN3, see familial dysautonomia, HSN-III, see familial dysautonomia, Human dermatosparaxis, see Ehlers-Danlos syndrome#dermatosparaxis type, Huntington's disease, Hutchinson-Gilford progeria syndrome, see progeria, Hyperandrogenism, nonclassic type, due to 21-hydroxylase deficiency, see 21-hydroxylase deficiency, Hyperchylomieronemia, familial, see lipoprotein lipase deficiency, familial, Hyperglycinemia with ketoacidosis and leukopenia, see propionic acidemia, Hyperlipoproteinemia type I see lipoprotein lipase deficiency, familial, hyperoxaluria, primary, hyperphenylalaninaemia see hyperphenylalaninemia, hyperphenylalaninemia, Hypochondrodysplasia, see hypochondroplasia, Hypochondrogenesis, Hypochondroplasia, Hypochromic anemia, see X-linked sideroblastic anemia, Hypoxanthine phosphoribosyltransferse (HPRT) deficiency, see Lesch-Nyhan syndrome, IAHSP, see infantile-onset ascending hereditary spastic paralysis ICF syndrome, see Immunodeficiency, centromere instability and facial anomalies syndrome Idiopathic hemochromatosis, see hemochromatosis, type 3, Idiopathic neonatal hemochromatosis see hemochromatosis, neonatal, Idiopathic pulmonary hypertension, see primary pulmonary, hypertension, immune system disorders, see X-linked severe combined immunodeficiency, Incontinentia pigmenti, Infantile cerebral Gaucher's disease, see Gaucher disease type 2 Infantile Gaucher disease, see Gaucher disease type 2, infantile-onset ascending hereditary spastic paralysis, Infertility, inherited emphysema, see alpha 1-antitrypsin deficiency, inherited tendency to pressure palsies, see hereditary neuropathy with liability to pressure palsies Insley-Astley syndrome, see otospondylomegaepiphyseal dysplasia, Intermittent acute porphyria syndrome, see acute intermittent porphyria, Intestinal polyposis-cutaneous pigmentation syndrome, see Peutz-Jeghers syndrome, IP, see incontinentia pigmenti, Iron storage disorder see hemochromatosis, Isodicentric 15, see isodicentric 15, Isolated deafness, see nonsyndromic deafness, Jackson-Weiss syndrome, JH, see Haemochromatosis#type 2, Joubert syndrome, JPLS, see Juvenile Primary Lateral Sclerosis, juvenile amyotrophic lateral sclerosis, see Amyotrophic lateral sclerosis#type 2, Juvenile gout, choreoathetosis, mental retardation syndrome, see Lesch-Nyhan syndrome, juvenile hyperuricemia syndrome, see Lesch-Nyhan syndrome, JWS, see Jackson-Weiss syndrome, KD, see spinal and bulbar muscular atrophy Kennedy disease, see spinal and bulbar muscular atrophy, Kennedy spinal and bulbar muscular atrophy, see spinal and bulbar muscular atrophy, Kerasin histiocytosis, see Gaudier disease, Kerasin lipoidosis, see Gaucher disease, Kerasin thesaurismosis, see Gaucher disease, ketotic glycinemia, see propionic acidemia, ketotic hyperglycinemia, see propionic acidemia, Kidney diseases, see hyperoxaluria, primary, Klinefelter syndrome, Klinefelter syndrome, see Klinefelter syndrome, Kniest dysplasia, Krabbe disease, Kugelberg-Welander disease, see spinal muscular atrophy, Lacunar dementia, see CADASIL syndrome, Langer-Saldino, achondrogenesis, see achondrogenesis, type II, Langer-Saldino dysplasia, see achondrogenesis, type II, Late-onset Alzheimer disease, see Alzheimer disease#type 2, Late-onset familial Alzheimer disease (AD2), see Alzheimer disease#type 2, late-onset Krabbe disease (LOKD), see Krabbe disease, Learning Disorders, see Learning disability, Lentiginosis, perioral, see Peutz-Jeghers syndrome, Lesch-Nyhan syndrome, Leukodystrophies, leukodystrophy with Rosenthal fibers, see Alexander disease, Leukodystrophy, spongiform, see Canavan disease, LFS, see Li-Fraumeni syndrome, Li-Fraumeni syndrome, Lipase D deficiency, see lipoprotein, lipase deficiency, familial, LIPD deficiency, see lipoprotein lipase deficiency, familial, Lipidosis, cerebroside, see Gaucher disease, Lipidosis, ganglioside, infantile, see Tay-Sachs disease, Lipoid histiocytosis (kerasin type), see Gaucher disease, lipoprotein lipase deficiency, familial, Liver diseases, see galactosemia, Lou Gehrig disease, see amyotrophic lateral sclerosis, Louis-Bar syndrome, see ataxia telangiectasia, Lynch syndrome, see hereditary nonpolyposis colorectal cancer, Lysyl-hydroxylase deficiency, see Ehlers-Danlos syndrome#kyphoscoliosis type, Machado-Joseph disease, see Spinocerebellar ataxia type 3, Male breast cancer, see breast, cancer, Male genital disorders, Malignant neoplasm of breast, see breast cancer, malignant tumor of breast, see breast cancer, Malignant tumor of urinary bladder, see bladder cancer, Mammary cancer, see breast cancer, Marfan syndrome, Marker X syndrome, see fragile X syndrome, Martin-Bell syndrome, see fragile X syndrome, McCune-Albright syndrome, McLeod syndrome, MEDNIK, Mediterranean Anemia, see beta-thalassemia, Mediterranean fever, familial, Mega-epiphyseal dwarfism, see otospondylomegaepiphyseal dysplasia, Menkea syndrome, see Menkes disease, Menkes disease, Mental retardation with osteocartilaginous abnormalities, see Coffin-Lowry syndrome, Metabolic disorders, Metatropic dwarfism, type II, see Kniest dysplasia, Metatropic dysplasia type II, see Kniest dysplasia, Methemoglobinemia#beta-globin type, methylmalonic acidemia, MFS, see Marfan syndrome MHAM, see Cowden syndrome, MK, see Menkes disease, Micro syndrome, Microcephaly MMA, see methylmalonic acidemia, MNK, see Menkes disease, Monosomy 1p36 syndrome, see 1p36 deletion syndrome, Motor neuron disease, amyotrophic lateral sclerosis, see amyotrophic lateral sclerosis, Movement disorders, Mowat-Wilson syndrome, Mucopolysaccharidosis (MPS I), Mucoviscidosis, see cystic fibrosis, Muenke syndrome, Multi-Infarct dementia, see CADASIL syndrome, Multiple carboxylase deficiency, late-onset, see biotinidase deficiency, Multiple hamartoma syndrome, see Cowden syndrome, Multiple neurofibromatosis, see neurofibromatosis, Muscular dystrophy, Muscular dystrophy, Duchenne and Becker type, Myotonia atrophica, see myotonic dystrophy, Myotonia dystrophica, see myotonic dystrophy, myotonic dystrophy, Nance-Insley syndrome, see otospondylomegaepiphyseal dysplasia, Nance-Sweeney chondrodysplasia, see otospondylomegaepiphyseal dysplasia, NBIA1, see pantothenate kinase-associated neurodegeneration, Neill-Dingwall syndrome, see Cockayne syndrome, Neuroblastoma, retinal see retinoblastoma, Neurodegeneration with brain iron accumulation type 1, see pantothenate kinase-associated neurodegeneration, Neurofibromatosis type I, Neurofibromatosis type II, Neurologic diseases, Neuromuscular disorders, neuronopathy, distal hereditary motor, type V, see distal hereditary, motor neuropathy, neuronopathy, distal hereditary motor, with pyramidal features, see Amyotrophic lateral sclerosis#type 4, Niemann-Pick, see Niemann-Pick disease Noack syndrome, see Pfeiffer syndrome, Nonketotic hyperglycinemia, see Glycine encephalopathy, Non-neuronopathic Gaucher disease, see Gaucher disease type 1, Non-phenylketonuric hyperphenylalaninemia, see tetrahydrobiopterin deficiency, nonsyndromic deafness, Noonan syndrome, Norrbottnian Gaucher disease, see Gaucher disease type 3 Ochronosis, see alkaptonuria, Ochronotic arthritis, see alkaptonuria, Ogden syndrome, OI, see osteogenesis imperfecta, Osler-Weber-Rendu disease, see Hereditary hemorrhagic telangiectasia, OSMED, see otospondylomegaepiphyseal dysplasia, osteogenesis imperfecta Osteopsathyrosis, see osteogenesis imperfecta, Osteosclerosis congenita, see achondroplasia Oto-spondylo-megaepiphyseal dysplasia, see otospondylomegaepiphyseal dysplasia otospondylomegaepiphyseal dysplasia, Oxalosis, see hyperoxaluria, primary Oxaluria, primary, see hyperoxaluria, primary, pantothenate kinase-associated neurodegeneration Patau Syndrome (Trisomy 13), PBGD deficiency, see acute intermittent porphyria, PCC deficiency, see propionic acidemia, PCT, see porphyria cutanea tarda, PDM, see Myotonic dystrophy#type 2, Pendred syndrome, Periodic disease, see Mediterranean fever, familial Periodic peritonitis, see Mediterranean fever, familial, Periorificial lentiginosis syndrome see Peutz-Jeghers syndrome, Peripheral nerve disorders, see familial dysautonomia, Peripheral neurofibromatosis, see neurofibromatosis type I, Peroneal muscular atrophy, see Charcot-Marie-Tooth disease, peroxisomal alanine:glyoxylate aminotransferase deficiency, see hyperoxaluria, primary, Peutz-Jeghers syndrome, Pfeiffer syndrome, Phenylalanine hydroxylase deficiency disease, see phenylketonuria, phenylketonuria, Pheochromocytoma, see von Hippel-Lindau disease, Pierre Robin syndrome with fetal chondrodysplasia, see Weissenbacher-Zweymüller syndrome, Pigmentary cirrhosis, see hemochromatosis, PJS, see Peutz-Jeghers syndrome, PKAN see pantothenate kinase-associated neurodegeneration, PKU see phenylketonuria Plumboporphyria, see ALA deficiency porphyria, PMA see Charcot-Marie-tooth disease, Polycystic kidney disease, polyostotic fibrous dysplasia, see McCune-Albright syndrome polyposis coli, see familial adenomatous polyposis, polyposis, hamartomatous intestinal see Peutz-Jeghers syndrome, polyposis, intestinal, II, see Peutz-Jeghers syndrome, polyps-and-spots syndrome, see Peutz-Jeghers syndrome, Porphobilinogen synthase deficiency see ALA deficiency porphyria, porphyria, porphyrin disorder, see porphyria, PPH see primary pulmonary hypertension, PPOX deficiency, see variegate porphyria, Prader-Labhart-Willi syndrome, see Prader-Willi syndrome, Prader-Willi syndrome presenile and senile dementia see Alzheimer's disease, Primary ciliary dyskinesia (PCD), primary hemochromatosis see hemochromatosis, primary hyperuricemia syndrome see Lesch-Nyhan syndrome, primary pulmonary hypertension, primary senile degenerative dementia see Alzheimer's disease, procollagen type EDS VII, mutant see Ehlers-Danlos syndrome#arthrochalasia type, progeria see Hutchinson Gilford Progeria Syndrome, Progeria-like syndrome see Cockayne syndrome, progeroid nanism see Cockayne syndrome, progressive chorea, chronic hereditary (Huntington) see Huntington's disease, progessively deforming osteogenesis imperfecta with normal sclerae see Osteogenesis imperfecta#Type III, PROMM see Myotonic dystrophy#type 2 propionic acidemia, propionyl-CoA carboxylase deficiency see propionic acidemia, protein C deficiency, protein S deficiency, protoporphyria, see erythropoietic protoporphyria, protoporphyrinogen oxidase deficiency see variegate porphyria, proximal myotonic dystrophy see Myotonic dystrophy#type 2, proximal myotonic myopathy see Myotonic dystrophy#type 2, pseudo-Gaucher disease, pseudoxanthoma elasticum, psychosine lipidosis see Krabbe disease, pulmonary arterial hypertension see primary pulmonary hypertension, pulmonary hypertension see primary pulmonary hypertension, PWS see Prader-Willi syndrome, PXE—pseudoxanthoma elasticum see pseudoxanthoma elasticum, Rb see retinoblastoma, Recklinghausen disease, nerve see neurofibromatosis type I, Recurrent polyserositis, see Mediterranean fever, familial, Retinal disorders, Retinitis pigmentosa-deafness syndrome see Usher syndrome, Retinoblastoma Rett syndrome, RFALS type 3 see Amyotrophic lateral sclerosis#type 2, Ricker syndrome see Myotonic dystrophy#type 2, Riley-Day syndrome see familial dysautonomia, Roussy-Levy syndrome see Charcot-Marie-Tooth disease, RSTS see Rubinstein-Taybi syndrome, RTS see Rett syndrome, see Rubinstein-Taybi syndrome, RTT see Rett syndrome, Rubinstein-Taybi syndrome, Sack-Barabas syndrome see Ehlers Danlos syndrome, vascular type, SADDAN, sarcoma family syndrome of Li and Fraumeni see Li-Fraumeni syndrome, sarcoma, breast, leukemia, and adrenal gland (SBLA) syndrome see Li-Fraumeni syndrome, SBLA syndrome see Li-Fraumeni syndrome, SBMA see spinal and bulbar musclular atrophy, SCD see sickle cell anemia, Schwannoma, acoustic, bilateral see neurofibromatosis type II Schwartz-Jampel syndrome, SCIDXI see X-linked severe combined immunodeficiency, SDAT see Alzheimer's disease, SED congenita see spondyloepiphyseal dysplasia congenita, SED Strudwick see spondyloepimetaphyseal dysplasia, Strudwick type, SEDc see spondyloepiphyseal dysplasia congenita, SEMD, Strudwick type see spondyloepimetaphyseal dysplasia, Strudwick type, senile dementia see Alzheimer disease#type 2, severe achondroplasia with developmental delay and acanthosis nigricans see SADDAN, Shprintzen syndrome see 22q11.2 deletion syndrome, sickle cell anemia, Siderius X-linked mental retardation syndrome caused by mutations in the PHF8 gene, skeleton-skin-brain syndrome see SADDAN, Skin pigmentation disorders, SMA see spinal muscular atrophy, SMED, Strudwick type see spondyloepimetaphyseal dysplasia, Strudwick type SMED, type I see spondyloepimetaphyseal dysplasia, Strudwick type, Smith-Lemli-Opitz syndrome, Smith Magenis Syndrome, South-African genetic porphyria see variegate porphyria spastic paralysis, infantile onset ascending see infantile-onset ascending hereditary spastic paralysis, Speech and communication disorders, sphingolipidosis, Tay-Sachs see Tay-Sachs disease, spinal and bulbar muscular atrophy, spinal muscular atrophy, spinal muscular atrophy, distal type V see distal hereditary motor neuropathy, spinal muscular atrophy, distal, with upper limb predominance see distal hereditary motor neuropathy, spinocerebellar ataxia, spondyloepimetaphyseal dysplasia, Strudwick type, spondyloepiphyseal dysplasia congenita spondyloepiphyseal dysplasia, see collagenopathy, types II and XI, spondylometaepiphyseal dysplasia congenita, Strudwick type see spondyloepimetaphyseal dysplasia, Strudwick type spondylometaphyseal dysplasia (SMD) see spondyloepimetaphyseal dysplasia, Strudwick type spondylometaphyseal dysplasia, Strudwick type see spondyloepimetaphyseal dysplasia, Strudwick type spongy degeneration of central nervous system see Canavan disease spongy degeneration of the brain, see Canavan disease spongy degeneration of white matter in infancy, see Canavan disease sporadic primary pulmonary hypertension see primary pulmonary hypertension, SSB syndrome see SADDAN, steely hair syndrome see Menkes disease, Steinert disease see myotonic dystrophy, Steinert myotonic dystrophy syndrome see myotonic dystrophy Stickler syndrome, stroke see CADASIL syndrome, Strudwick syndrome see spondyloepimetaphyseal dysplasia, Strudwick type, subacute neuronopathic Gaucher disease see Gaucher disease type 3, Swedish genetic porphyria see acute intermittent porphyria, Swedish porphyria see acute intermittent porphyria, Swiss cheese cartilage dysplasia see Kniest dysplasia, Tay-Sachs disease, TD-thanatophoric dwarfism see thanatophoric dysplasia TD with straight femurs and cloverleaf skull see thanatophoric dysplasia#Type 2, Telangiectasia, cerebello-oculocutaneous see ataxia telangiectasia, Testicular feminization syndrome see androgen insensitivity syndrome, tetrahydrobiopterin deficiency, TFM—testicular feminization syndrome see androgen insensitivity syndrome, thalassemia intermedia see beta-thalassemia, Thalassemia Major see beta-thalassemia, thanatophoric dysplasia Thrombophilia due to deficiency of cofactor for activated protein C, Leiden type see factor V Leiden thrombophilia, Thyroid disease, Tomaculous neuropathy see hereditary neuropathy with liability to pressure palsies, Total HPRT deficiency see Lesch-Nyhan syndrome, Total hypoxanthine-guanine phosphoribosyl transferase deficiency see Lesch-Nyhan syndrome, Treacher Collins syndrome, Trias fragilitis ossium see osteogenesis imperfecta#Type I, triple X syndrome, Triplo X syndrome see triple X syndrome, Trisomy 21 see Down syndrome, Trisomy X see triple X syndrome, Troisier-Hanot-Chauffard syndrome see hemochromatosis, TSD see Tay-Sachs disease, Turner's syndrome see Turner syndrome, Turner-like syndrome see Noonan syndrome, Type 2 Gaucher disease see Gaucher disease type 2, Type 3 Gaucher disease see Gaucher disease type 3, UDP-galactose-4-epimerase deficiency disease see galactosemia, UDP glucose 4-epimerase deficiency disease see galactosemia, UDP glucose hexose-1-phosphate uridylyitransferase deficiency see galactosemia, Undifferentiated deafness see nonsyndromic deafness, UPS deficiency see acute intermittent porphyria, Urinary bladder cancer see bladder cancer, UROD deficiency see porphyria cutanea tarda, Uroporphyrinogen decarboxylase deficiency see porphyria cutanea tarda, Uroporphyrinogen synthase deficiency see acute intermittent porphyria, Usher syndrome, UTP hexose-1-phosphate uridylyltransferase deficiency see galactosemia, Van Bogaert-Bertrand syndrome see Canavan disease, Van der Hoeve syndrome see osteogenesis imperfecta#Type I, variegate porphyria, Velocardiofacial syndrome see 22q11.2 deletion syndrome, VHL syndrome see von Hippel-Lindau disease, Vision impairment and blindness see Alström syndrome, Von Bogaert-Bertrand disease see Canavan disease, von Hippel-Lindau disease, Von Recklenhausen-Applebaum disease see hemochromatosis, von Recklinghausen disease see neurofibromatosis type I, VP see variegate porphyria, Vrolik disease see osteogenesis imperfecta, Waardenburg syndrome, Warburg Sjo Fledelius Syndrome see Micro syndrome, WD see Wilson disease, Weissenbacher-Zweymüller syndrome, Werdnig-Hoffmann disease see spinal muscular atrophy, Williams Syndrome, Wilson disease, Wilson's disease see Wilson disease, Wolf-Hirschhorn syndrome, Wolff Periodic disease see Mediterranean fever, familial WZS see Weissenbacher-Zweymüller syndrome, Xeroderma pigmentosum, X-linked mental retardation and macroorchidism see fragile X syndrome, X-linked primary hyperuricemia see Lesch-Nyhan syndrome, X-linked severe combined immunodeficiency, X-linked sideroblastic anemia, X-linked spinal-bulbar muscle atrophy, see spinal and bulbar muscular atrophy, X-linked uric aciduria enzyme defect see Lesch-Nyhan syndrome, X-SCID see X-linked severe combined immunodeficiency, XLSA see X-linked sideroblastic anemia XSCID see X-linked severe combined immunodeficiency, XXX syndrome see triple X syndrome, XXXX syndrome see 48, XXXX, XXXXX syndrome see 49, XXXXX XXY syndrome see Klinefelter syndrome, XXY trisomy see Klinefelter syndrome, XYY syndrome see 47, XYY syndrome.

Any disease with a “P” for point mutation is a candidate disease that can be corrected by editing. Diseases with “D” or “C” (deletion of a full gene or chromosome, respectively) are less likely candidates for correction by gene editing due to replacement. Diseases with “T” (Trinucleotide repeat diseases) are possible candidates for gene editing through deletion of the repetitive DNA without replacement of corrective sequence.

All of these categories of genetic diseases can be treated through epigenetic approaches according to the methods of the invention. By directing the epigenetic modifying enzymes to sequences that are not causal to the disease. If up or down modulation of these non-disease causing genes is beneficial in palliating disease, these genes can be considered targets for epigenetic induction or repression therapy.

Definitions

Before describing the invention in detail, it is to be understood that this invention is not limited to particular biological systems or cell types. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes combinations of two or more cells, or entire cultures of cells; reference to “a polynucleotide” includes, as a practical matter, many copies of that polynucleotide. Unless defined herein and below in the reminder of the specification, all technical and scientific terms used herein have the same meaning as commonly understood by one of ordinary skill in the art to which the invention pertains.

As used herein, “DNA binding protein portion” is a segment of a DNA binding protein or polypeptide capable of specifically binding to a particular DNA sequence. The binding is specific to a particular DNA sequence site. The DNA binding protein portion may include a truncated segment of a DNA binding protein or a fragment of a DNA binding protein.

As used herein, “binds sufficiently close” means the contacting of a DNA molecule by a protein at a position on the DNA molecule near enough to a predetermined methylation site on the DNA molecule to allow proper functioning of the protein and allow specific methylation of the predetermined methylation site.

As used herein, “a promoter sequence of a target gene” is at least a portion of a non-coding DNA sequence which directs the expression of the target gene. The portion of the non-coding DNA sequence may be in the 5′-prime direction or in the 3′-prime direction from the coding region of the target gene. The portion of the non-coding DNA sequence may be located in an intron of the target gene.

The promoter sequence of the target gene may be a 5′ long terminal repeat sequence of a human immunodeficiency virus-1 proviral DNA. The target gene may be a retroviral gene, an adenoviral gene, a foamy viral gene, a parvo viral gene, a foreign gene expressed in a cell, an overexpressed gene, or a misexpressed gene.

As used herein “specifically methylate” means to bond a methyl group to a methylation site in a DNA sequence, which methylation site may be -CpG-, wherein the methylation is restricted to particular methylation site(s) and the methylation is not random.

As used herein, the terms “polynucleotide,” “nucleic acid,” “oligonucleotide,” “oligomer,” “oligo” or equivalent terms, refer to molecules that comprises a polymeric arrangement of nucleotide base monomers, where the sequence of monomers defines the polynucleotide. Polynucleotides can include polymers of deoxyribonucleotides to produce deoxyribonucleic acid (DNA), and polymers of ribonucleotides to produce ribonucleic acid (RNA). A polynucleotide can be single- or double-stranded. When single stranded, the [polynucleotide can correspond to the sense or antisense strand of a gene. A single-stranded polynucleotide can hybridize with a complementary portion of a target polynucleotide to form a duplex, which can be a homoduplex or a heteroduplex.

The length of a polynucleotide is not limited in any respect. Linkages between nucleotides can be intemucleotide-type phosphodiester linkages, or any other type of linkage. A polynucleotide can be produced by biological means (e.g., enzymatically), either in vivo (in a cell) or in vitro (in a cell-free system). A polynucleotide can be chemically synthesized using enzyme-free systems. A polynucleotide can be enzymatically extendable or enzymatically non-extendable.

By convention, polynucleotides that are formed by 3′-5′ phosphodiester linkages (including naturally occurring polynucleotides) are said to have 5′-ends and 3′-ends because the nucleotide monomers that are incorporated into the polymer are joined in such a manner that the 5′ phosphate of one mononucleotide pentose ring is attached to the 3′ oxygen (hydroxyl) of its neighbor in one direction via the phosphodiester linkage. Thus, the 5′-end of a polynucleotide molecule generally has a free phosphate group at the 5′ position of the pentose ring of the nucleotide, while the 3′ end of the polynucleotide molecule has a free hydroxyl group at the 3′ position of the pentose ring. Within a polynucleotide molecule, a position that is oriented 5′ relative to another position is said to be located “upstream,” while a position that is 3′ to another position is said to be “downstream.” This terminology reflects the fact that polymerases proceed and extend a polynucleotide chain in a 5′ to 3′ fashion along the template strand. Unless denoted otherwise, whenever a polynucleotide sequence is represented, it will be understood that the nucleotides are in 5′ to 3′ orientation from left to right.

As used herein, it is not intended that the term “polynucleotide” be limited to naturally occurring polynucleotide structures, naturally occurring nucleotides sequences, naturally occurring backbones or naturally occurring intemucleotide linkages. One familiar with the art knows well the wide variety of polynucleotide analogues, unnatural nucleotides, non-natural phosphodiester bond linkages and internucleotide analogs that find use with the invention.

As used herein, the expressions “nucleotide sequence,” “sequence of a polynucleotide,” “nucleic acid sequence,” “polynucleotide sequence”, and equivalent or similar phrases refer to the order of nucleotide monomers in the nucleotide polymer. By convention, a nucleotide sequence is typically written in the 5′ to 3′ direction. Unless otherwise indicated, a particular polynucleotide sequence of the invention optionally encompasses complementary sequences, in addition to the sequence explicitly indicated.

As used herein, the term “gene” generally refers to a combination of polynucleotide elements, that when operatively linked in either a native or recombinant manner, provide some product or function. The term “gene” is to be interpreted broadly, and can encompass mRNA, cDNA, eRNA and genomic DNA forms of a gene. In some uses, the term “gene” encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some aspects, genes do not encode a polypeptide, for example, ribosomal RNA genes (rRNA) and transfer RNA (tRNA) genes. In some aspects, the term “gene” includes not only the transcribed sequences, but in addition, also includes non-transcribed regions including upstream and downstream regulatory regions, enhancers and promoters. The term “gene” encompasses mRNA, cDNA and genomic forms of a gene.

In some aspects, the genomic form or genomic clone of a gene includes the sequences of the transcribed mRNA, as well as other non-transcribed sequences which lie outside of the transcript. The regulatory regions which lie outside the mRNA transcription unit are termed 5′ or 3′ flanking sequences. A functional genomic form of a gene typically contains regulatory elements necessary, and sometimes sufficient, for the regulation of transcription. The term “promoter” is generally used to describe a DNA region, typically but not exclusively 5′ of the site of transcription initiation, sufficient to confer accurate transcription initiation. In some aspects, a “promoter” also includes other cis-acting regulatory elements that are necessary for strong or elevated levels of transcription, or confer inducible transcription. In some embodiments, a promoter is constitutively active, while in alternative embodiments, the promoter is conditionally active (e.g., where transcription is initiated only under certain physiological conditions).

Generally, the term “regulatory element” refers to any cis-acting genetic element that controls some aspect of the expression of nucleic acid sequences. In some uses, the term “promoter” comprises essentially the minimal sequences required to initiate transcription. In some uses, the term “promoter” includes the sequences to start transcription, and in addition, also include sequences that can upregulate or downregulate transcription, commonly termed “enhancer elements” and “repressor elements,” respectively.

Specific DNA regulatory elements, including promoters and enhancers, generally only function within a class of organisms. For example, regulatory elements from the bacterial genome generally do not function in eukaryotic organisms. However, regulatory elements from more closely related organisms frequently show cross functionality. For example, DNA regulatory elements from a particular mammalian organism, such as human, will most often function in other mammalian species, such as mouse. Furthermore, in designing recombinant genes that will function across many species, there are consensus sequences for many types of regulatory elements that are known to function across species, e.g., in all mammalian cells, including mouse host cells and human host cells.

As used herein, the expressions “in operable combination,” “in operable order,” “operatively linked,” “operatively joined” and similar phrases, when used in reference to nucleic acids, refer to the operational linkage of nucleic acid sequences placed in functional relationships with each other. For example, an operatively linked promoter, enhancer elements, open reading frame, 5′ and 3′ UTR, and terminator sequences result in the accurate production of an RNA molecule. In some aspects, operatively linked nucleic acid elements result in the transcription of an open reading frame and ultimately the production of a polypeptide (i.e., expression of the open reading frame).

As used herein, the term “genome” refers to the total genetic information or hereditary material possessed by an organism (including viruses), i.e., the entire genetic complement of an organism or virus. The genome generally refers to all of the genetic material in an organism's chromosome(s), and in addition, extra-chromosomal genetic information that is stably transmitted to daughter cells (e.g., the mitochondrial genome). A genome can comprise RNA or DNA. A genome can be linear (mammals) or circular (bacterial). The genomic material typically resides on discrete units such as the chromosomes.

As used herein, a “polypeptide” is any polymer of amino acids (natural or unnatural, or a combination thereof), of any length, typically but not exclusively joined by covalent peptide bonds. A polypeptide can be from any source, e.g., a naturally occurring polypeptide, a polypeptide produced by recombinant molecular genetic techniques, a polypeptide from a cell, or a polypeptide produced enzymatically in a cell-free system. A polypeptide can also be produced using chemical (non-enzymatic) synthesis methods. A polypeptide is characterized by the amino acid sequence in the polymer. As used herein, the term “protein” is synonymous with polypeptide. The term “peptide” typically refers to a small polypeptide, and typically is smaller than a protein. Unless otherwise stated, it is not intended that a polypeptide be limited by possessing or not possessing any particular biological activity.

As used herein, the expressions “codon utilization” or “codon bias” or “preferred codon utilization” or the like refers, in one aspect, to differences in the frequency of occurrence of any one codon from among the synonymous codons that encode for a single amino acid in protein-coding DNA (where many amino acids have the capacity to be encoded by more than one codon). In another aspect, “codon use bias” can also refer to differences between two species in the codon biases that each species shows. Different organisms often show different codon biases, where preferences for which codons from among the synonymous codons are favored in that organism's coding sequences.

As used herein, the terms “vector,” “vehicle,” “construct” and “plasmid” are used in reference to any recombinant polynucleotide molecule that can be propagated and used to transfer nucleic acid segment(s) from one organism to another. Vectors generally comprise parts which mediate vector propagation and manipulation (e.g., one or more origin of replication, genes imparting drug or antibiotic resistance, a multiple cloning site, operably linked promoter/enhancer elements which enable the expression of a cloned gene, etc.). Vectors are generally recombinant nucleic acid molecules, often derived from bacteriophages, or plant or animal viruses. Plasmids and cosmids refer to two such recombinant vectors. A “cloning vector” or “shuttle vector” or “subcloning vector” contain operably linked parts that facilitate subcloning steps (e.g., a multiple cloning site containing multiple restriction endonuclease target sequences). A nucleic acid vector can be a linear molecule, or in circular form, depending on type of vector or type of application. Some circular nucleic acid vectors can be intentionally linearized prior to delivery into a cell.

As used herein, the term “expression vector” refers to a recombinant vector comprising operably linked polynucleotide elements that facilitate and optimize expression of a desired gene (e.g., a gene that encodes a protein) in a particular host organism (e.g., a bacterial expression vector or mammalian expression vector). Polynucleotide sequences that facilitate gene expression can include, for example, promoters, enhancers, transcription termination sequences, and ribosome binding sites.

As used herein, the term “host cell” refers to any cell that contains a heterologous nucleic acid. The heterologous nucleic acid can be a vector, such as a shuttle vector or an expression vector. In some aspects, the host cell is able to drive the expression of genes that are encoded on the vector. In some aspects, the host cell supports the replication and propagation of the vector. Host cells can be bacterial cells such as E. coli, or mammalian cells (e.g., human cells or mouse cells). When a suitable host cell (such as a suitable mouse cell) is used to create a stably integrated cell line, that cell line can be used to create a complete transgenic organism.

Methods (i.e., means) for delivering vectors/constructs or other nucleic acids (such as in vitro transcribed RNA) into host cells such as bacterial cells and mammalian cells are well known to one of ordinary skill in the art, and are not provided in detail herein. Any method for nucleic acid delivery into a host cell finds use with the invention.

For example, methods for delivering vectors or other nucleic acid molecules into bacterial cells (termed transformation) such as Escherichia coli are routine, and include electroporation methods and transformation of E. coli cells that have been rendered competent by previous treatment with divalent cations such as CaCl₂.

Methods for delivering vectors or other nucleic acid (such as RNA) into mammalian cells in culture (termed transfection) are routine, and a number of transfection methods find use with the invention. These include but are not limited to calcium phosphate precipitation, electroporation, lipid-based methods (liposomes or lipoplexes) such as Transfectamine.®, (Life Technologies.™.) and TransFectin.™. (Bio-Rad Laboratories), cationic polymer transfections, for example using DEAE-dextran, direct nucleic acid injection, biolistic particle injection, and viral transduction using engineered viral carriers (termed transduction, using e.g., engineered herpes simplex virus, adenovirus, adeno-associated virus, vaccinia virus, Sindbis virus), and sonoporation. Any of these methods find use with the invention,

As used herein, the term “recombinant” in reference to a nucleic acid or polypeptide indicates that the material (e.g., a recombinant nucleic acid, gene, polynucleotide, polypeptide, etc.) has been altered by human intervention. Generally, the arrangement of parts of a recombinant molecule is not a native configuration, or the primary sequence of the recombinant polynucleotide or polypeptide has in some way been manipulated. A naturally occurring nucleotide sequence becomes a recombinant polynucleotide if it is removed from the native location from which it originated (e.g., a chromosome), or if it is transcribed from a recombinant DNA construct. A gene open reading frame is a recombinant molecule if that nucleotide sequence has been removed from it natural context and cloned into any type of nucleic acid vector (even if that ORF has the same nucleotide sequence as the naturally occurring gene). Protocols and reagents to produce recombinant molecules, especially recombinant nucleic acids, are well known to one of ordinary skill in the art. In some embodiments, the term “recombinant cell line” refers to any cell line containing a recombinant nucleic acid, that is to say, a nucleic acid that is not native to that host cell.

As used herein, the terms “heterologous” or “exogenous” as applied to polynucleotides or polypeptides refers to molecules that have been rearranged or artificially supplied to a biological system and are not in a native configuration (e.g., with respect to sequence, genomic position or arrangement of parts) or are not native to that particular biological system. These terms indicate that the relevant material originated from a source other than the naturally occurring source, or refers to molecules having a non-natural configuration, genetic location or arrangement of parts. The terms “exogenous” and “heterologous” are sometimes used interchangeably with “recombinant.”

As used herein, the terms “native” or “endogenous” refer to molecules that are found in a naturally occurring biological system, cell, tissue, species or chromosome under study. A “native” or “endogenous” gene is a generally a gene that does not include nucleotide sequences other than nucleotide sequences with which it is normally associated in nature (e.g., a nuclear chromosome, mitochondrial chromosome or chloroplast chromosome). An endogenous gene, transcript or polypeptide is encoded by its natural locus, and is not artificially supplied to the cell.

As used herein, the term “marker” most generally refers to a biological feature or trait that, when present in a cell (e.g., is expressed), results in an attribute or phenotype that visualizes or identifies the cell as containing that marker. A variety of marker types are commonly used, and can be for example, visual markers such as color development, e.g., lacZ complementation (.beta.-galactosidase) or fluorescence, e.g., such as expression of green fluorescent protein (GPP) or GFP fusion proteins, RFP, BFP, selectable markers, phenotypic markers (growth rate, cell morphology, colony color or colony morphology, temperature sensitivity), auxotrophic markers (growth requirements), antibiotic sensitivities and resistances, molecular markers such as biomolecules that are distinguishable by antigenic sensitivity (e.g., blood group antigens and histocompatibility markers), cell surface markers (for example H2KK), enzymatic markers, and nucleic acid markers, for example, restriction fragment length polymorphisms (RFLP), single nucleotide polymorphism (SNP) and various other amplifiable genetic polymorphisms.

As used herein, the expressions “selectable marker” or “screening marker” or “positive selection marker” refer to a marker that, when present in a cell, results in an attribute or phenotype that allows selection or segregated of those cells from other cells that do not express the selectable marker trait. A variety of genes are used as selectable markers, e.g., genes encoding drug resistance or auxotrophic rescue are widely known. For example, kanamycin (neomycin) resistance can be used as a trait to select bacteria that have taken up a plasmid carrying a gene encoding for bacterial kanamycin resistance (e.g., the enzyme neomycin phosphotransferase II). Non-transfected cells will eventually die off when the culture is treated with neomycin or similar antibiotic.

A similar mechanism can also be used to select for transfected mammalian cells containing a vector carrying a gene encoding for neomycin resistance (either one of two aminoglycoside phosphotransferase genes; the neo selectable marker). This selection process can be used to establish stably transfected mammalian cell lines. Geneticin (G418) is commonly used to select the mammalian cells that contain stably integrated copies of the transfected genetic material.

As used herein, the expressions “negative selection” or “negative screening marker” refers to a marker that, when present (e.g., expressed, activated, or the like) allows identification of a cell that does not comprise a selected property or trait (e.g., as compared to a cell that does possess the property or trait).

A wide variety of positive and negative selectable markers are known for use in prokaryotes and eukaryotes, and selectable marker tools for plasmid selection in bacteria and mammalian cells are widely available. Bacterial selection systems include, for example but not limited to, ampicillin resistance (.beta.-lactamase), chloramphenicol resistance, kanamycin resistance (aminoglycoside phosphotransferases), and tetracycline resistance. Mammalian selectable marker systems include, for example but not limited to, neomycin/G418 (neomycin phosphotransferase II), methotrexate resistance (dihydropholate reductase; DHFR), hygromycin-B resistance (hygromycin-B phosphotransferase), and blasticidin resistance (blasticidin S deaminase).

As used herein, the term “reporter” refers generally to a moiety, chemical compound or other component that can be used to visualize, quantitate or identify desired components of a system of interest. Reporters are commonly, but not exclusively, genes that encode reporter proteins. For example, a “reporter gene” is a gene that, when expressed in a cell, allows visualization or identification of that cell, or permits quantitation of expression of a recombinant gene. For example, a reporter gene can encode a protein, for example, an enzyme whose activity can be quantitated, for example, chloramphenicol acetyltransferase (CAT) or firefly luciferase protein. Reporters also include fluorescent proteins, for example, green fluorescent protein (GFP) or any of the recombinant variants of GFP, including enhanced GFP (EGFP), blue fluorescent proteins (BFP and derivatives), cyan fluorescent protein (GFP and other derivatives), yellow fluorescent protein (YFP and other derivatives) and red fluorescent protein (RFP and other derivatives).

As used herein, the term “tag” as used in protein tags refers generally to peptide sequences that are genetically fused to other protein open reading frames, thereby producing recombinant fusion proteins. Ideally, the fused tag does not interfere with the native biological activity or function of the larger protein to which it is fused. Protein tags are used for a variety of purposes, for example but not limited to, tags to facilitate purification, detection or visualization of the fusion proteins. Some peptide tags are removable by chemical agents or by enzymatic means, such as by target-specific proteolysis (e.g., by TEV

Depending on use, the terms “marker,” “reporter” and “tag” may overlap in definition, where the same protein or polypeptide can be used as either a marker, a reporter or a tag in different applications. In some scenarios, a polypeptide may simultaneously function as a reporter and/or a tag and/or a marker, all in the same recombinant gene or protein.

As used herein, the term “prokaryote” refers to organisms belonging to the Kingdom Monera (also termed Procarya), generally distinguishable from eukaryotes by their unicellular organization, asexual reproduction by budding or fission, the lack of a membrane-bound nucleus or other membrane-bound organelles, a circular chromosome, the presence of operons, the absence of introns, message capping and poly-A mRNA, a distinguishing ribosomal structure and other biochemical characteristics. Prokaryotes include subkingdoms Eubacteria (“true bacteria”) and Archaea (sometimes termed “archaebacteria”).

As used herein, the terms “bacteria” or “bacterial” refer to prokaryotic Eubacteria, and are distinguishable from Archaea, based on a number of well-defined morphological and biochemical criteria.

As used herein, the term “eukaryote” refers to organisms (typically multicellular organisms) belonging to the Kingdom Eucarya, generally distinguishable from prokaryotes by the presence of a membrane-bound nucleus and other membrane-bound organelles, linear genetic material (i.e., linear chromosomes), the absence of operons, the presence of introns, message capping and poly-A mRNA, a distinguishing ribosomal structure and other biochemical characteristics.

As used herein, the terms “mammal” or “mammalian” refer to a group of eukaryotic organisms that are endothermic amniotes distinguishable from reptiles and birds by the possession of hair, three middle ear bones, mammary glands in females, a brain neocortex, and most giving birth to live young. The largest group of mammals, the placentals (Eutheria), have a placenta which feeds the offspring during pregnancy. The placentals include the orders Rodentia (including mice and rats) and primates (including humans).

A “subject” in the context of the present invention is preferably a mammal. The mammal can be a human, non-human primate, mouse, rat, dog, cat, horse, or cow, but are not limited to these examples.

As used herein, the term “encode” refers broadly to any process whereby the information in a polymeric macromolecule is used to direct the production of a second molecule that is different from the first. The second molecule may have a chemical structure that is different from the chemical nature of the first molecule.

For example, in some aspects, the term “encode” describes the process of semi-conservative DNA replication, where one strand of a double-stranded DNA molecule is used as a template to encode a newly synthesized complementary sister strand by a DNA-dependent DNA polymerase. In other aspects, a DNA molecule can encode an RNA molecule (e.g., by the process of transcription that uses a DNA-dependent RNA polymerase enzyme). Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

As used herein, the term “derived from” refers to a process whereby a first component (e.g., a first molecule), or information from that first component, is used to isolate, derive or make a different second component (e.g., a second molecule that is different from the first). For example, the mammalian codon-optimized Cas9 polynucleotides of the invention are derived from the wild type Cas9 protein amino acid sequence. Also, the variant mammalian codon-optimized Cas9 polynucleotides of the invention, including the Cas9 single mutant nickase and Cas9 double mutant mill-nuclease, are derived from the polynucleotide encoding the wild type mammalian codon-optimized Cas9 protein.

As used herein, the expression “variant” refers to a first composition (e.g., a first molecule), that is related to a second composition (e.g., a second molecule, also termed a “parent” molecule). The variant molecule can be derived from, isolated from, based on or homologous to the parent molecule. For example, the mutant forms of mammalian codon-optimized Cas9 (hspCas9), including the Cas9 single mutant nickase and the Cas9 double mutant null-nuclease, are variants of the mammalian codon-optimized wild type Cas9 (hspCas9). The term variant can be used to describe either polynucleotides or polypeptides.

As applied to polynucleotides, a variant molecule can have entire nucleotide sequence identity with the original parent molecule, or alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99% or more identical in nucleotide sequence compare to the original nucleotide sequence. Polynucleotide variants also include polynucleotides comprising the entire parent polynucleotide, and further comprising additional fused nucleotide sequences. Polynucleotide variants also includes polynucleotides that are portions or subsequences of the parent polynucleotide, for example, unique subsequences (e.g., as determined by standard sequence comparison and alignment techniques) of the polynucleotides disclosed herein are also encompassed by the invention.

In another aspect, polynucleotide variants includes nucleotide sequences that contain minor, trivial or inconsequential changes to the parent nucleotide sequence. For example, minor, trivial or inconsequential changes include changes to nucleotide sequence that (i) do not change the amino acid sequence of the corresponding polypeptide, (ii) occur outside the protein-coding open reading frame of a polynucleotide, (iii) result in deletions or insertions that may impact the corresponding amino acid sequence, but have little or no impact on the biological activity of the polypeptide, (iv) the nucleotide changes result in the substitution of an amino acid with a chemically similar amino acid. In the case where a polynucleotide does not encode for a protein (for example, a tRNA or a crRNA or a tracrRNA), variants of that polynucleotide can include nucleotide changes that do not result in loss of function of the polynucleotide. In another aspect, conservative variants of the disclosed nucleotide sequences that yield functionally identical nucleotide sequences are encompassed by the invention. One of skill will appreciate that many variants of the disclosed nucleotide sequences are encompassed by the invention.

Variant polypeptides are also disclosed. As applied to proteins, a variant polypeptide can have entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence.

Polypeptide variants include polypeptides comprising the entire parent polypeptide, and further comprising additional fused amino acid sequences. Polypeptide variants also includes polypeptides that are portions or subsequences of the parent polypeptide, for example, unique subsequences (e.g., as determined by standard sequence comparison and alignment techniques) of the polypeptides disclosed herein are also encompassed by the invention.

In another aspect, polypeptide variants includes polypeptides that contain minor, trivial or inconsequential changes to the parent amino acid sequence. For example, minor, trivial or inconsequential changes include amino acid changes (including substitutions, deletions and insertions) that have little or no impact on the biological activity of the polypeptide, and yield functionally identical polypeptides, including additions of non-functional peptide sequence. In other aspects, the variant polypeptides of the invention change the biological activity of the parent molecule, for example, mutant variants of the Cas9 polypeptide that have modified or lost nuclease activity. One of skill will appreciate that many variants of the disclosed polypeptides are encompassed by the invention.

In some aspects, polynucleotide or polypeptide variants of the invention can include variant molecules that alter, add or delete a small percentage of the nucleotide or amino acid positions, for example, typically less than about 10%, less than about 5%, less than 4%, less than 2% or less than 1%.

As used herein, the term “conservative substitutions” in a nucleotide or amino acid sequence refers to changes in the nucleotide sequence that either (i) do not result in any corresponding change in the amino acid sequence due to the redundancy of the triplet codon code, or (ii) result in a substitution of the original parent amino acid with an amino acid having a chemically similar structure. Conservative substitution tables providing functionally similar amino acids are well known in the art, where one amino acid residue is substituted for another amino acid residue having similar chemical properties (e.g., aromatic side chains or positively charged side chains), and therefore does not substantially change the functional properties of the resulting polypeptide molecule.

The following are groupings of natural amino acids that contain similar chemical properties, where substitutions within a group is a “conservative” amino acid substitution. This grouping indicated below is not rigid, as these natural amino acids can be placed in different grouping when different functional properties are considered. Amino acids having nonpolar and/or aliphatic side chains include: glycine, alanine, valine, leucine, isoleucine and proline. Amino acids having polar, uncharged side chains include: serine, threonine, cysteine, methionine, asparagine and glutamine. Amino acids having aromatic side chains include: phenylalanine, tyrosine and tryptophan. Amino acids having positively charged side chains include: lysine, arginine and histidine. Amino acids having negatively charged side chains include: aspartate and glutamate.

As used herein, the terms “identical” or “percent identity” in the context of two or more nucleic acids or polypeptides refer to two or more sequences or subsequences that are the same (“identical”) or have a specified percentage of amino acid residues or nucleotides that are identical (“percent identity”) when compared and aligned for maximum correspondence with a second molecule, as measured using a sequence comparison algorithm (e.g., by a BLAST alignment, or any other algorithm known to persons of skill), or alternatively, by visual inspection.

The phrase “substantially identical,” in the context of two nucleic acids or polypeptides refers to two or more sequences or subsequences that have at least about 60%, about 80%, about 90%, about 90-95%, about 95%, about 98%, about 99% or more nucleotide or amino acid residue identity, when compared and aligmed for maximum correspondence using a sequence comparison algorithm or by visual inspection. Such “substantially identical” sequences are typically considered to be “homologous,” without reference to actual ancestry. Preferably, the “substantial identity” between nucleotides exists over a region of the polynucleotide at least about 50 nucleotides in length, at least about 100 nucleotides in length, at least about 200 nucleotides in length, at least about 300 nucleotides in length, or at least about 500 nucleotides in length, most preferably over their entire length of the polynucleotide. Preferably, the “substantial identity” between polypeptides exists over a region of the polypeptide at least about 50 amino acid residues in length, more preferably over a region of at least about 100 amino acid residues, and most preferably, the sequences are substantially identical over their entire length.

The phrase “sequence similarity,” in the context of two polypeptides refers to the extent of relatedness between two or more sequences or subsequences. Such sequences will typically have some degree of amino acid sequence identity, and in addition, where there exists amino acid non-identity, there is some percentage of substitutions within groups of functionally related amino acids. For example, substitution (misalignment) of a serine with a threonine in a polypeptide is sequence similarity (but not identity).

As used herein, the term “homologous” refers to two or more amino acid sequences when they are derived, naturally or artificially, from a common ancestral protein or amino acid sequence. Similarly, nucleotide sequences are homologous when they are derived, naturally or artificially, from a common ancestral nucleic acid. Homology in proteins is generally inferred from amino acid sequence identity and sequence similarity between two or more proteins. The precise percentage of identity and/or similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95%, or 99% or more, can also be used to establish homology. Methods for determining sequence similarity percentages (e.g., BLASTP and BLASTN using default parameters) generally available.

As used herein, the terms “portion,” “subsequence,” “segment” or “fragment” or similar terms refer to any portion of a larger sequence (e.g., a nucleotide subsequence or an amino acid subsequence) that is smaller than the complete sequence from which it was derived. The minimum length of a subsequence is generally not limited, except that a minimum length may be useful in view of its intended function. The subsequence can be derived from any portion of the parent molecule. In some aspects, the portion or subsequence retains a critical feature or biological activity of the larger molecule, or corresponds to a particular functional domain of the parent molecule, for example, the DNA-binding domain, or the transcriptional activation domain. Portions of polynucleotides can be any length, for example, at least 5, 10, 15, 20, 25, 30, 40, 50, 75, 100, 150, 200, 300 or 500 or more nucleotides in length.

As used herein, the term “kit” is used in reference to a combination of articles that facilitate a process, method, assay, analysis or manipulation of a sample. Kits can contain written instructions describing how to use the kit (e.g., instructions describing the methods of the present invention), chemical reagents or enzymes required for the method, primers and probes, as well as any other components.

### EXAMPLES

### Example 1

### General Methods

Cas 9-Associated Genes and Bacterial Strain

Bacterial Streptococcus pyogenes cas9 gene with deactivated nuclease activity was obtained from Addgene (ID: 48657). S.pyogenes sgRNA was obtained from Addene (ED: 44251). Escherichia coli K-12 ER2267 obtained from New England Biolabs (NEB) has the following genotype: F′ proA⁺B⁺ lacI^{q} Δ(lacZ)M15 zzf::mini-Tn10 (Kan^{R})/Δ(argF-lacZ)U169 glnV44 el4⁻(McrA⁻) rfbD1? recA1 relA1? endA1 spoT1? thi-1 Δ(mcrC-mrr)114::IS10.

General Methods and Reagents for Plasmid Construction

General enzyme reagents for plasmid or gene construction include Quick ligation kit (NEB), Phusion Master Mix (NEB), Gibson Assembly Master Mix (NEB) and GoTaq DNA polymerase (Promega).

Site 1 with varying gap length was added onto pdimn2 plasmid. Short double stranded DNA containing variations of site 1 was created using primers from IDT and Phusion Master Mix. The double stranded oligonucleotide was joined to the linearized pdimn2 vector using Gibson Assembly Master Mix (GAMM) at insert to vector ratio of 5:1 and total DNA mass of 50-100 ng in a volume of 10.4 μL. Gibson assembly ligation mixture was transformed into chemically competent ER2267 cells (100 μL). Transformation was recovered at 37C for 1 hour and plated on Ampicillin (100ug/mL) and 2% wly glucose supplemented Luria Broth plates.

Plasmid Modifications

DNA sequence for sgRNA1 was inserted in the pARC8 plasmid, along with J23100 promoter and terminators upstream and downstream of the sgRNA sequence. Four FspI sites from S. Pyog dCas9 gene were removed by silent mutations.

In Vivo Methylation

Culture of ER2267 was started in 5 mL, Luria Broth supplemented with glucose (0.2% w/v), Ampicillin (1100 μglmL) and Chloramphenicol (50 μg/mL). Arabinose (0.0167% w/v) was added to induce expression under pBad promoter, and 1 mM IPTG for Lac promoter. Cultures were incubated overnight at 37 C and shaken at 250 RPM. After, they were pelleted at 3000 RPM for 5 minutes and plasmids were extracted with QIArep Spin Miniprep Kit (Qiagen).

Restriction Digestion Assay and DNA Electrophoresis

Plasmid. DNA (160-180 ng) was digested for at 37° C. for 1.5 hour with SacI-HF (10 units) and FspI (2.5units) in 1× Cutsmart buffer in 10-μL reaction volume. Enzymes and reaction buffer were obtained from NEB. DNA reaction was loaded into 1.5% w/v TAE gel and electrophesed at 110 Volts for 50 minutes. Band patterns were visualized under UV lighting and imaged with Gel Logic 112 from Carestream.

Bisulfite Sequencing Assays in Mammalian Cells

Plasmids containing the dCas9-M.SssI constructs can be transformed into any cell line for analysis. Currently all experiments have been done using the HEK293T cell line but cell lines can be changed depending on methylation status of specific promoters. Cells are seeded at 5×10⁵ cells per well and allowed to grow overnight to approximately 50% confluence before transfection. Plasmids were transfected using Lipofectamine 2000 or Optifect (Invitrogen) using manufacturer's recommendations. Transfection reagent and media is removed after 24 hours and replaced with fresh media. Cells are recovered at 48 hours after transfection and sorted using the Sony SH800 flow cytometer (Dana-Farber Cancer Institute Flow Cytometry Core Facility) based on GFP fluorescence. GFP positive cells were then lysed and underwent bisulfite conversion using the Epitect Fast DNA Bisulfite Kit (Qiagen). Converted DNA was then amplified using primers designed for the converted HBG1 locus and containing a KpnI and SphI sites for cloning (Primers:

BisHBG1-for-
5′-CTCCGTAGGTACCGTTAAAGGGAAGAATAAATTAGAGAAAAATTGG,
and
BISHBG1endog-rev-
5′-TCAGTGCATGCCTTACCCCACAAACTTATAATAATAACC).

Sample PCR was then digested with 20U of KpnI-HF and SphI-HF (New England Biolabs) and ligated into a pUC19 vector. Ligations were transformed into New England Biolab's NEB Turbo cells (F′ proA⁺B⁺lacI^{q}ΔlazZM15/fhuA2 Δ(lac-proAB) glnV galK16 galE15 R(zgb-210::Tn10)Tet^{S} endA1 thi-1 Δ(hsdS-mcrB)5) and plated on LB-Amp plates. Colonies (10-20) were then picked the next day and sequenced by outside vendor (Genewiz).

### Example 2

### Demonstration of Targeted Methylation with an Artificially Bisected M.SssI

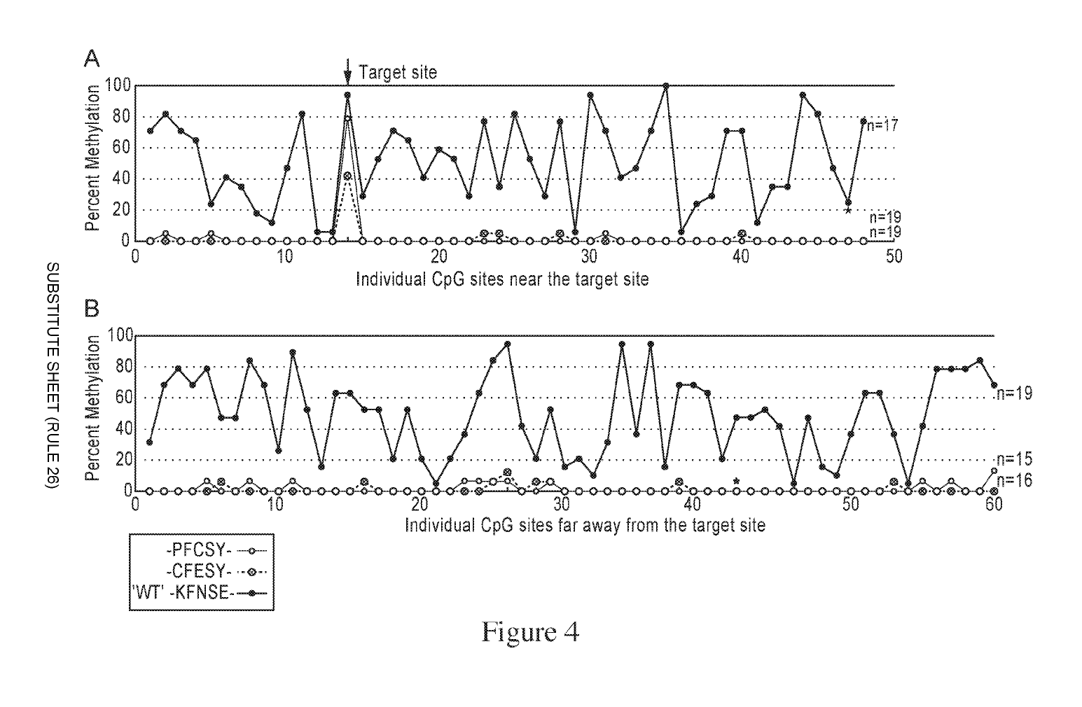

The bacterial M.SssI MTasel 6 recognizes the sequence 5′-CG-3′ (i.e. CpG) and methylates the cytosine. Compared with M.HhaI, M.SssI is a more useful bacterial MTase to convert into a targeted MTase, since theoretically it could be engineered to methylate any CpG site. A crystal structure of M.SssI does not exist, so we used a homology model based on the M.HhaI structure and sequence aligninents 46 to predict an equivalent bisection site in M.SssI. We made an analogous construct to the best performing M.HhaI construct described above. Although the bifurcated M.SssI construct methylated the target site, it also methylated other M.SssI sites15. We sought to reduce off-target methylation without affecting levels of methylation at the target site. We developed a directed evolution strategy (see FIG. 7) to improve the targeting of MTases toward new sites and used this strategy to optimize our M.SssI fusion construct9. We constructed a library in which a region of the C-terminal fragment of the M.SssI protein that makes non-specific contact with the DNA (i.e. a region that interacts with the DNA backbone, not the bases) was randomized by cassette mutagenesis. We performed a negative selection against off-target methylation and a positive selection for methylation at a target site in vitro. This strategy allowed us to quickly identify variants with improved targeting ability and activity in vivo. The unprecedented high specificity of two of the constructs was demonstrated by bisulfite sequencing, which indicate at least a 100-fold preference for methylating the on-target site over the off-target site (i.e. variant PFCSY caused 80% methylation at the target site and 0.8% methylation at all other sites) (FIG. 4). The methylation specificity may be >100-fold because low level incomplete conversion during bisulfate sequencing commonly occurs, which would manifest as a low level of apparent methylation at the non-target sites. This work was featured in an article on targeting DNA methylation to the genome in the September 2014 issue in Biotechniques 47. However, the drawback of the M.SssI-ZF split MTases is that the zinc finger must be redesigned for each new target, and such redesign is not a trivial task. Thus, we have proceeded with developing a split M.SssI using dCas9 to target the methylation instead of zinc fingers.

### Example 3

### Demonstration of Biases Methylation Using Split M.SssI Fused to dCas9

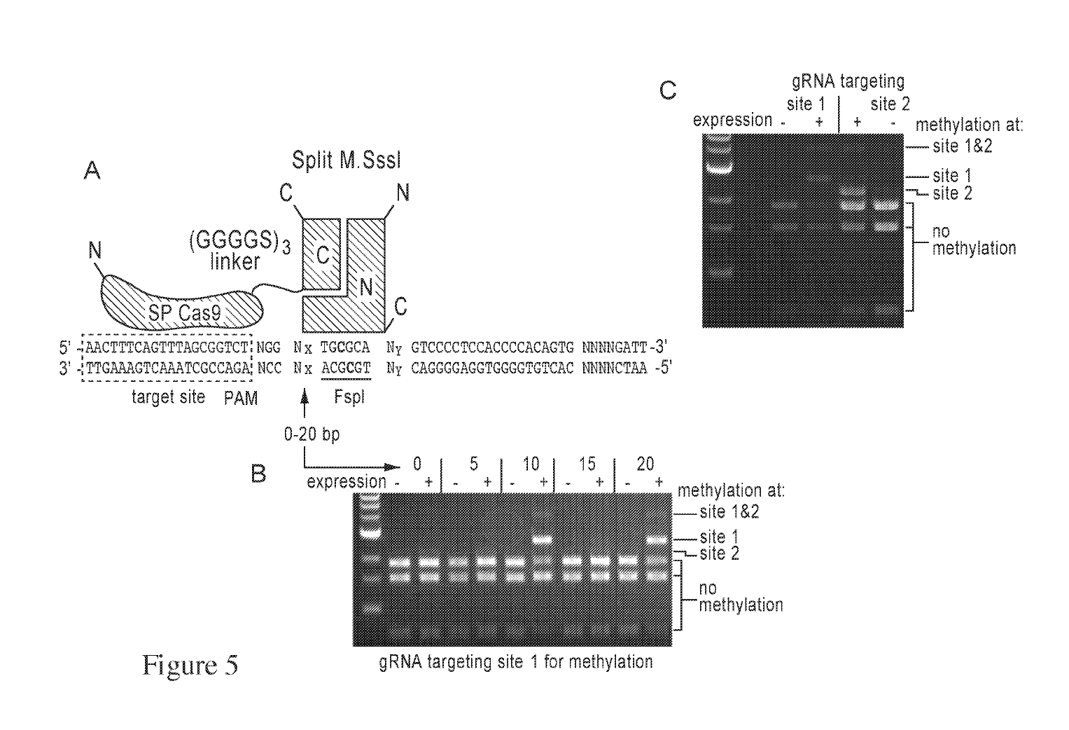

As an initial test of the capacity of dCas9 to provide modular, targeted methylation, we fused the C-terminal fragment of the split M.SssI to the dCas9 from Streptococcus pyogenes (FIG. 5A). This construct, despite having only one half fused to a DNA binding protein, provided a surprising degree of bias towards the desired target site 1 (as defined by the co-expressed gRNA), provided the protospacer site for dCas9 binding was an appropriate distance (the “gap” DNA) from the site to be methylated (FIG. 5B). In follow-up experiments (not shown) in which the gap DNA was varied by every 2 by up to 20 bp, biased methylation occurred at gap DNAs of length, 6, 8, 10, 12, 18 and 20. This periodicity makes sense based on the periodicity of DNA (i.e. one turn of the double helix is 11 bp). We next demonstrated modularity by designing a gRNA to guide methylation to site 2 instead of site 1. The methylation bias inverted as desired towards site 2 (FIG. 5C). This result is highly significant. Without altering the protein in FIG. 5A, we could direct the protein to methylate a new site just by changing the gRNA using simple base-pairing rules. Furthermore, unlike site 1, for which we used a well-characterized gRNA demonstrated to work with the Cas9 protein, the DNA flanking site 2 was not designed at all. This DNA sequence was just the DNA that happened to be near an FspI site in the plasmid serving as our negative control. We searched for a suitable PAM site nearby (one was available with a DNA gap of 9 bp) and designed the gRNA accordingly. This is essentially what would have to be done for research and therapeutic applications.

We anticipate improvements in targeting by introducing those mutations in the C-terminal fragment and fusing the N-terminal fragment of M.SssI to a separate dCas9.

### Example 4

### Create Modular, Targeted Cytosine MTases Capable of Achieving>95% Methylation at a Desired Target Site with Undetectable Methylation at Non-Target CpG Sites

We will reengineer M.SssI to be capable of specifically methylating a select target CpG site and not other CpG sites (M.SssI normally methylates all CpG sites). Non-target methylation will be prevented by splitting M.SssI into two fragments that do not appreciably assemble into an active enzyme in unassisted fashion. Instead, methylation will be directed to target a particular CpG site by orthogonal dCas9s fused to each of the M.SssI fragments. The target CpG sites will be defined by flanking sequences to which the dCas9 domains bind, as directed by the gRNA that are coexpressed. We have preliminary evidence that this strategy can bias M.SssI activity towards a target site (FIG. 5). The goal of this aim is to improve the specificity and activity such that the engineered enzymes are capable of >95% methylation at the target site with minimal (<1%) methylation at non-target sites. This optimization will be guided by our previous experience in designing targeted MTases fused to zinc fingers 9, 14, 15 and will use a number of strategies and assays developed in the Ostermeier lab.

### Example 5

### Optimization of the dCas9-M.SssI Split MTase

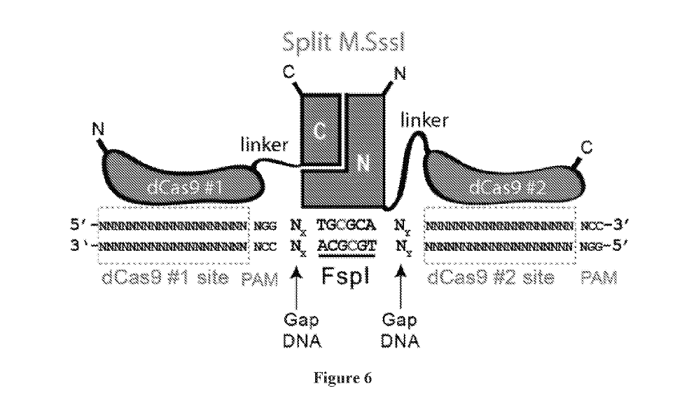

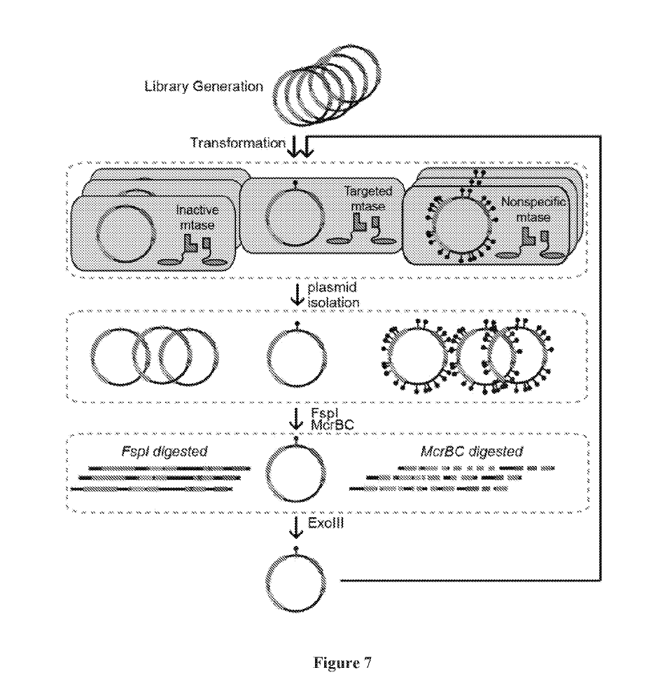

A general schematic of the dCas9-M.SssI split MTase is shown in FIG. 6. The MTase fragments will be fused to orthogonal dCas9, the Streptococcus pyogenes dCas9 used in our preliminary data and dCas9 from Neisseria meningitidis. Orthogonal dCas9s are preferred so that the correct pairs of MTase fragments assemble at the target site in the correct orientation. Orthogonality is determined by the need for different PAM sites and different gRNA sequences (i.e. differences apart from the spacer sequence). Parameters to consider during optimization include the length and composition of the peptide linkers between dCas9 and the MTase fragments and the length of the gap DNA between the site to be methylated and the dCas9 binding site. Although not shown in FIG. 6, the linear order of the fusions (i.e. is the dCas9 fused to the N- or the C-terminus of the MTase fragment) and the relative orientation of the dCas9 binding sites (i.e. whether dCas9 binds to the top or bottom strand) are also design considerations. However, FIG. 6 shows our expectation for the most useful geometry based on our ZF-M.SssI fusions i.e. that fusion of each dCas9 to the site of bisection of the enzyme will be most useful). We have already shown that fusion of the C-terminal fragment in this geometry results in biased methylation towards the target site (FIG. 5).

As in our previous work using zinc fingers our optimization will proceed using at iterative process, which will be aided by the crystal structure of S. pyogenes Cas948. Parameters such as peptide linker and gap DNA length will be systematically varied and tested using our simple restriction enzyme protection assay (FIG. 2). In this assay we use E. coli strain ER2267 (New England BioLabs), which harbors genomic modifications making it tolerant to CpG methylation. To maximize the mixing and matching of fragments, the two fragments will be encoded on separate compatible plasmids and will be under separate inducible promoters (tac and PBAD), with one plasmid also containing the target site for methylation and a control non-target site, much like in some of our previous work Through this optimization, we will also learn of the range of gap DNA for which targeted methylation occurs. This information is very important for future targeting of methylation of a genome, because one must locate two suitable PAM sequences nearby the desired site to be methylated. Knowing the flexibility in the length of the gap DNA will make it more likely that a suitable site for designing the gRNA can be identified.

We will define the fusion geometry, linker length, and gap DNA lengths that are compatible with biased methylation to a desired target site.

### Example 6

### Experimental Optimization by Directed Evolution

Our experience engineering M.HbaI-ZF and M.SssI-ZF targeted MTases tells us that, through optimization, we will be able to improve our engineered split M.SssI variants to have a strong bias for methylation at a desired target site. However, we have yet been able to engineer an MTase with >95% methylation at the target site without also observing some methylation at non-target sites at high expression levels.

We will first introduce mutations improving specificity identified in our previous study, but we have plans for achieving desirable further improvements. Further improvements in targeted MTase activity and specificity will be achieved through mutagenesis coupled with a unique selection strategy for efficient targeted methylation. The following mutagenesis strategies will be pursued in parallel: (1) site-specific, site-saturation mutagenesis at the bisected M.SssI interface designed to reduce the affinity that the two fragments have for each other and (2) site-specific, site-saturation mutagenesis to reduce the affinity of the M.SssI domain for DNA (i.e. the mutations that increase the Km through decreased affinity but do not effect kcat appreciably). The later strategy we successfully employed with ZF-M.SssI MTases9 (FIG. 4).

The sites for mutagenesis fix (1) and (2) will be chosen based on previous studies 49, 50 and our homology model of M.SssI. We expect that modulation of the M.SssI variants' intrinsic activity (by mutation) and expression level may be necessary, because reductions in M.SssI fragment's association with each other and with DNA may require compensatory increases in cellular enzyme activity. For (1) and (2) we will carry out site-saturation mutagenesis at multiple sites simultaneously using our recently developed PFunkel mutagenesis technique. PFunkel mutagenesis makes a number of improvements on classic Kunkel mutagenesis. The method allows one to create libraries in which up to four or more positions scattered across the protein can mutagenized at nearly 100% efficiency in a single round of mutagenesis.

All mutagenesis libraries will be subjected to a selection strategy for a targeted MTase that removes all plasmids not methylated at the target site and all plasmids that are methylated at more than one site (FIG. 7). The latter step makes use of the unusual endonuclease McrBC, which requires CpG methylation at two half sites located at different locations on the plasmid. We have used this process successfully on our ZF-M.SssI MTases9 resulting in improvements in targeting the MTase to the desired site (FIG. 4). Multiple rounds of selection can be used to achieve the enrichment necessary to find rare library members. The methylation specificity of the selected library members will be confirmed by resistance to FspI/McrBC double digestion, quantified by an FspI digestion assay, and confirmed by bisulfite sequencing. Beneficial mutations from both libraries will be combined and tested. Modularity will be confirmed by changing gRNA sequences as in FIG. 5C. Specificity will also be examined on the E. coli chromosome, which has five million bp and therefore contains about three orders of magnitude more off-target CpG sites than our plasmid DNA. We will use DNA immunoprecipitation (against methylated CpG sites) to quantify the extent of off-target methylation on the E. coli chromosome56. For comparison, we will examine cells expressing wildtype M.SssI and cells lacking the ability to methylate cytosine.

We will create modular MTases capable of methylating a target site at >95% efficiency while leaving non-target sites unmethylated (<1% methylation).

### Example 7

### Develop an Experimental System for Assessing and Defining dCas9-MTase/gRNA Specificity

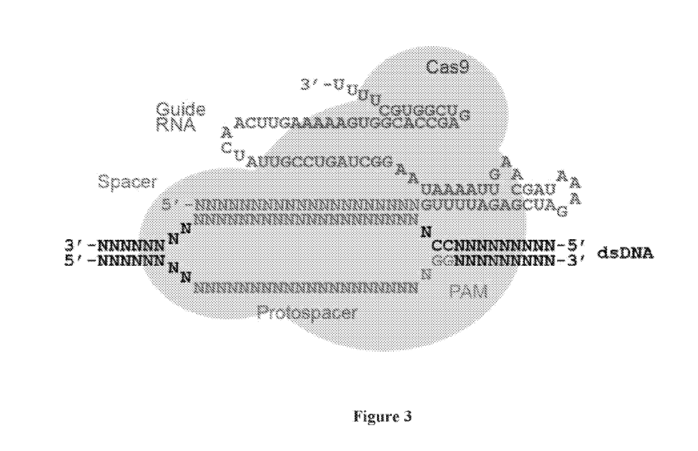

The specificity of our engineered enzymes for the target site will be further addressed by developing a reverse selection method for experimentally assessing and defining dCas9-MTase/gRNA specificity. En other words, we will develop a system for defining the protospacer determinants for dCas9-gRNA binding in the context of our MTase. Although the protospacer sequence (i.e. the DNA binding site of the gRNA; see FIG. 3) is 20 by in length, very recent studies suggest that dCas9 specificity is dominated by the 5-10 bp nearest the PAM site. We will develop a reverse selection method (i.e. identify from a library of protospacer sites the sequences at which a dCas9-MTase binds and effectively methylates). Since a library in which all 20 by of the protospacer are varied cannot be comprehensively evaluated, we will construct two N10 libraries in which the variability will be located either nearest the PAM site or furthest away. From these libraries, any protospacer sequence that directs the MTase to methylate the target CpG site can be identified using an in vitro selection for protection from FspI digestion. Plasmid DNA recovered will be subjected to deep sequencing, to characterize the protospacer binding specificity. Note that because our dCas9-MTases will require binding of two dCas9 domains at sites flanking the target site for methylation, each dCas9 need not have 20 bp specificity for our MTases to effectively target specific sites in the genome. Each dCas9 may need only 8 bp or less of specificity, as a random sequence of 16 bp occurs once every 416=˜4.2 billion bp and the human genome is 3.2 billion by in length. Additionally, a significant fraction of the human genome is likely inaccessible due to chromatin inaccessibility.

We will develop a reverse selection system for assessing dCas9-MTase/gRNA specificity, which will further define the MTase specificity and will be useful in designing gRNA.

### Example 8

### Evaluating the Effect of DNA Gap on Methylation

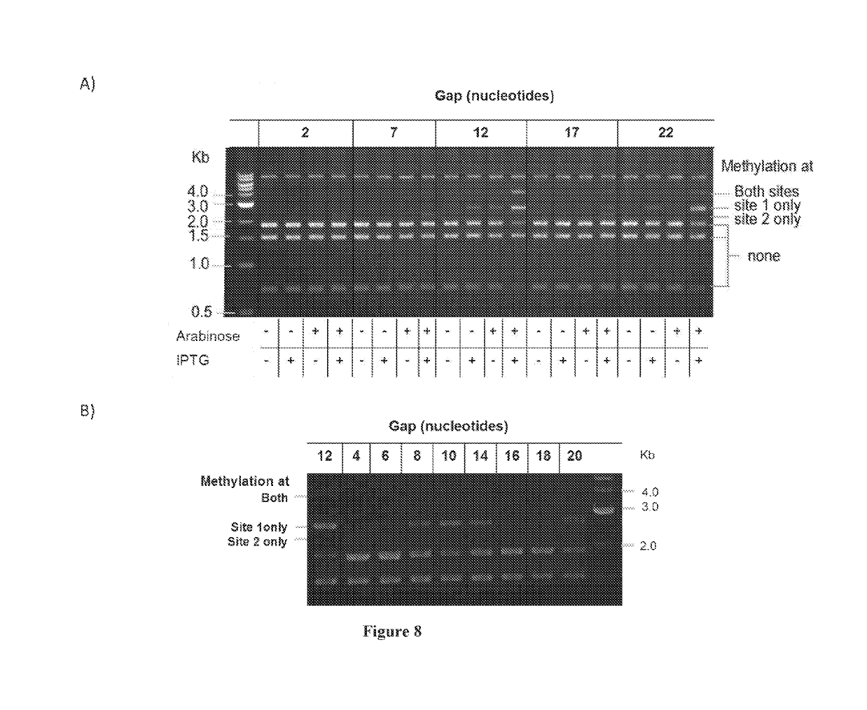

We further verified the effect of the DNA gap on methylation by expressing both fragments with gap lengths 4, 6, 8, 10, 14, 16, 18, and comparing methylation with gap length 12 (FIG. 8B) . Methylation at only the target site is absent for gap 4 and 6, and 16 and 18. Interestingly, gap length 6 and 8 are expected to have no methylation at the target site since gap length 7 has less methylation at target than off-target site (FIGS. 5B and 8B). We think a C-terminal fusion of Cas9 with M.SssI impedes targeted methylation when gap is with 6 nt.

We confirm methylation without both fragments results in little to no methylation. When only one of two fragments is induced low methylation is levels of methylation is observed (FIG. 8a). We believe this is due to low levels of leaky expression from lac promoter and pBAD. Still, the result points to the synergistic effect on methylation from the assembly of both fragments.

### Example 9

### SgRNA: Crucial for M.SssI Targeting

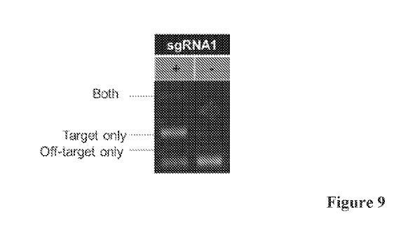

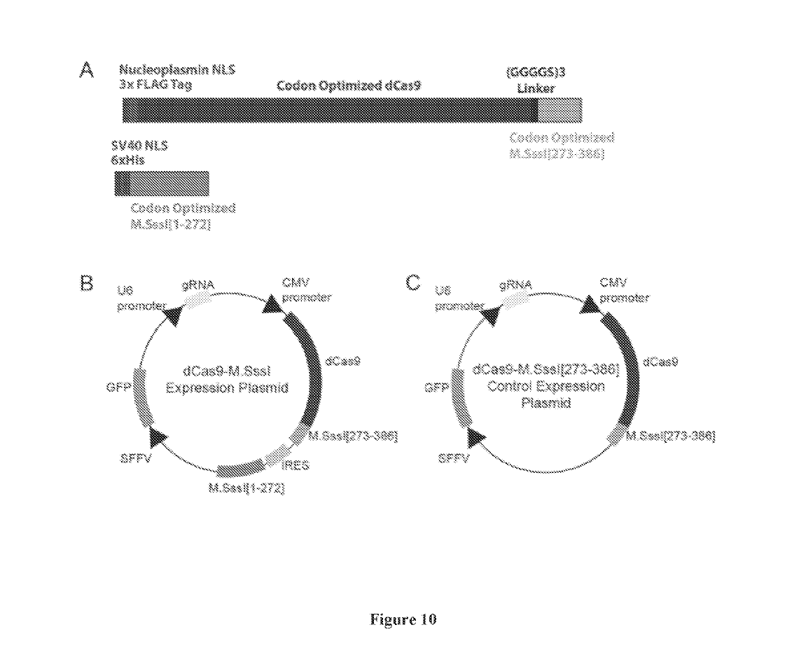

Assembly of M.SssI fragments without dCas9 binding may be possible because of the flexibility imparted on the linkers that join the dCas9-(GGGGS)₃-M.SssI[273-386]. We test this by expressing both methyltransferase fragments in the presence and absence of the sgRNA1 (FIG. 9). With sgRNA, methylation at both sites and at the target site only is increased. However, increase in methylation at the target site is significantly higher. A low and almost undetectable amount of methylation is observed when sgRNA is removed.

### Example 10

### Use of dCas9-M.SssI Constructs in Mammalian Cells

All dCas9-M.SssI constructs have to be modified and re-optimized for use in eukaryotic cells. Many parameters determined for active constructs in E. coli such as linker length, DNA gap lengths and spatial orientation will be similar and translate to use other organisms. However, the increased complexity of eukaryotic cells; including the sequestration of the chromatin in the nucleus, effect of chromatin structure on DNA accessibility, and increased size of the cell present additional challenges to targeted DNA methylation. As the specificity of the split-M.SssI fusions are sensitive to concentration in the cell, expression levels have to be optimized for each new system.

Several modifications were made to allow for expression and nuclear localization in mammalian systems. The coding sequences for the S. pyog dCas9 and M.SssI fragments were codon optimized for expression in human cells. Nuclear localization signals (NLS) were added to constructs to allow for trafficking of proteins into the nucleus and tags (Flag and 6× His) were added for use in western blots or localization studies. Additionally new expression vectors were created for use in mammalian cells consisting of the dCas9-M.SssI fragments under different mammalian promoters, the sgRNA under control of the U6 promoter, a fluorescent marker (eGFP) to allow for sorting of cells containing plasmid, as well as an antibiotic resistance gene and bacterial origin for cloning purposes (FIG. 10).

### Example 11

### Demonstration of Targeted Methylation in the HBG1 Promoter Region

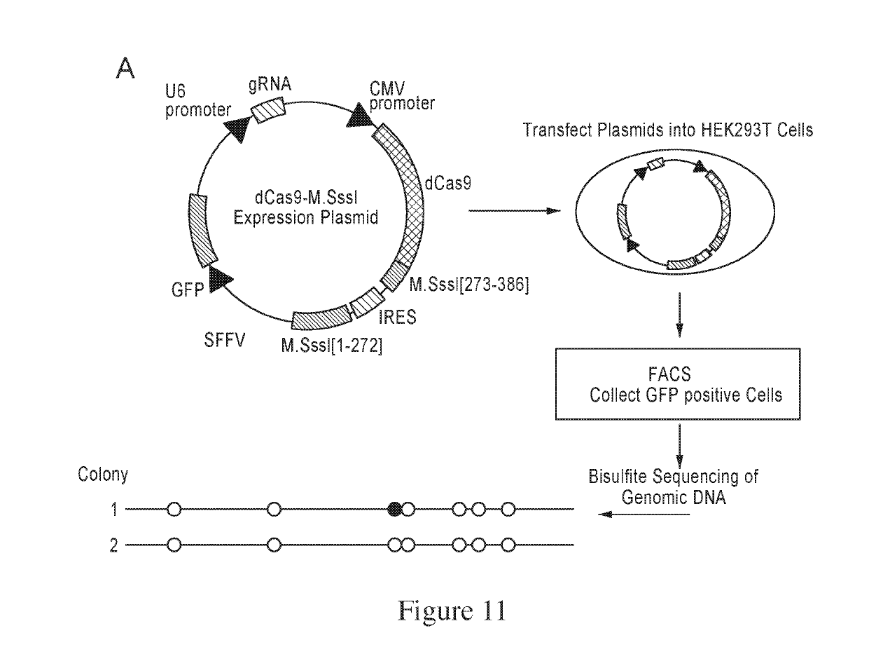

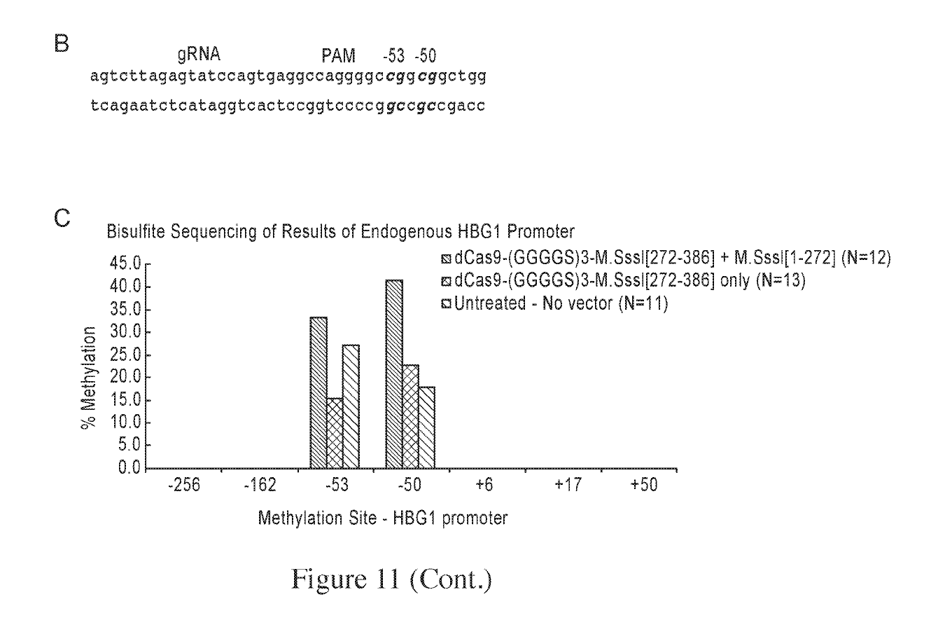

As proof of concept we attempted to target the dCas9-(GGGGS)₃-M.SssI [273-386] and the untethered M.SssI [1-272] constructs to the HBG1 promoter in HEK293T (Human Embryonic Kidney) cells. HBG1 is a gene that codes for the fetal-hemoglobin protein in humans. The promoter contains 7 CpG sites and a PAM sequence was found to be located 8 and 11 bp upstream of 2 CpG sites (FIG. 11B). These sites should be targetable based on previous analysis of the gap DNA requirements with these constructs. We created a sgRNA targeted to that site and inserted it into our expression vectors. We transfected both expression vectors into HEK293T cells and isolated genomic DNA from GFP positive cells (FIG. 11A and Methods section). Bisulfite sequencing of the extracted DNA showed a preferential increase in methylation at the −53 site (42%) compared to untreated cells (18.2%) (FIG. 11C) There was not a significant increase in the −50 site perhaps due to it being too close to the PAM site as seen in E. coil studies.

### Example 12

### Dual-Fluorescent Reporter Plasmid for Identification of Functionally-Repressive CpGs And Site-Specific gRNAs

Our goal is development of a user-friendly reporter plasmid for rapidly screening gRNAs and identifying repressive sites in mammalian promoters. Our reporter vector will be CpG-free backbone engineered with multiple cloning sites for rapid and directional insertion of test promoter fragments upstream of red fluorescent protein (mCherry). A methylation-resistant control promoter is cloned upstream of blue fluorescent protein (BFP) to allow for normalization of mCherry expression. By utilizing a reporter plasmid we ensure that (1) the promoter is 100% unmethylated initially, (2) the promoter is not blocked by higher chromatin structures and is accessible to our dCas9-MTase fusions, and (3) gene expression is easily quantifiable by flow cytometry analysis. Preliminary experiments show that a test promoter containing a CpG island shows over a 90% decrease in mCherry expression when fully methylated in vitro with a CpG MTase in comparison to an unmethylated plasmid. Both methylated and unmethylated plasmids show similar levels of BFP expression. Additionally, plasmids maintain the original methylation status even after being in cells for 48 hours.

We will order small combinatorial libraries of chemically-synthesized gRNAs arrayed in 96 well format (Integrated DNA Technologies). There are several programs, such as CasFinder60, that can analyze DNA for potential gRNA target sites and evaluate potential off-target binding sites in the genome. While regions of DNA can have several potential PAM sites, gRNA pairs for a given targeted will be limited based on the permissible spacing of Cas9 target sequences from CpG sites.

As a first test target we will attempt to silence the hypoxia inducible factor 1α (HIF-1 α) gene. HIF-1α is upregulated in many solid tumors and is associated with poor prognosis of cancer patients61. It has been shown that a ˜130 bp region containing 14 CpG sites is demethylated resulting in increased expression. This will allow us to limit our initial gRNA library size by focusing on a small region of a CpG island that has been shown to be clinically relevant.

Reporters will be arrayed into 96 well plates with gRNAs and transfected with Lipofectamine2000 reagent (Life Technologies). Each well will have 10-20 gRNAs (5-10 gRNA pairs for the two dCas9-M.SssI fragments). We will then perform reverse transfection of a Cas9-M.SssI-expressing cell line or a demethylase plasmid. After 48 hours, we will perform FACS analysis to assess the degree of reduced expression of mCherry DNA will be extracted from cells expressing reduced mCherry, will be bisulfite treated, and promoter amplicons will be pyrosequenced to evaluate the percentage methylation at each CpG site.

### Example 13

### Validate Site-Specific CpG Methylation at Endogenous Loci

The preceding studies will identify the CpGs whose methylation led to decreased mCherry expression and the gRNAs that direct dCas9-M.SssI fusion partners to relevant sites using a reporter assay. However, these studies will not determine whether the comparable segments of the endogenous promoters (i.e. promoters on the chromosome and not on reporter plasmid) are equally accessible or whether the methylation of the endogenous site will be stably repressed over time and to the same extent as that same site in the context of our reporter assay. We will therefore test individuals and pools of gRNAs leading to reduced mCherry expression in the reporter assays above at endogenous promoters.

To determine whether a particular gene is expressed, we will perform RT-qPCR and Western blotting to quantify expression of the endogenous gene in multiple transfectable cell lines. We will use cancer cell lines as our starting point for several reasons. Cancers are generally characterized by global hypomethylation 65. Although, there are often areas of focal methylation (near tumor suppressor genes in a process called epimutation, not all tumors demonstrate focal methylation. Global hypomethylation in cancers provides us with the maximal opportunity to find unmethylated endogenous promoters in transfectable cell lines. Moreover, as an. Associate Member of Broad Institute, the Novina lab has access to the Cancer Cell Line Encyclopedia (CCLE), a library of more than 1000 cell lines representing virtually all cancers. These cancer cell lines have been globally annotated by genetic amplifications, deletions, mRNA and microRNA expression and, in limited cases, by methylation status. We will therefore choose representative cell lines where test promoters are expressed. We will validate this data by performing RT-qPCR to verify expression levels and will also perform bisulfite sequencing of the entire endogenous promoter in those cell lines demonstrating robust expression of the test gene.

We will transfect inducible dCas9-MTase expression constructs in selected cell lines and sort for GFP expressing cells. We will next transfect gRNAs and add tetracycline for 24-48 hours. We assess Cas9-M.SssI expression at 24 and 48 hours and will attempt to match dCas9-MTase levels that led to site-directed methylation in our reporter assays. We will remove tetracycline and allow the Cas9-MSssI levels to drop down to pre-induction levels and then will examine DNA methylation efficiency by bisulfite sequencing and target gene repression by RT-qPCR.

For gRNAs leading to target gene methylation and repression we will also examine off-target and unintended effects of dCas9-MTase expression using Illumina whole-genome bisulfite sequencing and RNA-seq. DNA methylation and gene induction will also assessed at later time points (>1 week in culture). This will also give us a preliminary assessment of the duration and heritability of repressive marks left on endogenous promoters.

These data will provide (1) high-resolution maps of the methylation status of the endogenous promoters in chosen cell lines, (2) a solid baseline for comparison of changes in methylation status after transduction of our dCas9-MTase-expressing constructs and (3) will thereby allow us to determine whether the observed methylation is a result of the engineered fusions' activity. We will identify the key sites of repressive methylation in test promoters and gRNAs that mediate efficient gene silencing. We will confirm the efficiency and stability of repressive marks at the endogenous promoters.

### Example 14

### Optimization of the dCas9-M.Sssl[273-386] Free M.Sssl[1.-2721 Split Methyltransferase System for Expression in Mammalian Cells

Optimization Variables

Nuclear Protein Levels

Expression levels and localization in mammalian cells can have an effect on the bifurcated M.SssI methyltransferase variants. Both fragments of the M.SssI must be expressed in high enough amounts and be present in the nucleus in order for them to reassemble at a target site on the genomic DNA. Protein levels in the cell can be adjusted by both vector design (promoter strength, vector size, and use of IRES vs separate promoters for fragments) as well as codon optimization to adjust translation speed and efficiency. Additionally folded proteins must then be trafficked to the nucleus in high enough amounts in order for them to methylate genomic DNA. Nuclear localization is usually accomplished through the addition of nuclear localization signals—amino acid sequences that allow for the protein to be imported into the nucleus. For larger proteins it is not uncommon for multiple NLS to be present to increase nuclear localization. Placement and number of the NLS can alter the efficiency of proteins to be trafficked the nucleus.

dCas9-M.Sssl Linker Design

Linker length and composition between the M.SssI fragments and its DNA binding domains can also effect methylation efficiency and the number and locations of sites that can be methylated with a given construct. Linkers that are too short may not be able to reach to target sites further away from a dCas9 binding site or wrap around the DNA to allow for proper orientation for M.SssI DNA binding. Composition of amino acids will also affect the range of spatial orientations the methyltransferase and DNA binding domains can have depending on the preferred structure flexibility of the amino acid sequence. Initial constructs used a very flexible (GGGGS)3 linker composed mostly of the small non-polar amino acid residue glycine connecting the M.SssI fragment to a catalytically dead S. pyogenes Cas9 (dSPCas9). However, potential binding sites of the dSPCas9 are limited by the necessity of having a compatible PAM binding site for S. pyogenes. Therefore having a longer linker capable of allowing the attached M.SssI fragment to reach multiple CpG sites around a single dCas9 binding site is advantageous.

Testing Different Codon Optimization, Linker and Nuclear Localization Variants of dCas9-M.SssI[273-386] and M.SssI[1-272] for Methylation Activity in Mammalian Cells

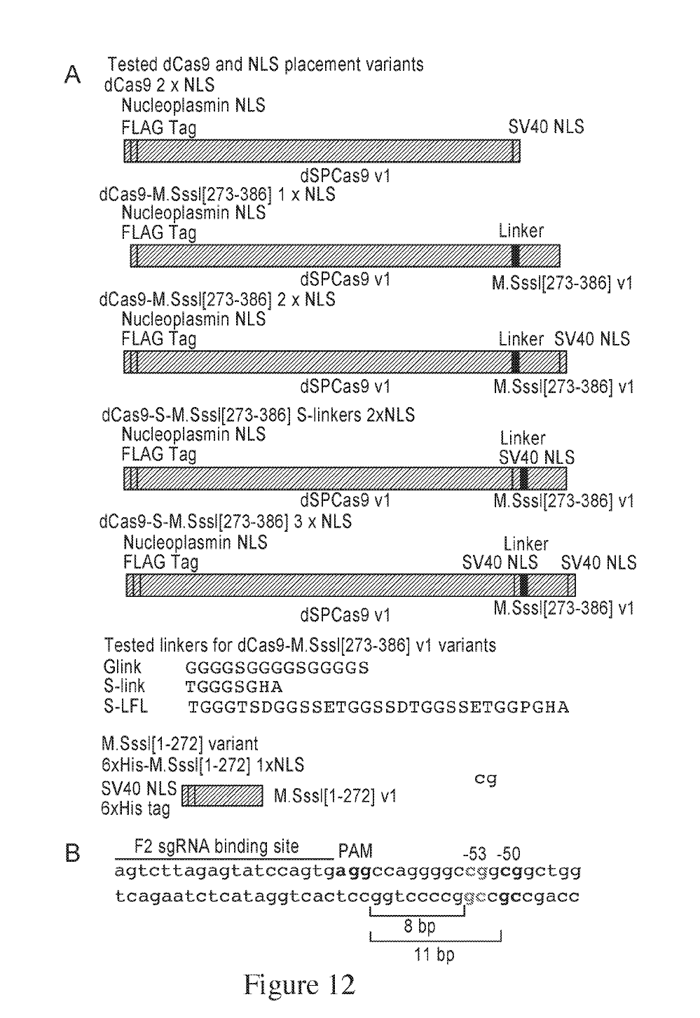

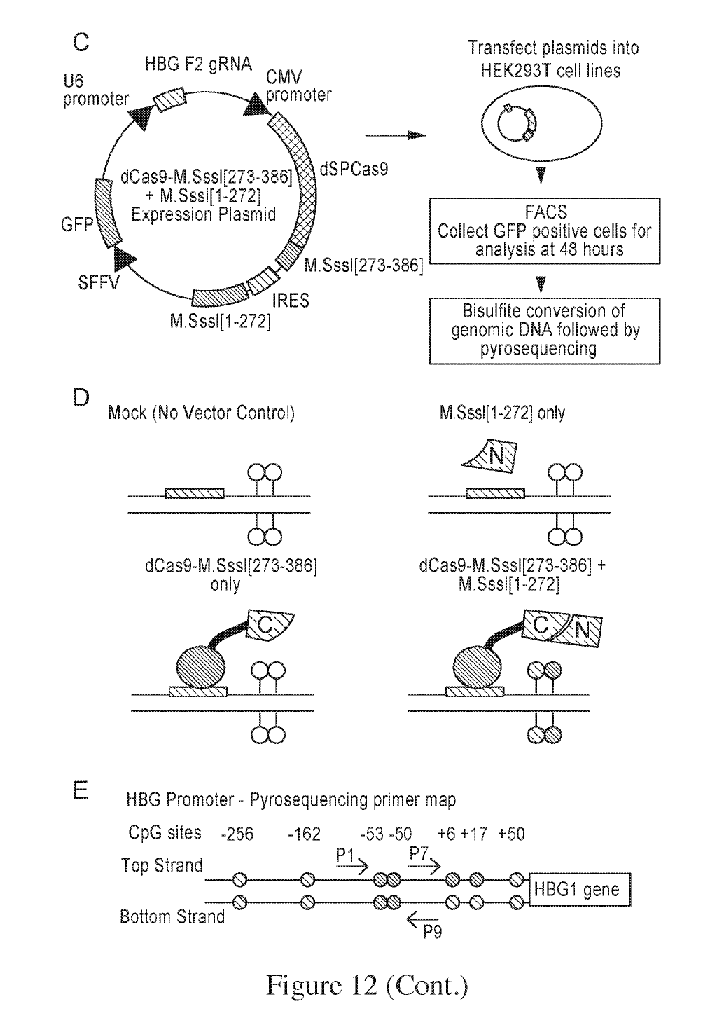

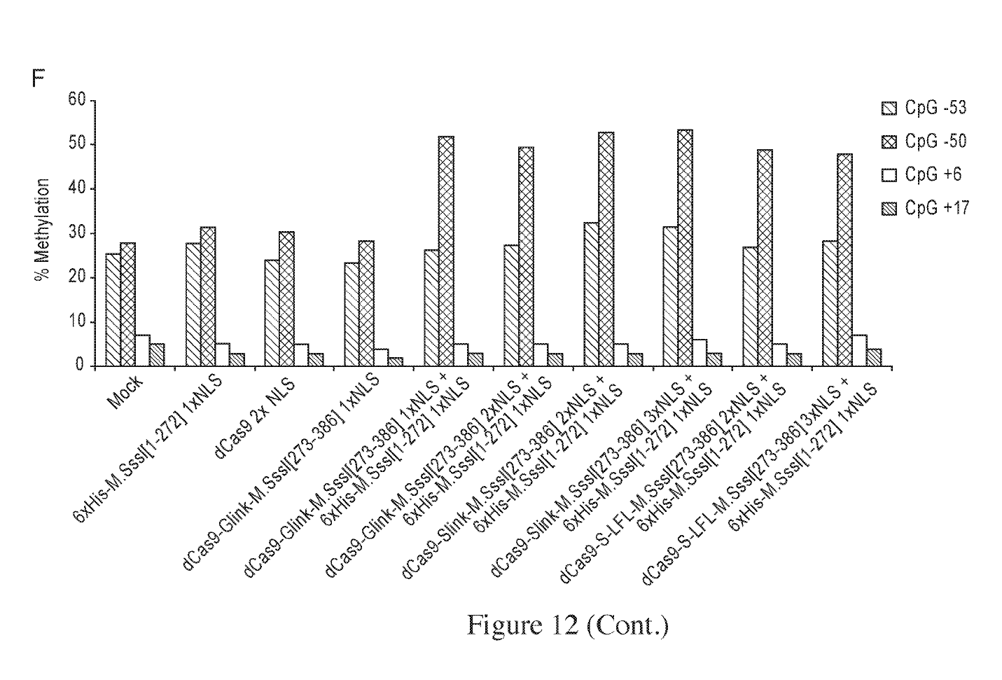

To test these variables in a systematic way several variants from both M.SssI fragments were created. For the first experiment, variants that had a nuclear localization from the nucleoplasmin protein (nucleoplasmin NLS) followed by a Flag tag (DYKDDDDK) fused to the N-terminus of dSPCas9 were created. Additionally, improvement of nuclear localization was assayed by fusing additional SV40 nuclear localization signals (SV40 NLS) either directly following the dSPCas9 sequence in the linker region or following the M.SssI [273-386] fragment. Three linker variants were also tested which are predicted to be unstructured allowing for a greater range of orientations. One is the previously used (GGGGS)3 linker. The other two linkers are used with versions including the SV40 nuclear localization which acts as part of the linker: one shorter (Slink) and one longer linker (S-LFL). The Slink is fused to the SV40 and has a single repeat of the flexible GGGGS sequence. The S-LFL is also fused to the SV40 NLS signal and contains smaller polar and non-polar residues (Ser, Thr, and Gly) while also containing larger polar and negatively charged residues to increase the hydrophilicity of the linker to allow for it move freely in aqueous solutions. These variants were paired with a single version of the free M.SssI[1-272] fragment containing a single SV40 NLS signal and 6× His tag fused the N-terminus (FIG. 12A), We attempted to target the dCas9-M.SssI[273-386] variants to a single site in the fetal hemoglobin promoter region (HBG) using the HBG F2 sgRNA. Note that there are actually two copies of the HBG (HBG1 and HBG2) which are nearly identical to each other. Our F2 sgRNA should be able to target both HBG genes and all assays were designed to try and sequence all 4 HBG alleles. There are two downstream CpG sites that are located 8 and 11 bp's away from the F2 sgRNA PAM site (FIG. 12B). A single CMV promoter drives expression of both the dCas9-M.SssI[273-386] as well as the free M.SssI[1-272] fragment. A separate U6 promoter expresses the HBG1 F2 sgRNA on the same plasmid (FIG. 2C).

To evaluate variants plasmids are transfected into HEK293T mammalian cells using the optifect reagent (Invitrogen) foin 6-well tissue culture plates. After 48 hours only cells expressing the GFP marker gene (and thus the M.SssI fragments) are collected and analyzed by bisulfite conversion followed by pyrosequencing using Pyromark Q24 advanced (Qiagen) (FIG. 12C). Primers were designed to sequence both the top and bottom strands at the −53 and −50 target CpG sites. Additionally a primer to sequence the top strand at two sites downstream (+6 and +17 sites) was also designed to evaluate off-target methylation (FIG. 12D). In addition to the constructs expressing both M.SssI fragments we evaluated four negative controls of Mock transfected cells (Optifect reagent but no plasmid), cells transfected with the M.SssI[1-272] only expressing plasmid and cells transfected with plasmids expressing the dCas9-M.SssI[273-386] or a dCas9 only without the M.SssI fragment attached (See schematics in FIG. 12E for various expected results of three negative controls and expression of both fragments). Data from the top and bottom strand were averaged at the −50 and −53 sites while data from the +6 and +17 sites are for only the top strand.

Results

M.SssI[1-272], dCas9 and dCas9-M.SssI[273-386] controls do not show any significant increase in methylation at the target sites compared to the Mock control and in the case where Cas9 proteins are localized at the site there is actually a slight decrease in methylation at the closer −53 (FIG. 1F). This decrease is presumably due to dCas9 binding blocking the site and preventing the natural methylation and was observed in multiple experiments. All variants co-expressing both the dCas9-M.SssI variants and the M.SssI[1-272 showed increased methylation at the −50 site on both the top and bottom strand, however no significant increases are seen at the −53 site—probably due to it being too close to the dCas9 binding site. Minor differences are seen for variants with the shorter Glink and S-link linkers. Variants with the longer S-LFL linker did not seem to be quite as active, however these variants also appear to be expressed in lower amounts when analyzed by western blots (data not shown). Western blots also show that there are slight increases in the amount of dCas9-M.SssI[273-386] in the nucleus when additional NLS signals are added to the dCas9-M.SssI constructs, however it does not appear to significantly increase methylation activity at the tested HBG 1 site.

Evaluation of Different Codon Optimization Strategies on dCas9-M.Sssl[273-386] and M.Sssl[1-272] Methylation Activities

Different codon optimizations of the M.SssI fragments and dSPCas9 were tested. The first version of the M.SssI fragments were designed to change any low frequency codons (<10-15% usage in the genome depending on residue) to higher frequency ones, and eliminate potential splice sites and termination signals in the sequence to ensure robust expression. Additionally any undesired restrictions sites for cloning purposes were removed. The dSPCas9 v1 was obtained from Jerry Peletier and was optimized by converting all codons in the sequence the highest frequency codon in humans for a given amino acid. The second versions (v2) for all M.SssI fragments and the dSPCas9 were designed to match the general frequency of codons for all residues between the human codons and the original species codon usage (i.e. match low frequency codon in S. pyogenes to low frequency in humans). Undesired restriction sites, possible splice sites and termination signals were also eliminated. This may allow for a more natural translation speed and improved folding and activity of proteins even if it reduces the overall amounts of protein produced in the cell.

We tried to co-express several versions of the dSPCas9-M.SssI[273-386] and M.SssI[1-272] by expressing them on separate plasmids. This allows for the testing of the M.SssI[1-272] and dCas9-M.SssI[273-386] variants in a combinatorial fashion. Expression on separate plasmids also allow for both fragments to be expressed off the strong pCMV promoter without the use of an IRES signal which could increase the expression of the M.SssI[1-272] proteins. The M.SssI[1-272] v2 variants differ only by the addition of a cmyc NLS sequence appended to the C-terminus of the fragments. The v1 versions differ in the N-terminal tag as we found that the initial 6× His tag was not detectable by western blot at its current site. The human influenza hemogglutinin (HA) tag (YPYDVPDYA) was added in place of the 6× His tag and allows for detection.

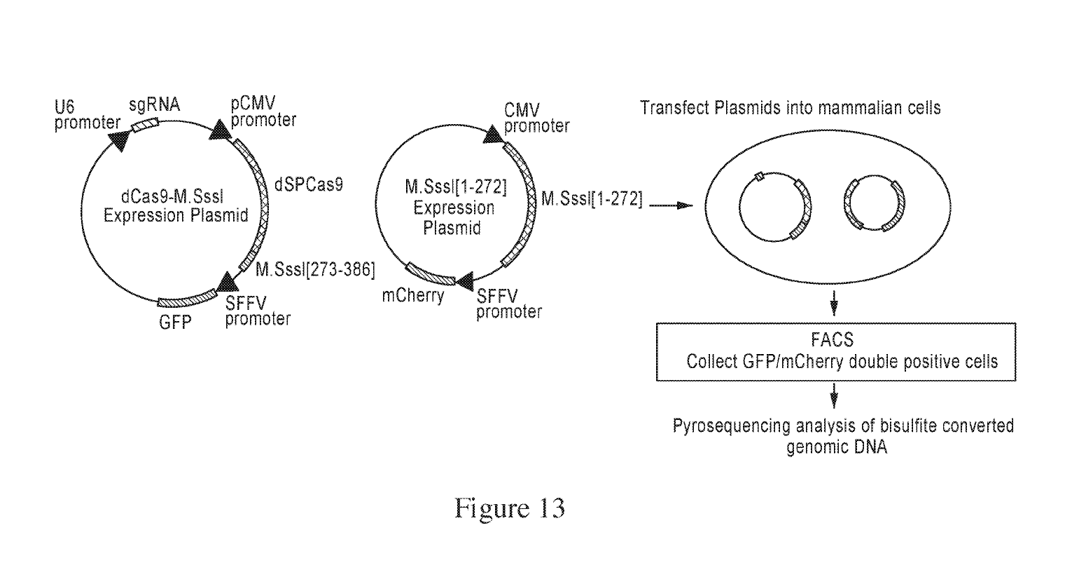

To evaluate methylation activity plasmids can be cotransfected into mammalian cell lines and sorted after 48 hours before analysis (see FIG. 13A). To ensure all cells that are analyzed express both M.SssI fragments, we cloned in separate fluorescent markers into the two plasmids: dSPCas9-M.SssI plasmids express eGFP and M.SssI.[1-272] plasmids express mCherry. Cotransfected cells can then be sorted for double positive cells containing both plasmids or sorted for single positive cells for samples where only one plasmid is transfected. After sorting, cells are collected and genomic DNA is converted using the Epitect Fast Bisulfite Conversion Kit. DNA can then be analyzed by pyrosequencing assays using sequencing primers shown in FIG. 12E.

Results

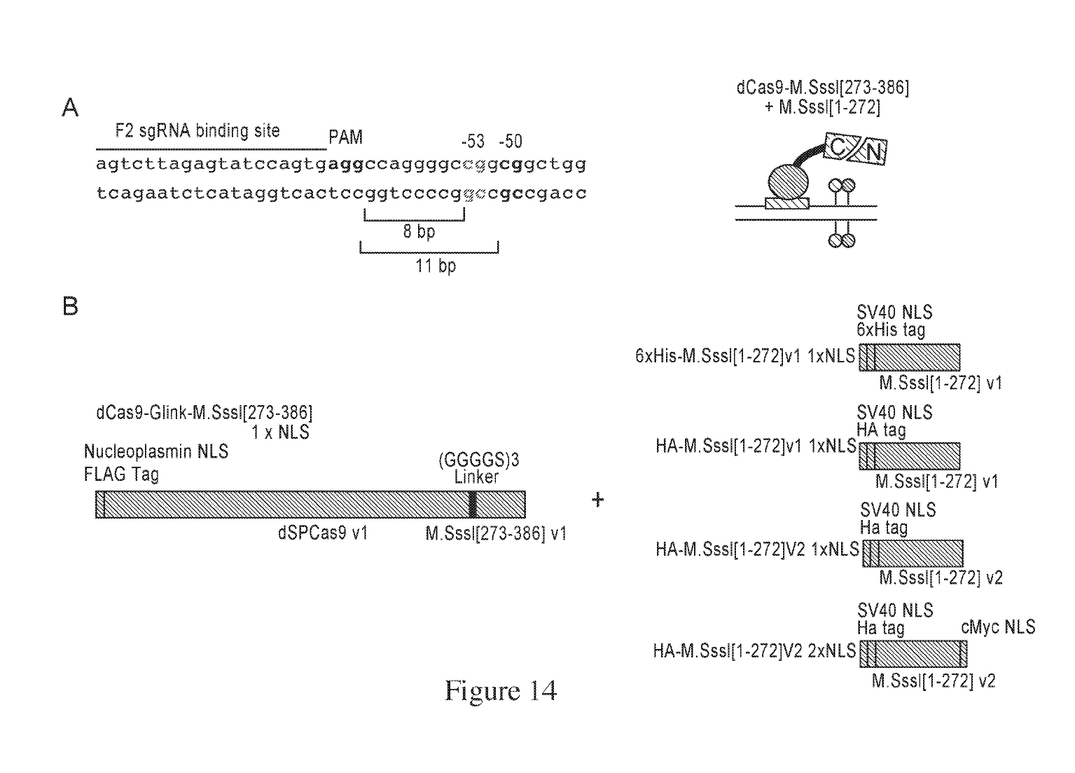

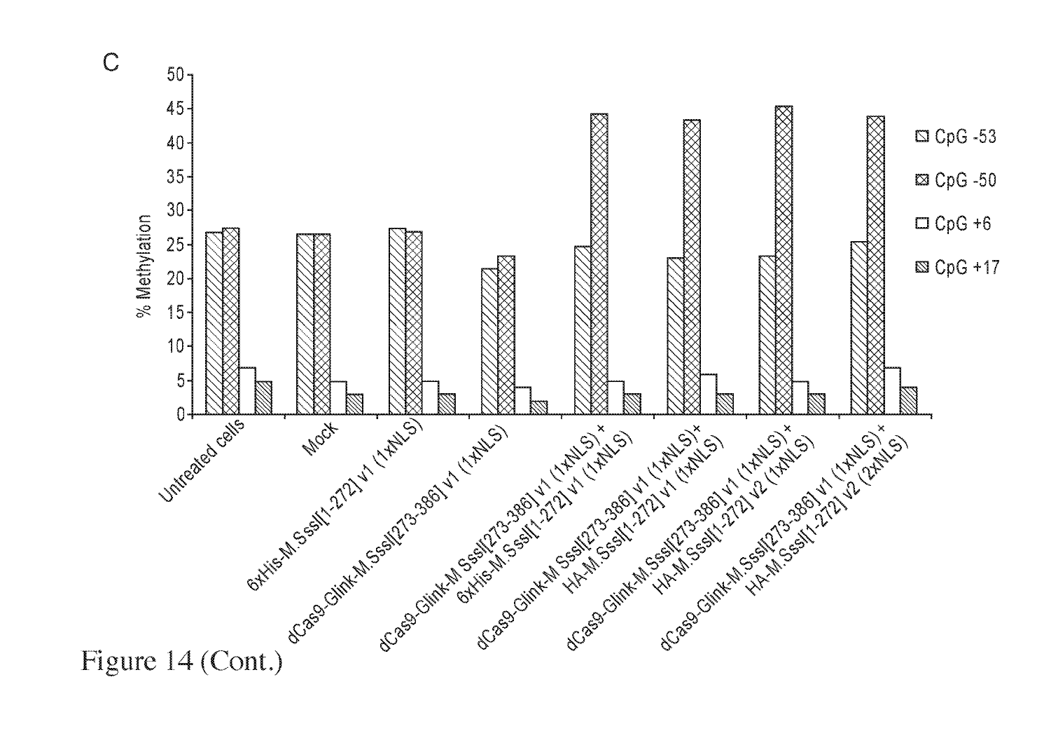

First we compared the methylation activity at the HBG1 promoter −53 and −50 sites (FIG. 14A) by cotransfection of our codon optimized version 1 dCas9-Glink-M.SssI[273-386] 1×NLS with various M.SssI[1-272] versions. Combinations tested in a single experiment are shown (FIG. 14B) along with untreated controls (cultured in same media conditions but without the optifect transfection reagent or plasmid), mock cells (optifect but no plasmid), and single plasmid variants of both the M.SssI[1-272] and dCas9-M.SssI[273-386]. All cotransfected samples showed increased methylation at the HBG1-50 site while levels at the −53 and two downstream off-target sites (+6 and +17) remain at similar level or decrease slightly (FIG. 14C). The decrease in methylation at the −53 site is probably due to blocking of the site by the dCas9 binding.

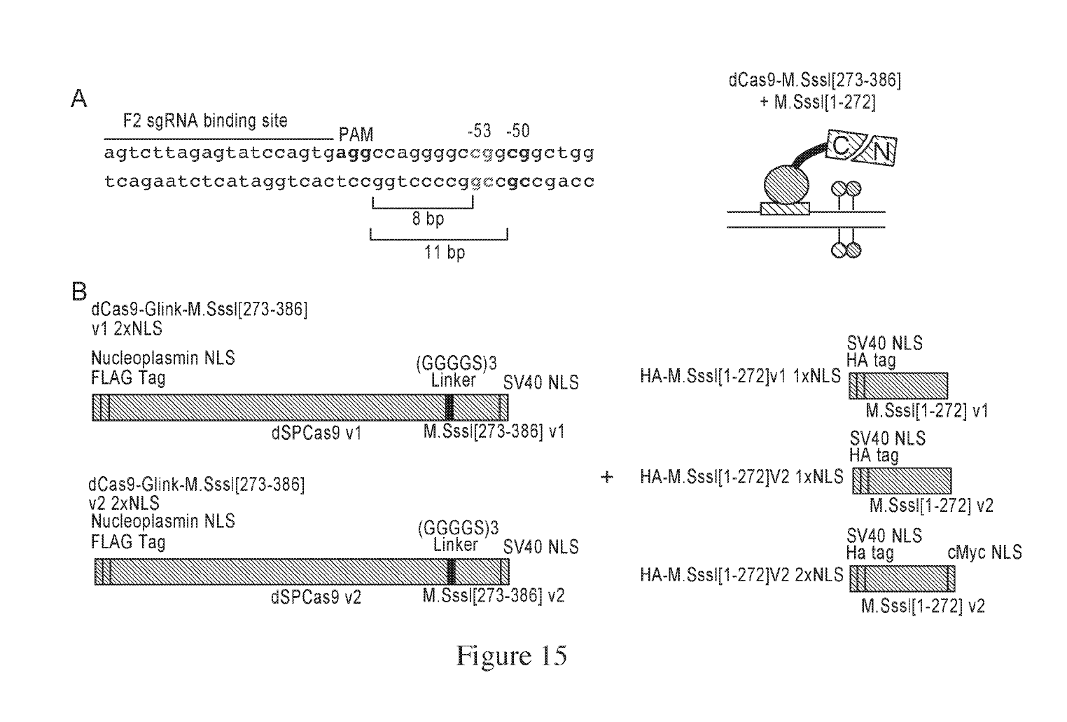

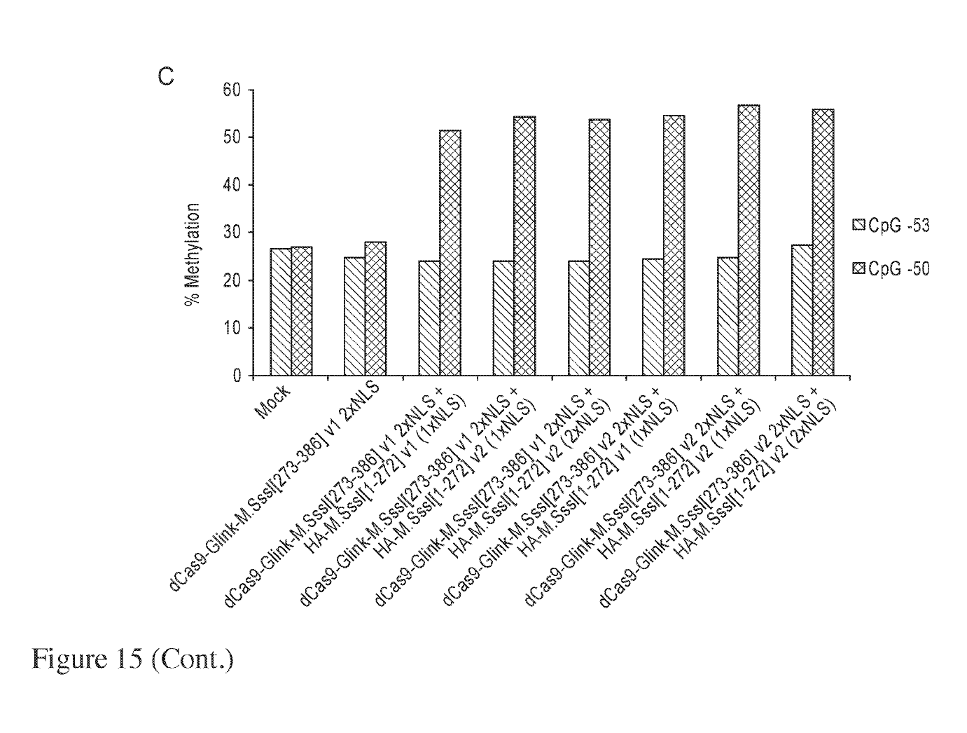

Second we performed similar experiments where we tested both the v 1 and v2 dCas9-Glink-M.SssI[273-386] 2×NLS variants with various M.SssI[1-272] constructs (FIG. 15). Again, the data indicate slightly higher methylation activity with our v2 optimized versions hut results are not significantly higher. However, there is a tendency for higher transfection efficiency and higher expression of GFP in cells from the v2 optimized constructs. Without being bound to any particular theory or hypothesis, this may be due to less toxicity of our variants. Assays are currently being developed to test this this hypothesis.

Fusion of the M.Sssl[273-386] to the N-Terminus of dSPCas9 and Evaluation of Methylation Activity at the HBG Promoters

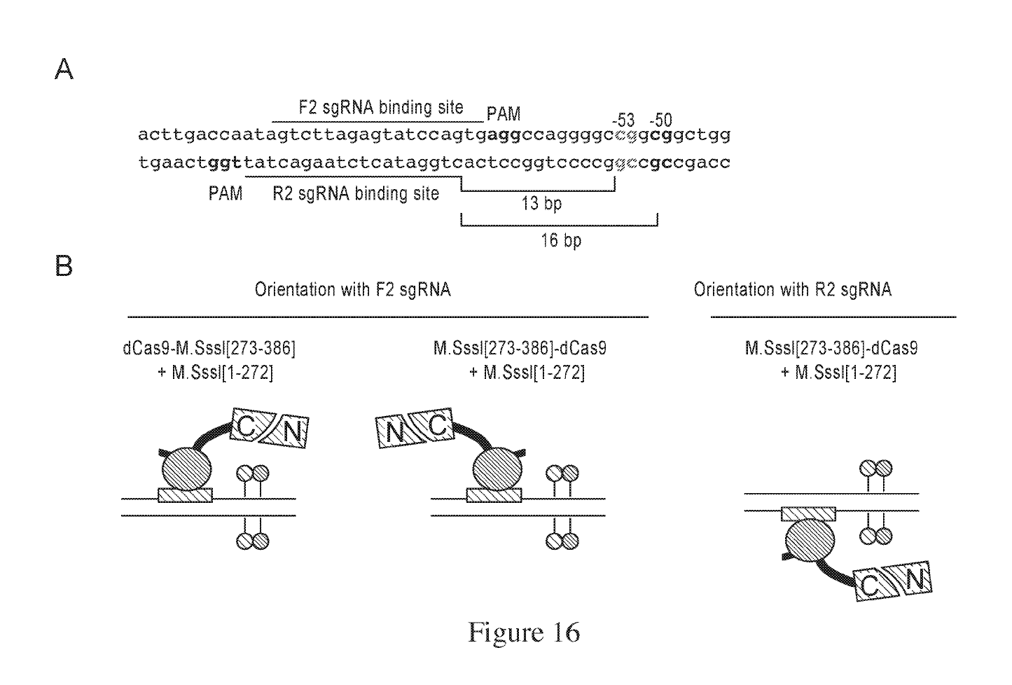

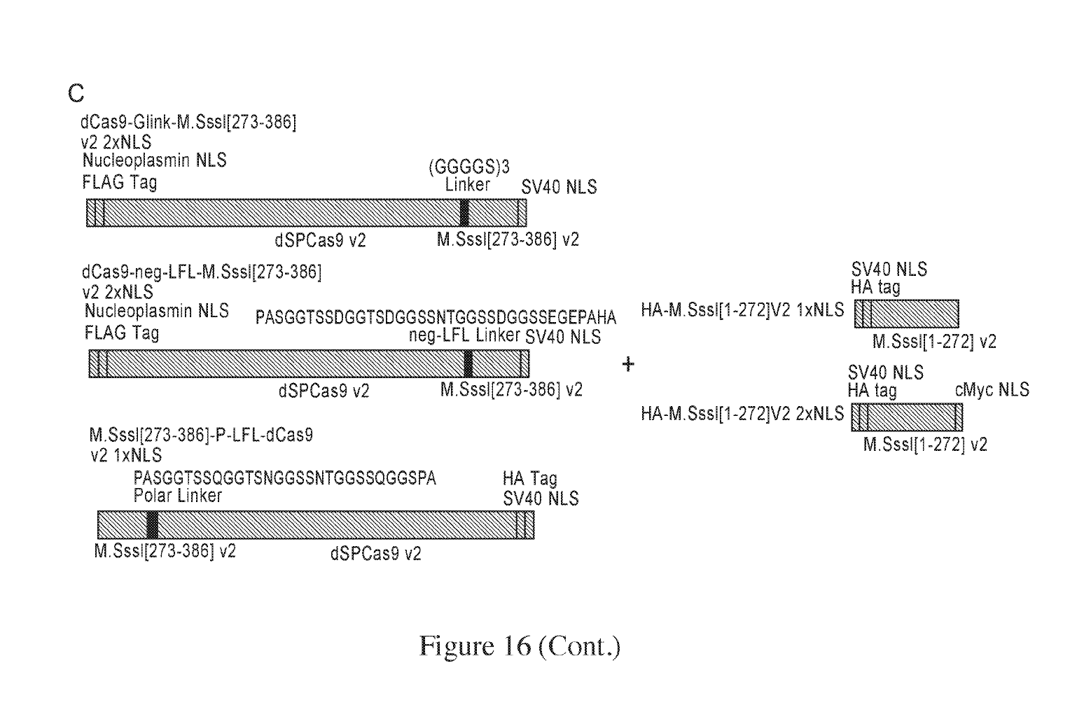

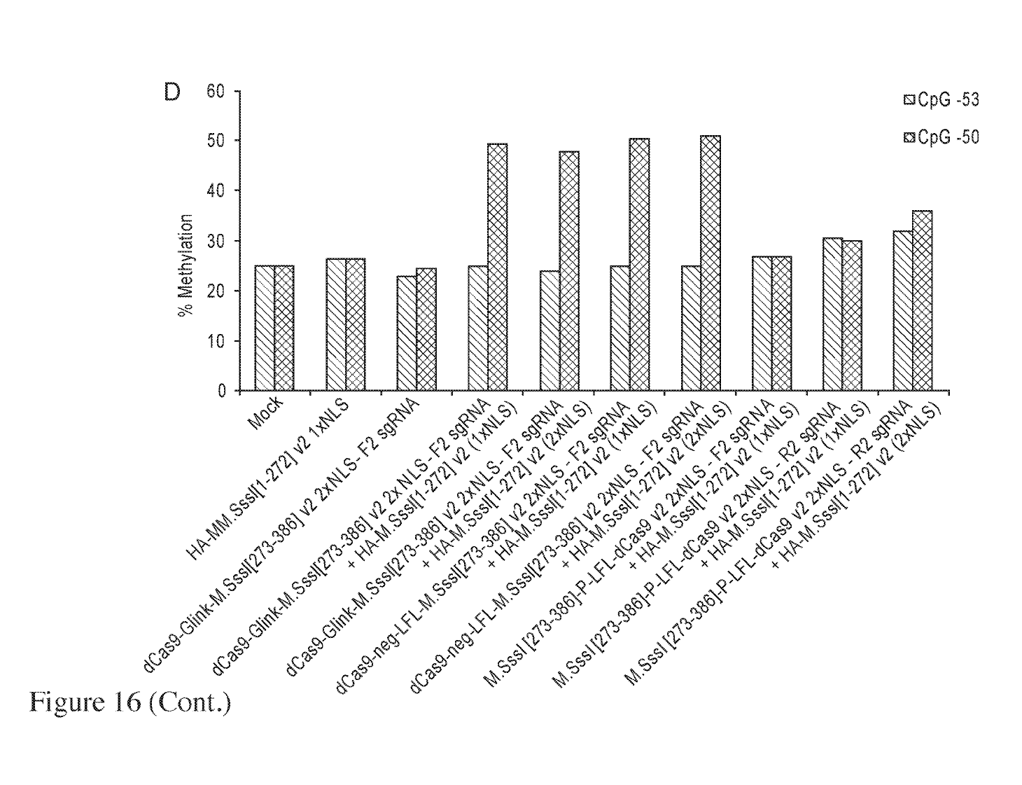

In many cases PAM sites might not be found a convenient length away from a target site or promoters may have a limited number of PAM sites. It would be useful to have the option of targeting sites on either side of the dCas9 binding site to expand the number of CpG sites that can be methylate without having to modify the dCas9 (or PAM binding site). Therefore we attempted to attach the M.SssI[273-386] fragment to the N-terminus of the dSPCas9 protein. This results in a very different spatial orientation in relation to dCas9 with the M.SssI[273-386] fragment localized to the DNA on the opposite side of the PAM binding site. This required a new design of the sgRNA to target the new construct to the same HBG −50 target site as previous constructs (See FIGS. 16A and B). A long flexible linker to fuse the C-terminus of M.SssI[273-386] to the N-terminus of the dSPCas9 protein was designed. This linker is similar to the previous S-LFL linker however it is not fused to a SV40 NLS and any charged residues of the neg-LFL linker and replaced them with larger polar residues. It is possible that a charged linker could have electrostatic interactions with the charged DNA backbone or charged residues in the histone proteins. Additionally, any N-terminal tags and NLS sequences were removed so that the constructs only have a C-terminal HA tag and SV40 NLS sequence fused to the dSPCas9 protein. Also tested was the previous dCas9-Glink-M.SssI[273-386] v2 2×NLS variant along with a new linker variant with an optimized codon long flexible linker with negatively charged residues (dCas9-neg-LFL-M.SssI[273-386] v2 2×NLS). Linkers and construct schemes are shown in FIG. 16C.

Results

Contracts for the dCas9-M.SssI[273-386] fusions showed similar methylation levels for both the Glink and neg-LFL linkers. While the new M.SssI[273-386]-P-LFL-dCas9 v2 1×NLS constructs did show an increase in methylation at both the −50 and −53 sites, it is significantly less than the dCas9-M.SssI[273-386] fusions (see FIG. 15D). Without being bound to any particular theory or hypothesis, it is possible that linker length, composition or the gap length between the dCas9 and target sites are suboptimal.

Methylation Activity at the SALL2 P2 Promoter Region with Bifurcated M.Sssl Fragments

As detailed above, the data indicate methylation at a specific site by targeting various M.SssI constructs to the HBG1 promoter. However, only a relative increase is observed of approximately 25-30% melthylation at the given site. Without being bound to any theory or hypothesis, it is possible that since there are four similar (but not identical) HBG promoters per genome there may be differences in accessibility due to higher order chromatin structure at different promoter sites limiting the ability to achieve higher methylation efficiency. Additionally the HBG promoters are CpG poor—having only 7 CpG sites in the ˜300 bp upstream of the translation start site. Because there are limited PAM sites available near the CpG sites, we were only able to try a small range of distances from the target methylation site. We therefore designed new sgRNA guide strands to target a promoter that had a higher density of CpG methylation sites.

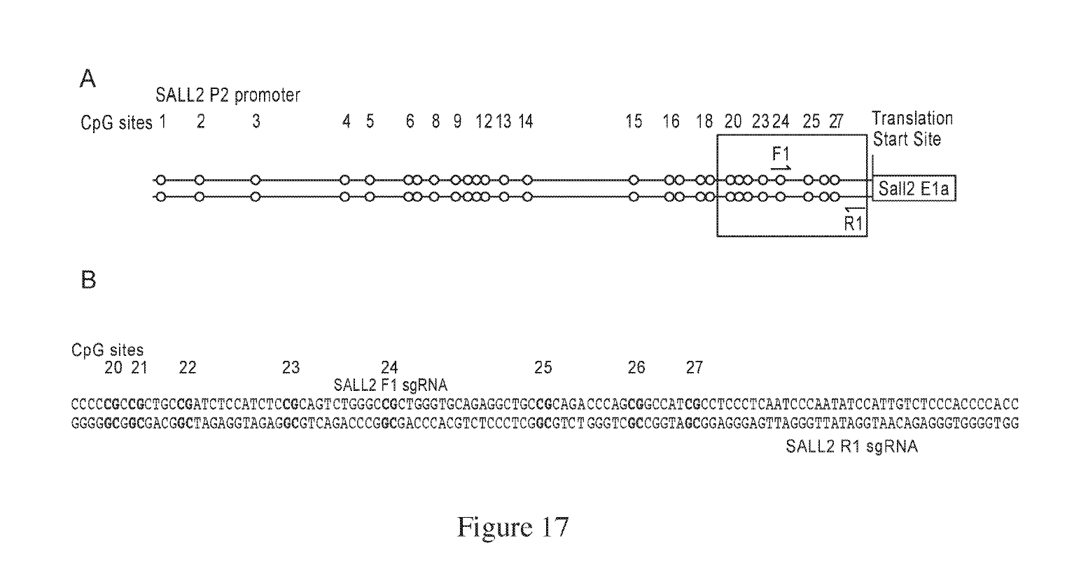

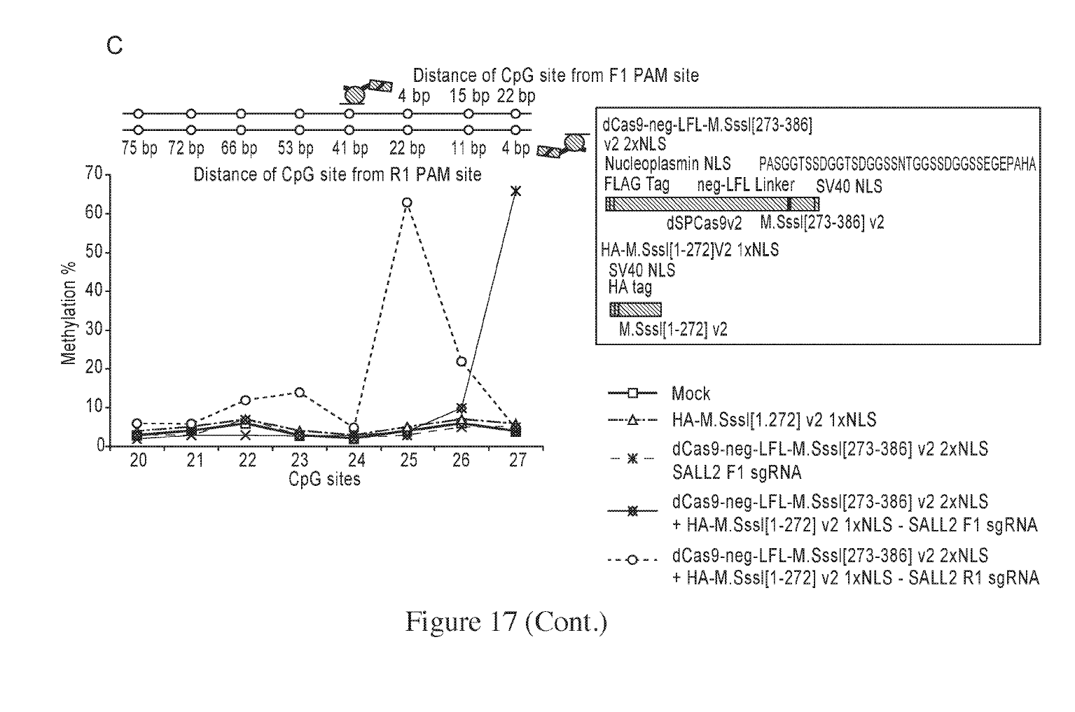

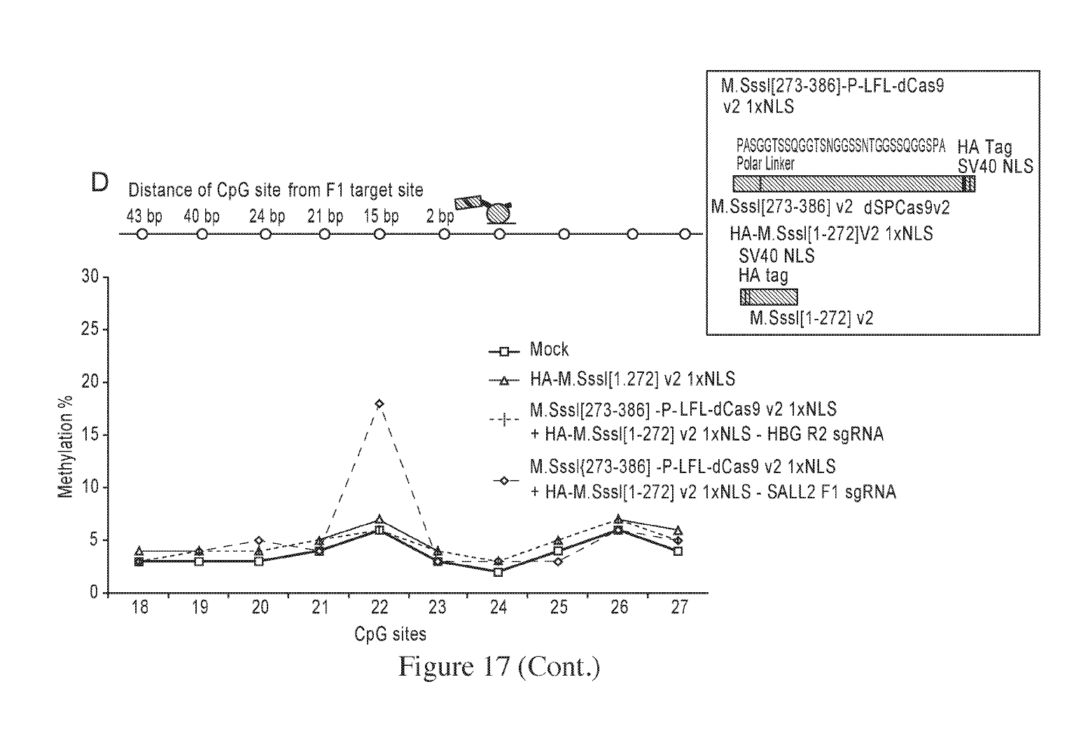

The SALL2 P2 promoter expresses the E1a isoform of SALL2 (aka p150) which is a putative tumor suppressor and has been found to be methylated in certain ovarian cancer cells. The promoter has a total of 27 CpG sites in the 550 bps upstream of the E1a isoform translation start site and a known CpG island between CpG 4 and 27 (FIG. 17A). We designed 2 guide strands—SALL2 F1 and SALL2 R1 to target the methylation sites closest to the translation start site (FIG. 17B). These sites are close in proximity to multiple CpG sites and will allow us to evaluate a variety of gap lengths in the context of genomic DNA. Gap lengths (listed as CpG distances from the end of the sgRNA or PAM sites) are shown with the results graphs (FIGS. 17C and D). Both M.SssI[273-386]-dCas9 and dCas9-M.SssI[273-386] constructs were tested as they are capable of methylating different sites using the same sgRNA target site (F1). These were cotransfected with plasmids for expression of a single M.SssI[1-272] variant.

Results

SALL2 P2 is normally hypomethylated in HEK293T cells with initial evaluation of the cell line showing methylation over the region consistently under 10%. Mock controls show similarly low levels of methylation with the majority of sites between 2-6% methylated (FIG. 17C and D). Other negative controls including a single expression plasmid transfection of HA-M.SssI[1-272] v2 1×NLS or dCas9-neg-LFL-M.SssI[273-386] v2 2×NLS targeted to the SALL2 F1 site show nearly identical levels of methylation (FIG. 17C). Only samples coexpressing both M.SssI fragments show significantly higher levels of methylation. In the case of the dCas9-neg-LFL-M.SssI[273-386] fusion samples (shown in FIG. 17C) significantly higher levels of methylation (>60%) are found at a sites with gap lengths 22 by away from both the SALL2 F1 and SALL2 RI target sites. Interestingly both samples also show intermediate levels of methylation at the CpG 26 site (15 bp from the F1 PAM site and 11 bp from the R1 PAM site) with slightly higher levels (˜20% methylation) with the SALL2 F1 sgRNA. Unfortunately there are not any sites analyzed past the CpG 27 site for the SALL2 F1 sgRNA sample, but we were able to analyze sites further away from the SALL2 R1 sgRNA. Methylation peaks at the CpG 25 site (22 bp gap length) but drops again to background levels at CpG 24 (41 bp). Methylation increases slightly at the CpG 23 and 22 sites again (53 and 66 bp away).

The single sample with M.SssI[273-3861-P-LFL-dCas9 targeted to the SALL2 P2 promoter did show an slight increase in methylation (12% increase) at a site 15 bp away (CpG 22), similar to levels seen at the HBG experiment in FIG. 16. The control expressing both M.SssI fragments but with a sgRNA targeting the dCas9 fusion to the HBG promoter F2 site shows no methylation over background at the same SALL2 CpG22 site.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A system comprising:
a bifurcated enzyme comprising a first fragment and a second fragment wherein:
a. the first fragment, the second fragment or both further comprise a DNA binding domain that bind elements flanking a target region; and
b. the system has been optimized for expression in a mammalian cell.

2. The system of claim 1, wherein the DNA binding domain binds elements upstream, or downstream of the target region.

3. The system of claim 1, wherein the first fragment comprises the N-terminal portion of the enzyme and the second fragment comprises the C-terminal portion of the enzyme.

4. The system of claim 3, wherein the second fragment comprises the DNA binding domain.

5. The system of claim 1, further comprising a linker between the enzyme fragment and the DNA binding domain.

6. The system of claim 1, further comprising a nuclear localization signal.

7. The system of claim 1, wherein the enzyme is a DNA methyltransferase.

8. The system of claim 7, wherein the first fragment comprises a portion of the catalytic domain of the DNA methyltransferase.

9. The system of claim 7, wherein the DNA methyltransferase is M.SssI.

10. The system of claim 9, wherein the first fragment comprises amino acids 1-272 of the M.SssI.

11. The system of claim 10, wherein the second fragment comprises amino acids 273-386 of the M.SssI.

12. The system of claim 1, wherein the enzyme is a DNA demethylase.

13. The system of claim 1, wherein the target region comprises a CpG methylation site.

14. The system of claim 1, wherein the target region is within a promoter region.

15. The system of claim 1, wherein the DNA binding domain a zinc finger, a TAL effector DNA-binding domain or a RNA-guided endonuclease and a guide RNA.

16. The system of claim 15, wherein the guide RNA is complementary to the region flanking the target region.

17. The system of claim 15, wherein the RNA-guided endonuclease is a CAS9 protein.

18. The system of claim 17, wherein the CAS9 protein has inactivated nuclease activity.

19. A plurality of systems according to claim 1, wherein the DNA binding domain of each system binds a different site in genomic DNA.

20. A fusion protein comprising an RNA guided nuclease and a first portion of a bifurcated methyltransferase, wherein the fusion protein is expressed in a mammalian cell.

21. The fusion protein of claim 20, wherein the RNA guided nuclease is a CAS9 protein having inactivated nuclease activity.

22. An expression cassette comprising a nucleic acid encoding a bifurcated methyltransferase, a DNA binding domain and a mammalian promoter.

23. A mammalian cell stably expressing the expression cassette according to claim 22.

24. A reporter plasmid comprising a backbone free of any methylation sites having a target promoter sequence inserted upstream of a nucleic acid encoding a first fluorescent protein and a control promoter sequences inserted upstream of a nucleic acid encoding a second fluorescent protein.

25. The plasmid of claim 24, wherein the first fluorescent protein is mCherry and the second fluorescent protein is mTAGBFP2.

26. The plasmid of claim 24, wherein the target promoter is methylation sensitive.

27. The plasmid of claim 24, wherein the control promoter is not methylation sensitive.

28. The plasmid of claim 24, wherein the control promoter is CpG free EF1.

29. The plasmid of claim 24, wherein the target promoter and the control promoter is methylation sensitive

30. A cell comprising the plasmid of claim 24.

31. The cell of claim 30, further comprising an expression plasmid comprising a DNA demethylase or DNA methyltransferase fused to a DNA binding domain.

32. The cell of claim 23, transfected with the reporter plasmid of claim 16.

33. A method of identifying a functionally repressive CpG site in a target promoter comprising:
contacting the cell of claim 32 with a plurality of guide RNAs;
measuring the fluorescent intensity of the first and second fluorescent protein.

34. A method of epigenetic reprogramming a mammalian cell comprising contacting the cell with the system of claim 1.

35. A method of epigenetic therapy comprising administering to a mammalian subject in need thereof a composition comprising the system of claim 1.

36. The method of claim 35, wherein said subject has cancer, a hematologic disorder, a neurodegenerative disorder, heart disease, diabetes, or mental illness.

37. The method of claim 35, wherein the hematologic disorder is sickle cell or thalessemia.

38. The method of claim 35, wherein the cancer is lymphoma.
